(19) United States
(12) Patent Application Publication
(10) Pub. No.: US 2025/0072916 A1
(43) Pub. Date: Mar. 6, 2025

(54) SYSTEM AND METHOD FOR A BUNIONECTOMY VIA BONE CUTTING AND FIXATION OF A METATARSAL IN THE FOOT

(71) Applicant: Tzvi BAR-DAVID, Bronx, NY (US)

(72) Inventor: Tzvi BAR-DAVID, Bronx, NY (US)

(21) Appl. No.: 18/825,323

(22) Filed: Sep. 5, 2024

Related U.S. Application Data
(60) Provisional application No. 63/536,759, filed on Sep. 6, 2023.
(60) Provisional application No. 63/663,964, filed on Jun. 25, 2024.
(60) Provisional application No. 63/679,046, filed on Aug. 2, 2024.

(51) Int. Cl. A61B 17/17; A61B 17/15; A61B 17/56; A61B 17/80

(52) U.S. Cl. CPC A61B 17/1775; A61B 17/151; A61B 17/8061; A61B 17/808; A61B 2017/564

(57) ABSTRACT

A system and method for a ROSE-Reverdin osteotomy obviates severe deformities having a high intermetatarsal angle and which require closure via osteotomy (bone cut) of the first and/or fifth metatarsal in the midshaft or proximal aspect of the bone. The ROSE osteotomy is utilized to achieve this correction and is performed without need for fusion of the first metatarsal cuneiform joint. The ROSE osteotomy achieves correction of the intermetatarsal angle via rotation of the first and/or fifth metatarsal about a proximal based axis point. The Reverdin osteotomy is required to correct a deviated articular cartilage deformity at the head of the first metatarsal and/or additional slight correction of the IM angle. The Reverdin osteotomy achieves correction by wedging of the cut to realign the cartilage and translation of the head of the first metatarsal for further IM correction. A unique jig is used to achieve this cut and fixation.

### CROSS REFERENCE TO RELATED APPLICATIONS

The present application claims the benefit under 35 U.S.C. § 119 of U.S. Provisional Patent Application No. 63/536,759, filed on Sep. 6, 2023, and U.S. Provisional Patent Application No. 63/663,964, of the same title and filed on Jun. 25, 2024, and U.S. Provisional Patent Application No. 63/679,046, also of the same title and filed on Aug. 2, 2024, the contents of each which are incorporated herein by reference.

### BACKGROUND OF THE DISCLOSURE

### 1. Field of the Disclosure

The present disclosure relates to a way to perform bunionectomy procedures which require closure or reduction of the space between the first and second metatarsals, and the fourth and fifth metatarsals called the intermetatarsal space. The intermetatarsal space is measured by the intermetatarsal (IM) angle between the first and second, or fourth and fifth, metatarsal bones. Advanced deformities have a high intermetatarsal angle and require closure via osteotomy (bone cut) of the first and/or fifth metatarsal in the midshaft or proximal aspect of the bone. More particularly, it relates to a system and method for a Rotational Osteotomy System for Enhanced (hereinafter “ROSE”) bunionectomy procedure/bunion correction, formerly known as a Mau osteotomy, which is utilized to achieve this correction and is done without the need for fusion of the first metatarsal cuneiform joint which bears its own set of complications. The ROSE bunionectomy procedure (e.g., ROSE osteotomy and/or ROSE bunion correction) achieves correction of the intermetatarsal angle via rotation of the first and/or fifth metatarsal about a proximal based axis point. The ROSE bunionectomy procedure is a proximal mid-shaft rotational osteotomy of the first metatarsal. Further, a second osteotomy, the Reverdin osteotomy, is required to correct a deviated articular cartilage deformity at the head of the first metatarsal and/or an additional slight correction of the IM angle. Thus, correction with this osteotomy is achieved by wedging of the cut to realign the cartilage and translation of the head of the first metatarsal for further IM correction. A unique jig is used to achieve this cut and fixation.

### 2. Description of the Related Art

Conventional techniques with respect to bunion correction or repair require the fusing of bones and/or joint fusion, such as Lapidus and/or Lapiplasty® (trademark owned by Treace Medical Concepts, Inc.) bunionectomy procedures for bunion correction, wherein the first tarsometatarsal joint is fused to correct deformity of the joint and to prevent further movement of the joint. However, existing Lapidus and/or Lapiplasty® bunionectomy techniques may be difficult to perform with the desired precision. Further, these conventional procedures require a longer recovery time, provide limited foot mobility, require the patient to remain off his/her feet for a longer period of time, cause a decreased range of motion due to elevation of the first metatarsal, and result in pain beneath the lesser metatarsal (i.e., Metatarsalgia) due to shortening of the first metatarsal.

Bunion correction or repair is a common surgery with over 100,000 surgeries performed annually in the U.S. Bunions are a progressive disorder typically beginning with a leaning of the big toe toward the second toe, which can gradually change the angle of the bones and produce a characteristic bump on the medial side of the metatarsal near the joint of the metatarsal with the proximal phalanx. Specifically, the bunion is the prominence made of bone and at times an inflamed bursa. Hallux abductus of the transverse plane is the condition in which the big toe deviates from the normal position toward the direction of the second toe.

There exist systems which require fusing of bones and/or joints, require a longer recovery time, provide limited foot mobility, require the patient to remain off his/her feet for a longer period of time, cause a decreased range of motion due to elevation of the first metatarsal, and result in pain beneath the lesser metatarsal (i.e., Metatarsalgia) due to shortening of the first metatarsal. Existing prior art can be clustered into two groups of solutions: Lapidus and/or Lapiplasty®. Additionally, attempting to perform the fusing of bones and/or joints with the system and method of the present disclosure would not produce consistent results, thus severely limiting the viability of the procedure.

There is a need for a system and method that does not require the fusing of bones and/or joints, has a quicker period of healing, causes less discomfort both while healing and when healed, and allows the patient to be back on his/her feet following foot surgery. Further, there is a need for a way to allow for consistent reliable reproduction of the osteotomy cut, rigid internal fixation to allow for early weightbearing, and adequate correction of the deformity to minimize recurrence, as well as, avoiding the complication of nonunion of the metatarsal cuneiform joint (i.e., the tarsometatarsal joint). The only way to do this with the prior art is by Lapidus and/or Lapiplasty® bunionectomy techniques, which may be difficult to perform with the desired precision, require fusing of bones and/or joints, require a longer recovery time, provide limited foot mobility, require the patient to remain off his/her feet for a longer period of time, cause a decreased range of motion due to elevation of the first metatarsal, and result in pain beneath the lesser metatarsal (i.e., Metatarsalgia) due to shortening of the first metatarsal. Thus, in order to reduce the complications of conventional bunionectomy techniques, a unique system and method for a bunionectomy procedure, unique osteotomy jigs, a unique IM correction bone clamp, a unique bone plate with screws, and a novel clamping screw have been designed to achieve these goals.

### SUMMARY OF THE DISCLOSURE

Example embodiments described herein have innovative features, no single one of which is indispensable or solely responsible for their desirable attributes. Without limiting the scope of the claims, some of the advantageous features will now be summarized. The term “osteotomy” and “bone cut” may be used interchangeably throughout the disclosure described herein.

An “osteotomy” is a surgical operation whereby “a bone is cut” to shorten or lengthen it or to change its alignment. It is sometimes performed to correct a hallux valgus, hallux abductus valgus, or to straighten a bone that has healed crookedly following a fracture. Further, it is also used to correct a coxa vara, genu valgum, and genu varum.

In a first aspect, an embodiment of the disclosure is directed to a unique surgical procedure for correcting advanced bunions without the need for joint fusion, setting it apart from other methods like Lapidus and/or Lapiplasty®. Joint fusion involves fusing bones together to correct the bunion. This can lead to longer recovery times and might limit the foot's movement. With the approach described herein, joint fusion is avoided, aiming to bring the patient back to his/her regular activities, reduce pain, and make his/her feet feel more comfortable.

Avoiding joint fusion means quicker healing and less discomfort. The patient can even put some weight on his/her foot immediately after the surgery, making the recovery easier. Unlike those methods that require joint fusion, this unique approach preserves joint function, reducing the risk of complications and promoting a quicker and more comfortable recovery.

In general, an embodiment of the disclosure is directed to a system and method for allowing for consistent reliable reproduction of the osteotomy cut, rigid internal fixation to allow for early weightbearing, and adequate correction of the bunion deformity to minimize recurrence, as well as, avoiding the complication of nonunion of the metatarsal cuneiform joint (i.e., the tarsometatarsal joint), wherein unique osteotomy jigs, an IM correction bone clamp, and a bone plate with screws, have been designed to achieve these goals. The embodiment of the disclosure is further directed to a system and method utilized to achieve this correction and is done without the need for fusion of the first metatarsal cuneiform joint which bears its own set of complications. Thus, the system and method achieve correction of the intermetatarsal angle via rotation of the first and/or fifth metatarsal about a proximal based axis point. Further, the system and method also address correction of the cartilage end joint to correct a deviated articular cartilage deformity at the head of the first metatarsal and/or additional slight correction of the IM angle. Correction with this osteotomy is achieved by wedging of the cut to realign the cartilage and translation of the head of the first metatarsal for further IM correction. A unique jig is used to achieve this cut and fixation.

The system and method are qualitatively different from the prior art in that they significantly build upon it by utilizing bespoke individual measures that are taken to address the surgical intractability of working with the fusion of bones and/or joints. The techniques disclosed herein also enable bunionectomy procedures that are qualitatively different from anything that was possible with the prior art in most cases.

The surgeon can reliably perform the bunionectomy surgical procedure with minimal difficulty. The patient can reliably receive a shorter healing period, less discomfort, quicker gain of mobility, and better long-term results with no need for the fusing of bones and/or joints. The major advantages include scale, reproducibility of medical procedure, ability to quantify/qualify results and/or future mobility, and enabling future methods, particularly supervised and/or unsupervised AI to assist the surgeon in the bunionectomy procedure disclosed herein.

An embodiment of the disclosure is directed to a system and method for utilizing a bone cut to achieve correction of deformities having a high intermetatarsal angle, though can be used for lesser intermetatarsal angles.

Another embodiment of the disclosure is directed to a system for utilizing a bone cut to achieve correction of deformities having an intermetatarsal angle, the system comprising: a first bone cut jig; at least one support that secures the first bone cut jig to a metatarsal; a first jig cut guide groove in the first bone cut jig, wherein a cutting tool traverses the first jig cut guide groove, thereby creating a bone cut through the metatarsal from medial to lateral, wherein the bone cut is oriented from dorsal distal to plantar proximal; a plurality of dorsal holes disposed in a dorsal surface of the first bone cut jig, thereby utilizing said support to hold the first bone cut jig to a dorsal surface of the metatarsal, wherein at least one of the plurality of dorsal holes is proximal and at least one of the plurality of dorsal holes is distal to the bone cut, dorsal exit point; a plurality of medial holes disposed in a medial surface of the first bone cut jig, thereby utilizing said support to hold the first bone cut jig to a medial surface of the metatarsal, wherein at least one of the plurality of medial holes is superior proximal and at least one of the plurality of medial holes is distal inferior to the first jig cut guide groove; a bone cut fixation plate placed over the dorsal surface of the metatarsal, wherein the bone cut fixation plate has a slight bend distally; and a plurality of fixation fasteners that secure the bone cut fixation plate to the dorsal surface of the metatarsal, wherein said support is utilized, thereby securing the bone cut fixation plate proximal and distal as the plurality of fixation fasteners are being inserted.

Yet another embodiment of the disclosure is directed to the system further comprising: a bone cut IM correction clamp placed on the dorsal surface of the metatarsal, thereby as the bone cut IM correction clamp is closed, the metatarsal is rotated laterally closing or reducing the intermetatarsal (IM) angle, thus correcting the deformities.

Still yet another embodiment of the disclosure is directed to the system further comprising: a second bone cut jig; a plurality of fixation holes disposed in a dorsal surface and a medial surface of the second bone cut jig to stabilize the second bone cut jig on a head of the metatarsal; a plurality of supports for utilizing with the plurality of fixation holes, thereby stabilizing the second bone cut jig to the head of the metatarsal; and at least one second jig cut guide groove.

Another embodiment of the disclosure is directed to a method for utilizing a bone cut to achieve correction of deformities having an intermetatarsal angle, the method comprising: fitting a first bone cut jig; inserting at least one support that secures the first bone cut jig to a metatarsal; fitting a first jig cut guide groove in the first bone cut jig, wherein a cutting tool traverses the first jig cut guide groove, thereby creating a bone cut through the metatarsal from medial to lateral, wherein the bone cut is oriented from dorsal distal to plantar proximal; utilizing a plurality of dorsal holes disposed in a dorsal surface of the first bone cut jig, thereby utilizing said support to hold the first bone cut jig to a dorsal surface of the metatarsal, wherein at least one of the plurality of dorsal holes is proximal and at least one of the plurality of dorsal holes is distal to the bone cut, dorsal exit point; utilizing a plurality of medial holes disposed in a medial surface of the first bone cut jig, thereby utilizing said support to hold the first bone cut jig to a medial surface of the metatarsal, wherein at least one of the plurality of medial holes is superior proximal and at least one of the plurality of medial holes is distal inferior to the first jig cut guide groove; fitting a bone cut fixation plate placed over the dorsal surface of the metatarsal, wherein the bone cut fixation plate has a slight bend distally; and utilizing a plurality of fixation fasteners that secure the bone cut fixation plate to the dorsal surface of the metatarsal, wherein said support is utilized, thereby securing the bone cut fixation plate proximal and distal as the plurality of fixation fasteners are being inserted.

Yet another embodiment of the disclosure is directed to the method further comprising: utilizing a bone cut IM correction clamp placed on the dorsal surface of the metatarsal, thereby as the bone cut IM correction clamp is closed, the metatarsal is rotated laterally closing or reducing the intermetatarsal (IM) angle, thus correcting the deformities.

Still yet another embodiment of the disclosure is directed to the method further comprising: fitting a second bone cut jig; utilizing a plurality of fixation holes disposed in a dorsal surface and a medial surface of the second bone cut jig to stabilize the second bone cut jig on a head of the metatarsal; using a plurality of supports for utilizing with the plurality of fixation holes, thereby stabilizing the second bone cut jig to the head of the metatarsal; and utilizing at least one second jig cut guide groove.

A further embodiment of the disclosure is directed to a system for utilizing a bone cut to achieve correction of deformities of a head of a metatarsal, the system comprising: a second bone cut jig; a plurality of fixation holes disposed in a dorsal surface and a medial surface of the second bone cut jig to stabilize the second bone cut jig on the head of the metatarsal; a plurality of supports for utilizing with the plurality of fixation holes, thereby stabilizing the second bone cut jig to the head of the metatarsal; and at least one second jig cut guide groove.

Yet a further embodiment of the disclosure is directed to the system, wherein the at least one second jig cut guide groove is selected from the group consisting of: a wedge shaped and dorsal, a straight linear shaped and medial, and a fully through a dorsal surface.

Still yet another embodiment of the disclosure is directed to a method for utilizing a bone cut to achieve correction of deformities of a head of a metatarsal, the method comprising: fitting a second bone cut jig; utilizing a plurality of fixation holes disposed in a dorsal surface and a medial surface of the second bone cut jig to stabilize the second bone cut jig on the head of the metatarsal; using a plurality of supports for utilizing with the plurality of fixation holes, thereby stabilizing the second bone cut jig to the head of the metatarsal; and utilizing at least one second jig cut guide groove.

Still yet a further embodiment of the disclosure is directed to the method, wherein the at least one second jig cut guide groove is selected from the group consisting of: a wedge shaped and dorsal, a straight linear shaped and medial, and a fully through a dorsal surface.

Another embodiment of the disclosure is directed to a system for utilizing a bone cut to achieve correction of deformities having an intermetatarsal angle, the system comprising: a first bone cut jig that fits the dimension and contour of a dorsal and medial surface of a metatarsal of a bone; at least one support disposed such that it secures the first bone cut jig to the bone; a jig cut guide groove in the first bone cut jig for fitting and placement of a cutting tool, wherein the cutting tool traverses the jig cut guide groove, thereby creating a bone cut through the metatarsal from medial to lateral, wherein the bone cut is oriented from dorsal distal to plantar proximal; a plurality of dorsal holes disposed in a dorsal surface of the first bone cut jig for securing the first bone cut jig to the bone, thereby utilizing at least one support, oriented dorsal to plantar for temporarily holding the first bone cut jig to the dorsal surface of the metatarsal, wherein at least one of the plurality of dorsal holes is proximal and at least one of the plurality of dorsal holes is distal to the bone cut, dorsal exit point; a plurality of medial surface holes disposed in a medial surface of the first bone cut jig for securing the first bone cut jig to the bone, thereby utilizing at least one support for temporarily holding the first bone cut jig to the medial surface of the metatarsal, wherein at least one of the plurality of medial surface holes is superior proximal and at least one of the plurality is distal inferior to the jig cut guide groove; a bone cut IM correction clamp placed on the dorsal surface of the metatarsal after the bone cut is completed and a rotation axis support is placed, thereby as the bone cut IM correction clamp is closed down the metatarsal is rotated laterally closing down or reducing an intermetatarsal (IM) angle, thus substantially correcting the deformities; a bone cut fixation plate placed over the dorsal surface of the metatarsal to fix the bone cut rigidly, wherein the bone cut fixation plate has a slight bend distally to maintain a position dorsal on a distal segment of the bone as the intermetatarsal (IM) angle is reduced; and a plurality of fixation fasteners for securing the bone cut fixation plate permanently via dorsal holes disposed in a dorsal surface of the bone cut fixation plate such that it secures the bone cut fixation plate to the dorsal surface of the metatarsal, wherein at least one support is utilized, thereby temporarily securing the bone cut fixation plate proximal and distal as the plurality of fixation fasteners are being inserted.

Yet another embodiment of the disclosure is directed to the system further comprising: a second bone cut jig that fits dorsal and medial over a head of a metatarsal, wherein the second bone cut jig faces dorsal and medial; a plurality of fixation holes disposed in a dorsal surface and a medial surface of the second bone cut jig for temporarily stabilizing the second bone cut jig on the head of the metatarsal; a plurality of supports for utilizing with the plurality of fixation holes, thereby temporarily stabilizing the second bone cut jig to the head of the metatarsal; and at least one jig cut guide groove, wherein a jig cut guide groove comprises at least one selected from the group consisting of: a wedge shaped and dorsal, a straight linear shaped and medial, and a fully through a dorsal surface.

Still yet another embodiment of the disclosure is directed to a method for utilizing a bone cut to achieve correction of deformities having an intermetatarsal angle, the method comprising: cutting a metatarsal bone in a midshaft or proximal aspect of the metatarsal bone; fitting a first bone cut jig to the dimension and contour of a dorsal and medial surface of the metatarsal of the bone; inserting at least one support disposed such that it secures the first bone cut jig to the bone; fitting a jig cut guide groove in the bone cut jig for placement of a cutting tool, wherein the cutting tool traverses the jig cut guide groove, thereby creating the bone cut through the metatarsal from medial to lateral, wherein the bone cut is oriented from dorsal distal to plantar proximal; utilizing a plurality of dorsal holes disposed in a dorsal surface of the first bone cut jig for securing the first bone cut jig to the bone, thereby using at least one support, oriented dorsal to plantar for temporarily holding the first bone cut jig to the dorsal surface of the metatarsal, wherein at least one of the plurality of dorsal holes is proximal and at least one of the plurality of dorsal holes is distal to the bone cut, dorsal exit point; utilizing a plurality of medial surface holes disposed in a medial surface of the first bone cut jig for securing the first bone cut jig to the bone, thereby using at least one support for temporarily holding the first bone cut jig to the medial surface of the metatarsal, wherein at least one of the plurality of medial surface holes is superior proximal and at least one of the plurality is distal inferior to the jig cut guide groove; utilizing a bone cut IM correction clamp placed on the dorsal surface of the metatarsal after the bone cut is completed and a rotation axis support is placed, thereby as the bone cut IM correction clamp is closed down the metatarsal is rotated laterally closing down or reducing the intermetatarsal (IM) angle, thus substantially correcting the deformities; fitting a bone cut fixation plate for placing over the dorsal surface of the metatarsal to fix the bone cut rigidly, wherein the bone cut fixation plate has a slight bend distally to maintain a position dorsal on a distal segment of the bone as the intermetatarsal (IM) angle is reduced; and utilizing a plurality of fixation fasteners for securing the bone cut fixation plate permanently via dorsal holes disposed in a dorsal surface of the bone cut fixation plate such that it secures the bone cut fixation plate to the dorsal surface of the metatarsal, wherein at least one support is used, thereby temporarily securing the bone cut fixation plate proximal and distal as the plurality of fixation fasteners are being inserted.

A further embodiment of the disclosure is directed to the method further comprising: fitting a second bone cut jig dorsal and medial over a head of a metatarsal, wherein the second bone cut jig faces dorsal and medial; utilizing a plurality of fixation holes disposed in a dorsal surface and a medial surface of the second bone cut jig for temporarily stabilizing the second bone cut jig on the head of the metatarsal; using a plurality of supports for utilizing with the plurality of fixation holes, thereby temporarily stabilizing the second bone cut jig to the head of the metatarsal; and utilizing at least one jig cut guide groove, wherein a jig cut guide groove comprises at least one selected from the group consisting of: a wedge shaped and dorsal, a straight linear shaped and medial, and a fully through a dorsal surface.

### BRIEF DESCRIPTION OF THE DRAWINGS

FIG. 1A depicts a ROSE osteotomy jig from a top, right side view, utilized in a bunionectomy procedure according to the present disclosure.

FIG. 1B depicts a top view of the ROSE osteotomy jig of FIG. 1A.

FIG. 1C depicts a right side planar view of the ROSE osteotomy jig of FIG. 1A.

FIG. 2A is a diagram of a ROSE osteotomy IM correction clamp, utilized in a bunionectomy procedure according to the present disclosure.

FIG. 2B is a diagram of a ROSE osteotomy IM correction clamp on a metatarsal, utilized in a bunionectomy procedure according to the present disclosure.

FIG. 3A depicts a top plan view of a ROSE osteotomy fixation plate, utilized in a bunionectomy procedure according to the present disclosure.

FIG. 3B depicts a top, right side perspective view of the ROSE osteotomy fixation plate of FIG. 3A.

FIG. 4 is a diagram of a Reverdin osteotomy jig, utilized in a bunionectomy procedure according to the present disclosure.

FIG. 5A depicts a perspective view of a ROSE Osteotomy Jig with a rotational wire guide, alignment mark, and osteotomy cut guide, utilized in a ROSE bunionectomy procedure according to the present disclosure.

FIGS. 5B and 5C depict a right side and left side, perspective views of a ROSE Osteotomy Jig with a fixation wire insertion hole and positioning features, utilized in a ROSE bunionectomy procedure according to the present disclosure.

FIG. 6A depicts an isometric view of a ROSE Osteotomy Jig on a left first metatarsal, prior to an osteotomy cut, with a rotational K-wire partially drilled, utilized in a ROSE bunionectomy procedure according to the present disclosure.

FIG. 6B depicts a medial view of a ROSE Osteotomy Jig on a left first metatarsal, prior to an osteotomy cut, with a rotational K-wire partially drilled, utilized in a ROSE bunionectomy procedure according to the present disclosure.

FIG. 6C depicts a medial view of a ROSE Osteotomy Jig on a left first metatarsal, post osteotomy cut, with a rotational K-wire fully drilled, utilized in a ROSE bunionectomy procedure according to the present disclosure.

FIG. 7A depicts a perspective view of a ROSE Osteotomy Guide Clamp with handles open, prior to clamping, utilized in a ROSE bunionectomy procedure according to the present disclosure.

FIG. 7B depicts a cross-sectional view of a spring loaded detent used in a ROSE Osteotomy Guide Clamp, utilized in a ROSE bunionectomy procedure according to the present disclosure.

FIG. 7C depicts a ROSE Osteotomy Guide Clamp with handles in a clamping position, utilized in a ROSE bunionectomy procedure according to the present disclosure.

FIG. 7D depicts a ROSE Osteotomy Guide Clamp with handles in an Osteotomy cut clearance position, utilized in a ROSE bunionectomy procedure according to the present disclosure.

FIG. 7E depicts a ROSE Osteotomy Guide Clamp with handles in an alternative clearance position, utilized in a ROSE bunionectomy procedure according to the present disclosure.

FIG. 8A depicts a ROSE Osteotomy Guide Clamp positioned on the first metatarsal with handles in a clamping position, in preparation for insertion of a partially drilled K-wire, utilized in a ROSE bunionectomy procedure according to the present disclosure.

FIG. 8B depicts a ROSE Osteotomy Guide Clamp positioned on the first metatarsal with handles in a clamping position, with insertion of a partially drilled K-wire, utilized in a ROSE bunionectomy procedure according to the present disclosure.

FIG. 8C depicts a ROSE Osteotomy Guide Clamp with handles in an Osteotomy cut clearance position, clamped over the first metatarsal with insertion of a partially drilled K-wire in preparation for an Osteotomy cut, utilized in a ROSE bunionectomy procedure according to the present disclosure.

FIGS. 8D and 8E depict a full view and close up view of a ROSE Osteotomy Guide Clamp with handles in an Osteotomy cut clearance position, clamped over the first metatarsal with insertion of a partially drilled K-wire with a completed Osteotomy cut, utilized in a ROSE bunionectomy procedure according to the present disclosure.

FIG. 8F depicts a ROSE Osteotomy Guide Clamp with handles in a clamping position, with clamped metatarsal fragments in preparation for insertion of a fully drilled K-wire, utilized in a ROSE bunionectomy procedure according to the present disclosure.

FIG. 8G depicts a ROSE Osteotomy Guide Clamp with handles in a clamping position, clamped over the first metatarsal with a fully drilled K-wire and clamped metatarsal fragments, utilized in a ROSE bunionectomy procedure according to the present disclosure.

FIG. 9A depicts a ROSE Intermetatarsal (IM) Correction Clamp with an IM correction dial and attached osteotomy compression components, utilized in a ROSE bunionectomy procedure according to the present disclosure.

FIG. 9B depicts a below view of a ROSE Intermetatarsal (IM) Correction Clamp with an IM correction dial and attached osteotomy compression components, utilized in a ROSE bunionectomy procedure according to the present disclosure.

FIG. 9C depicts an exploded view of a ROSE Intermetatarsal (IM) Correction Clamp with an IM correction dial and attached osteotomy compression components, utilized in a ROSE bunionectomy procedure according to the present disclosure.

FIG. 9D depicts a ROSE Intermetatarsal (IM) Correction Clamp, which is clamped to the left first metatarsal dorsal fragment, with an IM correction dial and an osteotomy compression subassembly prepared for correction, utilized in a ROSE bunionectomy procedure according to the present disclosure.

FIG. 9E depicts a ROSE Intermetatarsal (IM) Correction Clamp, which is prepared with a swivel tip of an IM correction dial in contact with the medial face of the first metatarsal plantar fragment and an osteotomy compression subassembly, in light contact with a proximal plantar section of the plantar fragment, utilized in a ROSE bunionectomy procedure according to the present disclosure.

FIG. 9F depicts a ROSE Intermetatarsal (IM) Correction Clamp with a completed ROSE correction, whereby an IM correction dial contacts and rotates a plantar fragment around a K-wire guide, utilized in a ROSE bunionectomy procedure according to the present disclosure.

FIGS. 9G and 9H depict a below view and side view of a ROSE Intermetatarsal (IM) Correction Clamp with a completed ROSE correction, whereby an osteotomy compression subassembly secures the dorsal and plantar fragments, utilized in a ROSE bunionectomy procedure according to the present disclosure.

FIG. 9I depicts a ROSE Intermetatarsal (IM) Correction Clamp with a completed ROSE correction and a secured osteotomy compression subassembly, wherein a K-wire is removed and replaced with a compression screw, utilized in a ROSE bunionectomy procedure according to the present disclosure.

FIGS. 10A and 10B depict a top view and bottom view of a ROSE Fixation Clamp with mating bosses and contoured features to securely mate with slot and boss features of bone plate, as well as typical anatomic bone features and contours, utilized in a ROSE bunionectomy procedure according to the present disclosure.

FIG. 10C depicts a ROSE Fixation Clamp with boss features on the underside of the Fixation Clamp's dorsal pads mating with slots in the bone plate, for both dorsal and DP bone plate designs, utilized in a ROSE bunionectomy procedure according to the present disclosure.

FIG. 10D depicts a ROSE Fixation Clamp with clearance features on the Fixation Clamp's plantar pad, mating with the plantar boss feature of the DP bone plate, utilized in a ROSE bunionectomy procedure according to the present disclosure.

FIG. 10E depicts a ROSE Fixation Clamp is secured on a metatarsal, wherein the bone plate is fixated utilizing integrated screw guides and/or threaded features to mate with standard drill guides, utilized in a ROSE bunionectomy procedure according to the present disclosure.

FIG. 11A depicts a top view of a Reverdin Jig, utilized in a ROSE bunionectomy procedure and/or Reverdin correction procedure according to the present disclosure.

FIG. 11B depicts a bottom view of a Reverdin Jig, utilized in a ROSE bunionectomy procedure and/or Reverdin correction procedure according to the present disclosure.

FIG. 11C depicts a perspective view of a Reverdin Jig fixated to the left first metatarsal, wherein interaction with a DP plate and blade placements, utilized in a ROSE bunionectomy procedure and/or Reverdin correction procedure according to the present disclosure.

FIG. 11D depicts a top view of a Reverdin Jig fixated to the left first metatarsal, wherein interaction with a DP plate and blade placements, utilized in a ROSE bunionectomy procedure and/or Reverdin correction procedure according to the present disclosure.

FIG. 11E depicts a right side view of a Reverdin Jig fixated to the left first metatarsal, wherein interaction with a DP plate and a medial blade placement, utilized in a ROSE bunionectomy procedure and/or Reverdin correction procedure according to the present disclosure.

FIG. 11F depicts a right side view of a Reverdin Jig fixated to the left first metatarsal, wherein interaction with a DP plate and both medial and dorsal blade placements, utilized in a ROSE bunionectomy procedure and/or Reverdin correction procedure according to the present disclosure.

FIG. 12A depicts a left side, perspective view of a Reverdin Guide Clamp with handles in a clamping position and with an integrated Reverdin Jig, utilized in a ROSE bunionectomy procedure and/or Reverdin correction procedure according to the present disclosure.

FIGS. 12B and 12C depict a front and side perspective views of a Reverdin Guide Clamp with handles in a cut clearance (a.k.a., “folded”) position and with an integrated Reverdin Jig, utilized in a ROSE bunionectomy procedure and/or Reverdin correction procedure according to the present disclosure.

FIGS. 12D and 12E depict a top, right side and left side perspective views of a Reverdin Guide Clamp with handles in a clamping position and with an integrated Reverdin Jig, utilized in a ROSE bunionectomy procedure and/or Reverdin correction procedure according to the present disclosure.

FIG. 12F depicts a Reverdin Guide Clamp with handles in a cut clearance position and with an integrated Reverdin Jig, utilized in a ROSE bunionectomy procedure and/or Reverdin correction procedure according to the present disclosure.

FIG. 12G depicts a side view of a Reverdin Guide Clamp utilized in the Reverdin procedure steps and with an integrated Reverdin Jig, utilized in a ROSE bunionectomy procedure and/or Reverdin correction procedure according to the present disclosure.

FIG. 12H depicts a left side, perspective view of a Reverdin Guide Clamp utilized in the Reverdin procedure steps and with an integrated Reverdin Jig, utilized in a ROSE bunionectomy procedure and/or Reverdin correction procedure according to the present disclosure.

FIG. 12I depicts a front, side view of a Reverdin Guide Clamp utilized in the Reverdin procedure steps and with an integrated Reverdin Jig, utilized in a ROSE bunionectomy procedure and/or Reverdin correction procedure according to the present disclosure.

FIGS. 13A and 13B depict a top and bottom perspective views of a dorsal bone plate having a marking on the dorsal surface used to align the bone plate with an osteotomy cut, utilized in a ROSE bunionectomy procedure according to the present disclosure.

FIGS. 13C and 13D depict a top and bottom perspective views of a dorsal bone plate secured to the dorsal surface of a corrected metatarsal, utilized in a ROSE bunionectomy procedure according to the present disclosure.

FIGS. 14A and 14B depict a top and bottom perspective views of a dorsal plantar (DP) bone plate having a marking on the dorsal surface used to align the bone plate with an osteotomy cut and an integrated internally threaded boss to accept a clamping screw, utilized in a ROSE bunionectomy procedure according to the present disclosure.

FIG. 14C depicts a top, perspective view of a dorsal plantar (DP) bone plate having a marking on the DP bone plate, used to align the bone plate with an Osteotomy cut, utilized in a ROSE bunionectomy procedure according to the present disclosure.

FIG. 14D depicts a bottom, perspective view of a fixated dorsal plantar (DP) bone plate having a marking on the DP bone plate with a clamping screw installed within an internally threaded boss, utilized in a ROSE bunionectomy procedure according to the present disclosure.

FIG. 15A depicts a perspective view of a ROSE Osteotomy Correction Jig device, utilized in a ROSE bunionectomy procedure according to the present disclosure.

FIG. 15B depicts a front view of a ROSE Osteotomy Correction Jig device, utilized in a ROSE bunionectomy procedure according to the present disclosure.

FIG. 15C depicts a rear view of a ROSE Osteotomy Correction Jig device, utilized in a ROSE bunionectomy procedure according to the present disclosure.

FIG. 15D depicts an exploded, front view of a ROSE Osteotomy Correction Jig device, utilized in a ROSE bunionectomy procedure according to the present disclosure.

FIG. 15E depicts a perspective view of a ROSE Osteotomy Correction Jig device positioned on a left first metatarsal, utilized in a ROSE bunionectomy procedure according to the present disclosure.

FIG. 15F depicts a perspective view of a ROSE Osteotomy Correction Jig device positioned on a left first metatarsal and secured with threaded olive wires in preparation for an Osteotomy, utilized in a ROSE bunionectomy procedure according to the present disclosure.

FIG. 15G, depicts a medial view of a ROSE Osteotomy Correction Jig device positioned on a left first metatarsal, secured with threaded olive wires, and after an Osteotomy, utilized in a ROSE bunionectomy procedure according to the present disclosure.

FIG. 15H depicts a medial view of a ROSE Osteotomy Correction Jig device positioned on a left first metatarsal, clamped with rotation K-wire installed and distal wires removed in preparation for bone correction, utilized in a ROSE bunionectomy procedure according to the present disclosure.

FIG. 15I depicts a medial view of a ROSE Osteotomy Correction Jig device positioned on a left first metatarsal, clamped with rotation K-wire installed and proximal wires removed with an 18 degree bone correction, utilized in a ROSE bunionectomy procedure according to the present disclosure.

FIG. 15J depicts a medial view of a ROSE Osteotomy Correction Jig device positioned on a left first metatarsal, clamped with rotation K-wire installed and proximal wires removed with an 18 degree bone correction, retained with a compression screw, utilized in a ROSE bunionectomy procedure according to the present disclosure.

FIG. 16A depicts a lateral view of a ROSE Bunion Correction system and method utilizing a Mini ROSE technique for a fifth metatarsal, Tailors bunion deformity oriented oblique osteotomy from dorsal distal to plantar proximal utilized in a ROSE bunionectomy procedure according to the present disclosure.

FIG. 16B depicts a perspective view of a ROSE Bunion Correction system and method utilizing a Mini ROSE technique for a fifth metatarsal, Tailors bunion deformity oriented oblique osteotomy from dorsal distal to plantar proximal, positioned and secured on a fifth metatarsal with a Mini ROSE Osteotomy Correction Jig assembly, utilized in a ROSE bunionectomy procedure according to the present disclosure.

FIG. 16C depicts a front view of a Mini ROSE (a.k.a., ROSE Mini) Osteotomy Correction Jig assembly, utilized in a ROSE bunionectomy procedure according to the present disclosure.

FIG. 16D depicts a rear view of a Mini ROSE (a.k.a., ROSE Mini) Osteotomy Correction Jig assembly, utilized in a ROSE bunionectomy procedure according to the present disclosure.

FIG. 16E depicts an exploded, front view of a Mini ROSE (a.k.a., ROSE Mini) Osteotomy Correction Jig assembly, utilized in a ROSE bunionectomy procedure according to the present disclosure.

FIG. 16F depicts a perspective view of a ROSE Bunion Correction system and method utilizing a Mini ROSE technique for a fifth metatarsal, Tailors bunion deformity oriented oblique osteotomy from dorsal distal to plantar proximal, positioned on the left fifth metatarsal with a Mini ROSE (a.k.a., ROSE Mini) Osteotomy Correction Jig assembly, utilized in a ROSE bunionectomy procedure according to the present disclosure.

FIG. 16G depicts a perspective view of a ROSE Bunion Correction system and method utilizing a Mini ROSE technique for a fifth metatarsal, Tailors bunion deformity oriented oblique osteotomy from dorsal distal to plantar proximal, positioned and secured by threaded olive wires on a fifth metatarsal with a Mini ROSE (a.k.a., ROSE Mini) Osteotomy Correction Jig assembly in preparation for an Osteotomy, utilized in a ROSE bunionectomy procedure according to the present disclosure.

FIG. 16H depicts a medial view of a ROSE Bunion Correction system and method utilizing a Mini ROSE technique for a fifth metatarsal, Tailors bunion deformity oriented oblique osteotomy from dorsal distal to plantar proximal, positioned and secured by threaded olive wires on the left fifth metatarsal, and after the Osteotomy with a Mini ROSE (a.k.a., ROSE Mini) Osteotomy Correction Jig assembly, utilized in a ROSE bunionectomy procedure according to the present disclosure.

FIG. 16I depicts a medial view of a ROSE Bunion Correction system and method utilizing a Mini ROSE technique for a fifth metatarsal, Tailors bunion deformity oriented oblique osteotomy from dorsal distal to plantar proximal, after the Osteotomy, and clamped with a rotation K-wire installed and distal wires removed, in preparation for bone correction with a Mini ROSE (a.k.a., ROSE Mini) Osteotomy Correction Jig assembly, utilized in a ROSE bunionectomy procedure according to the present disclosure.

FIG. 16J depicts a right side, perspective view of a ROSE Bunion Correction system and method utilizing a Mini ROSE technique for a fifth metatarsal, Tailors bunion deformity oriented oblique osteotomy from dorsal distal to plantar proximal, after the Osteotomy, clamped with a rotation K-wire installed and distal wires removed, with bone correction, and with a Mini ROSE (a.k.a., ROSE Mini) Osteotomy Correction Jig assembly, utilized in a ROSE bunionectomy procedure according to the present disclosure.

FIG. 16K depicts a medial view of a ROSE Bunion Correction system and method utilizing a Mini ROSE technique for a fifth metatarsal, Tailors bunion deformity oriented oblique osteotomy from dorsal distal to plantar proximal, after the Osteotomy, with wires removed, with bone correction retained with a compression screw, and with a Mini ROSE (a.k.a., ROSE Mini) Osteotomy Correction Jig assembly, utilized in a ROSE bunionectomy procedure according to the present disclosure.

FIG. 16L depicts a medial view of a ROSE Bunion Correction system and method utilizing a Mini ROSE technique for a fifth metatarsal, Tailors bunion deformity oriented oblique osteotomy from dorsal distal to plantar proximal, after the Osteotomy, with bone correction fully fixated with a dorsal plate and locking or non-locking screws, utilized in a ROSE bunionectomy procedure according to the present disclosure.

FIG. 16M depicts a medial view of a ROSE Bunion Correction system and method utilizing a Mini ROSE technique for a fifth metatarsal, Tailors bunion deformity oriented oblique osteotomy from dorsal distal to plantar proximal, after the Osteotomy, with bone correction fully fixated with a dorsal plantar (DP) plate, clamping screw and locking or non-locking screws, utilized in a ROSE bunionectomy procedure according to the present disclosure.

FIG. 17A depicts a lateral view of a ROSE Bunion Correction system and method utilizing a ROSE D technique, Ludloff Osteotomy oriented oblique osteotomy from dorsal proximal to plantar distal, utilized in a ROSE bunionectomy procedure according to the present disclosure.

FIG. 17B depicts a lateral view of a ROSE Bunion Correction system and method utilizing a ROSE D technique, Ludloff Osteotomy oriented oblique osteotomy from dorsal proximal to plantar distal positioned and secured on a first metatarsal with a ROSE-D (a.k.a., Ludloff) Osteotomy Correction Jig assembly/system in preparation for a Ludloff Osteotomy, utilized in a ROSE bunionectomy procedure according to the present disclosure.

FIG. 18A depicts a right side, perspective view of an Osteotomy Guide Clamp with rotationally folding handles, utilized in a ROSE bunionectomy procedure according to the present disclosure.

FIG. 18B depicts a right side, perspective view of an Osteotomy Guide Clamp with rotationally folding handles with jig feature mounted perpendicular to the lateral plane of the exposed first metatarsal, utilized in a ROSE bunionectomy procedure according to the present disclosure.

FIG. 18C depicts a left side, perspective view of an Osteotomy Guide Clamp with rotationally folding handles with jig feature mounted perpendicular to the lateral plane of an exposed first metatarsal with handles rotationally folded towards the distal end of the metatarsal and retained by the distal detent position, utilized in a ROSE bunionectomy procedure according to the present disclosure.

FIG. 19A depicts a front side, perspective view of a Reverdin Clamp with rotationally folding handles with jig feature mounted perpendicular to the lateral plane of the head of an exposed first metatarsal, utilized in a ROSE bunionectomy procedure is shown, according to the present disclosure.

FIG. 19B depicts a right side, perspective view of a Reverdin Clamp with rotationally folding handles with jig feature mounted perpendicular to the lateral plane of the head of an exposed first metatarsal with handles rotationally folded towards the proximal end of the metatarsal and retained by the proximal detent position, utilized in a ROSE bunionectomy procedure according to the present disclosure.

FIG. 20A depicts a dorsal side, perspective view of a curved bone plate that securely fixates the dorsal and plantar fragments of an exposed first metatarsal, utilized in a ROSE bunionectomy procedure according to the present disclosure.

FIG. 20B depicts a plantar side, perspective view of a curved bone plate that securely fixates the dorsal and plantar fragments of an exposed first metatarsal, utilized in a ROSE bunionectomy procedure according to the present disclosure.

FIG. 20C depicts a right side, perspective view of a curved bone that securely fixates the dorsal and plantar fragments of an exposed first metatarsal, utilized in a ROSE bunionectomy procedure according to the present disclosure.

FIG. 21A depicts a right side, perspective view of a correction clamp that rotates the first metatarsal bone around an axis pin to reduce or decrease the first intermetatarsal angle, utilized in a ROSE bunionectomy procedure according to the present disclosure.

FIGS. 21B, 21C, and 21D depict perspective views of a correction clamp rotating the first metatarsal bone around an axis pin to reduce or decrease the first intermetatarsal angle, utilized in a ROSE bunionectomy procedure according to the present disclosure.

FIG. 22A depicts a perspective view of an alternative embodiment of a dorsal bone plate with mating feature for compression screw, utilized in a ROSE bunionectomy procedure according to the present disclosure.

FIG. 22B depicts a top, perspective view of an alternative embodiment of a dorsal bone plate with mating feature for compression screw fixated to a corrected metatarsal, utilized in a ROSE bunionectomy procedure according to the present disclosure.

FIG. 23A depicts a top, perspective view of an alternative embodiment of a dorsal plantar (DP) plate with mating feature for compression screw, utilized in a ROSE bunionectomy procedure according to the present disclosure.

FIG. 23B depicts a side, perspective view of an alternative embodiment of a dorsal plantar (DP) plate with mating feature for compression screw, utilized in a ROSE bunionectomy procedure according to the present disclosure.

FIG. 23C depicts a top, perspective view of an alternative embodiment of a dorsal plantar (DP) plate with mating feature for compression screw fixated to a corrected metatarsal, utilized in a ROSE bunionectomy procedure according to the present disclosure.

FIG. 23D depicts a side, perspective view of an alternative embodiment of a dorsal plantar (DP) plate with mating feature for compression screw fixated to a corrected metatarsal, utilized in a ROSE bunionectomy procedure according to the present disclosure.

FIGS. 24A and 24B depict a perspective and cross-sectional views of a cannulated dorsal plantar (DP) screw (i.e., clamping screw), utilized in a ROSE bunionectomy procedure according to the present disclosure.

FIGS. 24C and 24D depict perspective and cross-sectional views of a dorsal plantar (DP) bone plate (i.e., DP plate) and cannulated dorsal plantar (DP) screw (i.e., clamping screw) having a lower threaded portion of the clamping screw mate with an internally threaded boss of the DP plate to effect a clamping force, utilized in a ROSE bunionectomy procedure according to the present disclosure.

A component or a feature that is common to more than one drawing is indicated with the same reference number in each of the drawings.

### DETAILED DESCRIPTION OF THE PREFERRED EMBODIMENTS

The system and method described herein include but are not limited to the metatarsal bones and/or joints (e.g., the first metatarsal bone and/or the fifth metatarsal bone, respectively). For this disclosure below, reference will be made to include the “metatarsal” and the “first metatarsal” but are not limited to the foregoing references (e.g., the fifth metatarsal).

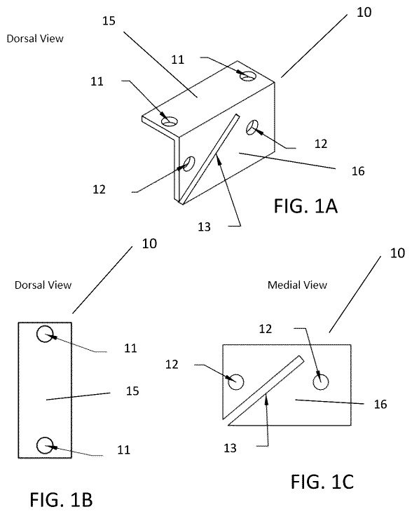

In FIG. 1A, a diagram of a ROSE osteotomy jig generally represented by reference numeral 10 and utilized in a bunionectomy procedure is shown, according to the present disclosure. The ROSE osteotomy jig 10 is manufactured to fit the dimensions and contours of the dorsal and medial surface of the first and/or fifth metatarsal.

Referring to FIG. 1A, the ROSE osteotomy jig 10 (hereinafter “jig 10”) may comprise a plurality of dorsal holes 11, a plurality of medial surface holes 12, a jig cut guide groove 13, at least one wire 14 (e.g., olive wire, K-wire, and/or guidewire, etc.), a dorsal surface 15, a medial surface 16, and a rotation axis wire 17 (e.g., olive wire, K-wire, and/or guidewire, etc.), wherein the plurality of dorsal holes 11 are shown for at least one wire 14 (not shown) insertion. Further, at least one wire 14 temporarily holds the jig 10 to a dorsal surface of a first metatarsal (not shown), e.g., one proximal dorsal holes 11 and one distal dorsal holes 11 to the osteotomy dorsal exit point (not shown). Thus, at least one wire 14 is oriented dorsal to plantar. The proximal dorsal holes 11 will become the hole for placement of the rotation axis wire 17 (not shown).

Referring to FIGS. 1A-C, the ROSE osteotomy jig 10 may comprise a plurality of medial surface holes 12 for at least one wire 14 insertion. Further, at least one wire 14 temporarily holds the jig 10 in place on a medial surface of a first metatarsal (not shown), e.g., one superior proximal medial surface holes 12 to the jig cut guide groove 13 and one distal inferior medial surface holes 12 to the jig cut guide groove 13.

Referring to FIGS. 1A and 1C, the ROSE osteotomy jig 10 may comprise a jig cut guide groove 13. Further, this is where a cutting tool including but not limited to a saw blade (not shown) fits in the jig cut guide groove 13 to create a ROSE osteotomy cut (not shown) through the first metatarsal (not shown) from medial to lateral. The ROSE osteotomy cut is oriented from dorsal distal to plantar proximal. This leaves a short dorsal first metatarsal surface (not shown) and a longer plantar first metatarsal surface including the head (not shown). The dorsal first metatarsal surface prevents elevation and malalignment of the first metatarsal. In some embodiments, a secondary wire 14 (not shown) can be inserted to help maintain the corrected position while fixating the ROSE osteotomy.

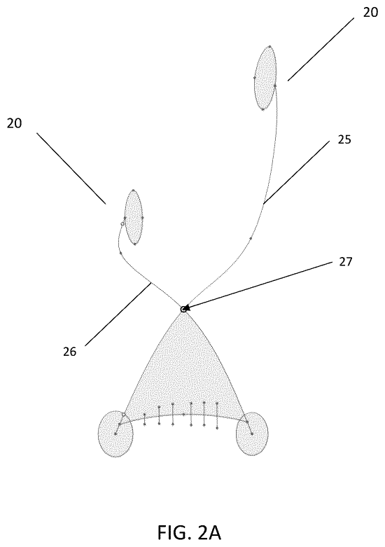

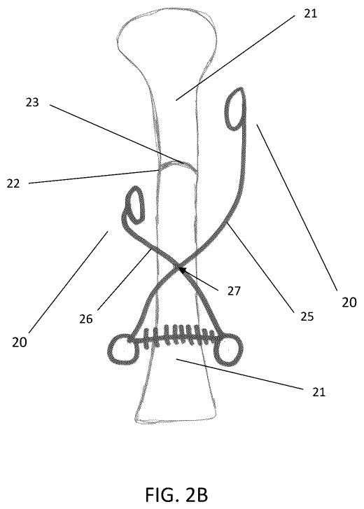

In FIG. 2A, a diagram of a ROSE osteotomy IM correction clamp generally represented by reference numeral 20 and utilized in a bunionectomy procedure is shown, according to the present disclosure. The ROSE osteotomy IM correction clamp 20 is manufactured to close down or reduce the IM (intermetatarsal) angle.

Referring to FIG. 2A, the ROSE osteotomy IM correction clamp 20 (hereinafter “clamp 20”) may comprise a dorsal surface of a first metatarsal 21, a ROSE osteotomy cut 22, an intermetatarsal (IM) angle 23, and a rotation axis wire 27 (e.g., olive wire, K-wire, and/or guidewire, etc.), wherein the clamp 20 is placed on the dorsal surface of the first metatarsal 21 (not shown) after the ROSE osteotomy cut 22 (not shown) is completed and the rotation axis wire 27 is in place. As the clamp 20 is closed down the first metatarsal 21 rotates laterally closing down or reducing the IM (intermetatarsal) angle 23 (not shown), thus correcting the deformity. Each click of the clamp 20 closing, dials down further IM angle 23 reduction. The longer arm 25 is distal medial to the dorsal surface of the cut and the shorter arm 26 is proximal lateral to the dorsal surface of the cut. In some embodiments, a secondary wire 24 can be inserted to help maintain the corrected position while fixating the ROSE osteotomy. In further embodiments, the clamp 20 may comprise a longer proximal lateral arm and a shorter distal medial arm to account for the left and right foot, respectively.

In FIG. 2B, a diagram of a ROSE osteotomy IM correction clamp generally represented by reference numeral 20 and utilized in a bunionectomy procedure is shown, according to the present disclosure. The ROSE osteotomy IM correction clamp 20 is manufactured to close down or reduce the IM (intermetatarsal) angle.

Referring to FIG. 2B, the ROSE osteotomy IM correction clamp 20 (hereinafter “clamp 20”) may comprise a dorsal surface of a first metatarsal 21, a ROSE osteotomy cut 22, an intermetatarsal (IM) angle 23, and a rotation axis wire 27 (e.g., olive wire, K-wire, and/or guidewire, etc.), wherein the clamp 20 is placed on the dorsal surface of the first metatarsal 21 after the ROSE osteotomy cut 22 is completed and the rotation axis wire 27 is in place. As the clamp 20 is closed down the first metatarsal 21 rotates laterally closing down or reducing the IM (intermetatarsal) angle 23, thus correcting the deformity. Each click of the clamp 20 closing, dials down further IM angle 23 reduction. The longer arm 25 is distal medial to the dorsal surface of the cut and the shorter arm 26 is proximal lateral to the dorsal surface of the cut. In some embodiments, a secondary wire 24 (not shown) can be inserted to help maintain the corrected position while fixating the ROSE osteotomy. In further embodiments, the clamp 20 may comprise a longer proximal lateral arm and a shorter distal medial arm to account for the left and right foot, respectively.

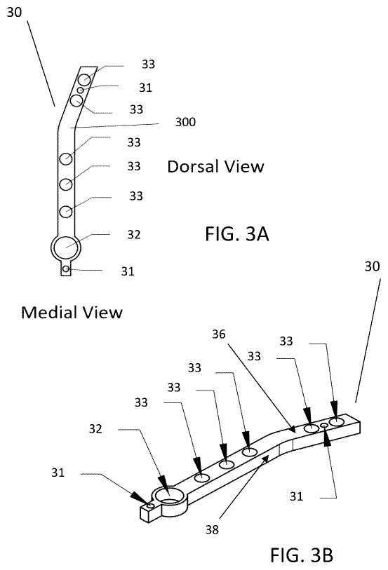

In FIGS. 3A and 3B, a diagram of a ROSE osteotomy fixation plate generally represented by reference numeral 30 and utilized in a bunionectomy procedure is shown, according to the present disclosure. The ROSE osteotomy fixation plate 30 is manufactured to fix the osteotomy rigidly. It has a slight bend distally to maintain the position dorsal on the distal segment as the IM angle is reduced. Further, a compression and/or locking or non-locking screw may comprise a range of a standard diameter utilized according to procedural specifications.

Referring to FIGS. 3A and 3B, the ROSE osteotomy fixation plate 30 (hereinafter “fixation plate 30”) may comprise a plurality of wire holes 31, at least one hole fitting 32, a plurality of screw holes 33, at least one compression screw 35 (not shown), a dorsal surface 36, a medial surface 38, a plurality of locking or non-locking screws 39 (not shown), and at least one wire 34 (e.g., olive wire, K-wire, and/or guidewire, etc.) (not shown), wherein the plurality of wire holes 31 are shown for temporarily securing the fixation plate 30 proximal and distal with at least one wire 34, as the compression screw 35 and locking or non-locking screws 39 are being inserted.

Referring to FIGS. 3A and 3B, the ROSE osteotomy fixation plate 30 may comprise at least one compression screw 35, which is inserted in at least one hole fitting 32 before the plate is secured on the bone. Further, the compression screw 35 may be a 3.5 mm diameter screw, which is placed in the at least one hole fitting 32 where the rotation axis wire 37 (not shown) was and uses that hole fitting 32 herein created by the rotation axis wire 37 as a guide hole for drilling for the compression screw 35. This compression screw 35 will be the first permanent rigid fixation point of the ROSE osteotomy. In some embodiments, the at least one hole fitting 32 may be slightly larger than 3.5 mm diameter and can fit over the compression screw 35. Thus, the concept of having this hole fitting 32 may rotate the fixation plate 30 about or around it. Or, in one embodiment, to have the 3.5 mm diameter screw be a compression screw 35 through the plate and/or sitting independently of the plate. Alternatively, in some embodiments, the hole fitting 32 may not be utilized, and the fixation plate 30 can begin with the first, proximal to the dorsal exit point of the ROSE osteotomy of the plurality of screw holes 33. Further, a compression and/or locking or non-locking screw may comprise a range of a standard diameter utilized according to procedural specifications.

Referring to FIGS. 3A and 3B, the ROSE osteotomy fixation plate 30 may comprise a plurality of screw holes 33, and a plurality of locking or non-locking screws 39. Further, the fixation plate 30 may comprise at least three 2.4 mm diameter locking or non-locking screws 39 (not shown) located proximal to the dorsal exit point of the ROSE osteotomy, and two 2.4 mm diameter locking or non-locking screws 39 (not shown) located at the distal to the dorsal exit point of the ROSE osteotomy. The solid portion 300 of the fixation plate 30 without holes lies over the dorsal exit point of the ROSE osteotomy. At this solid portion 300, the fixation plate 30 is angled or bent slightly lateral to remain dorsally placed on the distal aspect of the first metatarsal (not shown), which includes the head.

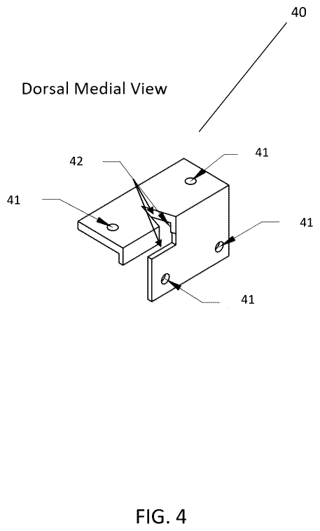

In FIG. 4, a diagram of a Reverdin osteotomy jig generally represented by reference numeral 40 and utilized in a bunionectomy procedure is shown, according to the present disclosure. The Reverdin osteotomy jig 40 is manufactured to fit dorsal and medial over the head of a first metatarsal. Thus, the Reverdin osteotomy jig 40 faces dorsal and medial.

Referring to FIG. 4, the Reverdin osteotomy jig 40 (hereinafter “jig 40”) may comprise a plurality of fixation holes 41, a plurality of wires 44 (e.g., olive wire, K-wire, and/or guidewire, etc.), wherein the plurality of fixation holes 41, by utilizing the plurality of wires 44 (not shown) for insertion, temporarily stabilize the jig 40 on the head of a first metatarsal 43 (not shown).

Referring to FIG. 4, the Reverdin osteotomy jig 40 may comprise at least one jig cut guide groove 42, wherein in some embodiments, two cut guide grooves 42 are at least one dorsal and one medial. Further, the dorsal may comprise a wedge shaped to allow for correction of the articular deviation at the head of the first metatarsal 43. The medial may comprise a straight linear cut oriented distal to proximal to protect the sesamoid apparatus. In some embodiments, the cut can also be fully through the dorsal surface to allow for some translation of the head of the first metatarsal 43 (not shown) for more IM angle 45 correction (not shown).

Therefore, in order to allow for consistent reliable reproduction of the osteotomy cut, rigid internal fixation to allow for early weightbearing, and adequate correction of the deformity to minimize recurrence, as well as, avoiding the complication of nonunion of the metatarsal cuneiform joint (i.e., the tarsometatarsal joint), the unique osteotomy jigs disclosed herein, IM correction bone clamp disclosed herein, and bone plate with screws disclosed herein, have been designed to achieve these goals and solve these technical problems.

### ROSE Bunion Correction Procedure:

The Rotational Osteotomy System and method for Enhanced Bunion Correction (ROSE) is a set of novel instruments and implants to repeatedly and robustly perform the novel osteotomy cut and bunion correction system and method developed by Dr. Tzvi Bar-David. The procedure may additionally benefit from Dr. Bar-David's novel instrument for Reverdin correction, a procedure that can be implemented as the optional final step of a ROSE correction, or as a stand-alone procedure.

The present disclosure describes herein the design features and benefits of the full set of ROSE instruments and implants including those uniquely associated with the Reverdin correction.

The novel instruments may comprise:
A ROSE Osteotomy Jig.
A ROSE Osteotomy Guide Clamp.
A ROSE Intermetatarsal (IM) Correction Clamp.
A ROSE Osteotomy-Correction Jig.
A Mini ROSE Osteotomy Jig.
A Mini ROSE Osteotomy-Correction Jig.
A ROSE-D Osteotomy Jig.
A Dorsal Extended Bone Plate.
A Dorsal Plantar (DP) Extended Plate.
A Clamping Screw.
A ROSE Fixation Clamp.
A Reverdin Jig.
A Reverdin Guide Clamp.
The novel implants may comprise:
A Dorsal Bone Plate.
A Dorsal Plantar (DP) Bone Plate.

The novel features of the ROSE procedure that may be used in other applications may comprise:
Additional Surgical Clamps with Folding Handles.

Additional details of the ROSE Bunion Correction:
Each of the ROSE instruments and implants has a left-hand and/or right-hand version.

The procedure may utilize additional instruments and implants already known in the arts including sagittal bone saws and blades, bone drills, Kirshner wires, olive wires, locking or non-locking bone screws, cannulated or non-cannulated bone screws, compression screws, and similar.

The terms “tool(s)” or “instrument(s)” or “device(s)” or “apparatus” or “design(s)” or “assembly,” or a combination thereof, are to be used interchangeably when interpreted as specifying the presence of stated inventions described herein, but not precluding the presence of one or more other inventions described herein or groups thereof.

### ROSE Osteotomy Jig

The ROSE bunionectomy procedure (i.e., ROSE Bunion Correction Procedure) provides benefits over other bunion correction protocols by increasing or reducing the first intermetatarsal angle without a change in overall metatarsal length and an optimized fixated fragment interface to assist in post ambulatory recovery.

These dual benefits are driven, in part, by the perpendicular relationship between the metatarsal dorsal and plantar fragment rotational axis and the osteotomy cut plane, which in turn becomes the contact interface between the fixated dorsal and plantar fragments. This optimized interface has a maximized surface area, thereby creating maximum bone contact between the dorsal and plantar metatarsal fragments for enhanced bone healing. Also, this interface is located on a plane that provides, once fixated, optimal support against typical post-ambulatory ground reaction forces in two ways. First, the osteotomy creates a stabilizing dorsal shelf to resist ground reaction forces. Second, the orientation of the plane of the osteotomy is essentially parallel to the weight bearing surface, thereby minimizing the effect of the ground reaction forces on the osteotomy itself.

The ROSE Bunion Correction system and method may comprise a ROSE Osteotomy Jig (hereinafter “ROSE Jig”). The ROSE Jig is designed to readily determine proper positioning of the osteotomy cut and relative location of the rotational axis. The jig may comprise positioning features geometrically designed to position with anatomic bone contours, fixation features, a slot feature to properly guide a sagittal saw blade fully through the metatarsal bone from a dorsal distal insertion to a plantar proximal exit without cartilage damage, and a guide feature to perpendicularly guide a Kirshner wire, or similar wire described herein, towards and/or through the osteotomy cut.

To utilize the ROSE Jig, the jig is first positioned using surgeon markings and/or anatomical features on the first metatarsal bone. This may include a mark applied by the surgeon to the mid-line dorsal surface of the exposed metatarsal bone prior to jig placement, which is then aligned with markings on the jig. Additionally, the jig may comprise organically designed bosses and surfaces to contact and mate with typical bone surface geometry.

Once properly placed, the jig can be fixated to the metatarsal bone in two medial locations by drilling olive wires, or similar wires, through each of two wire location holes in the near face of the jig. These fixation wires enter the jig, pass through the jig, and enter the medial face of the metatarsal. Countersinks on the near face of the jig, at the wire location holes, are intended to mate with olive-style orthopedic wires, which may be the preferred fixation wire type. The jig feature is now mounted flush against the medial side of the exposed first metatarsal.

With proper placement and fixation, the jig now clearly indicates and allows for proper positioning of the osteotomy cut and the placement of the rotational K-wire, or similar wire (e.g., olive wire, K-wire, and/or guidewire, etc.).

The ROSE Osteotomy Jig can be utilized as a stand-alone orthopedic instrument, and/or as incorporated into the ROSE Osteotomy Guide Clamp described herein.

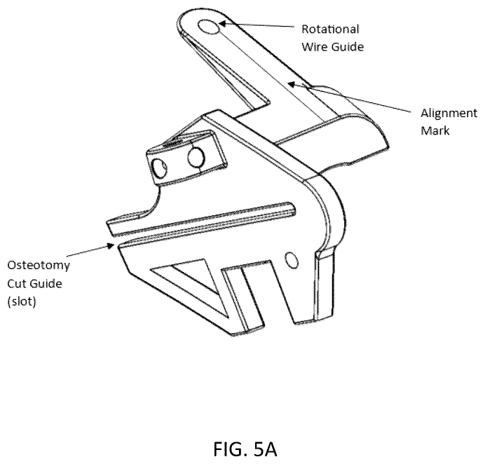

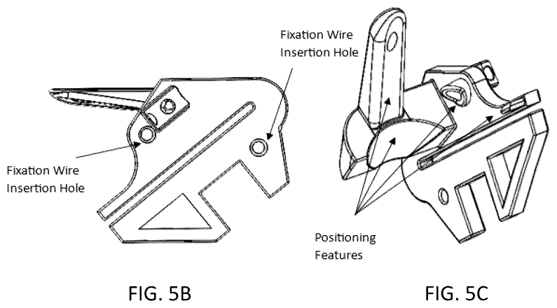

In FIG. 5A, a perspective view of a ROSE Osteotomy Jig with a rotational wire guide, alignment mark, and osteotomy cut guide generally represented by reference numeral 50 and utilized in a ROSE bunionectomy procedure is shown, according to the present disclosure. The ROSE Jig 50 may comprise an osteotomy cut guide (slot) 51, a rotational wire guide 52, and an alignment mark 53.

In FIGS. 5B and 5C, a right side and left side, perspective views of a ROSE Osteotomy Jig with a fixation wire insertion hole and positioning features generally represented by reference numeral 50 and utilized in a ROSE bunionectomy procedure are shown, according to the present disclosure. The ROSE Jig 50 may comprise fixation wires 54, a fixation wire insertion hole 55, and positioning features 56.

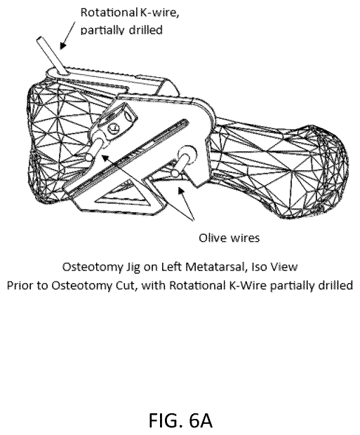

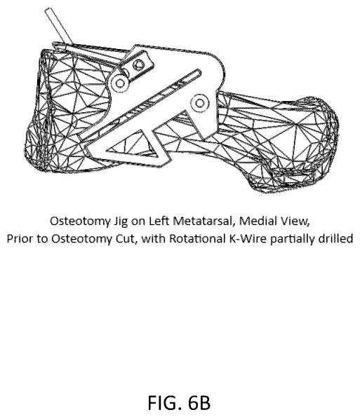

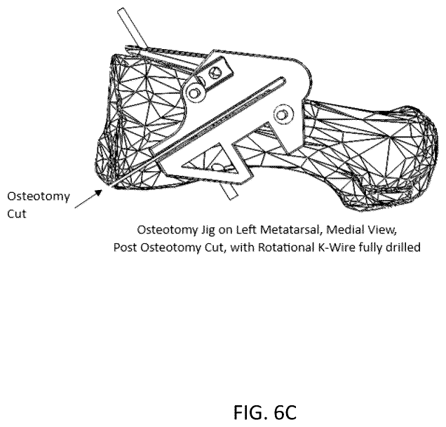

In FIG. 6A, an isometric view of a ROSE Osteotomy Jig on a left first metatarsal, prior to an osteotomy cut, with a rotational K-wire partially drilled generally represented by reference numeral 60 and utilized in a ROSE bunionectomy procedure is shown, according to the present disclosure. The ROSE Jig 60 may comprise olive wires 67, and a rotational K-wire 68.

In FIG. 6B, a medial view of a ROSE Osteotomy Jig on a left first metatarsal, prior to an osteotomy cut, with a rotational K-wire partially drilled generally represented by reference numeral 60 and utilized in a ROSE bunionectomy procedure is shown, according to the present disclosure. The ROSE Jig 60 may comprise olive wires 67, and a rotational K-wire 68.

In FIG. 6C, a medial view of a ROSE Osteotomy Jig on a left first metatarsal, post osteotomy cut, with a rotational K-wire fully drilled generally represented by reference numeral 60 and utilized in a ROSE bunionectomy procedure is shown, according to the present disclosure. The ROSE Jig 60 may comprise olive wires 67, a rotational K-wire 68, and an osteotomy cut 69.

### ROSE Osteotomy Guide Clamp

The ROSE Bunion Correction system and method may comprise a ROSE Osteotomy Guide Clamp (hereinafter “ROSE Guide Clamp”). The ROSE Guide Clamp instrument incorporates the ROSE Osteotomy Jig described herein with a unique hinged clamp for consistent and reliable rotational wire insertion, and reproduction of the ROSE osteotomy cut. The hinged clamp handles allow for robust clamping of the instrument to the metatarsal before, during and after the rotational wire insertion and/or osteotomy cut. These clamp handles can be rotationally repositioned (e.g., folded) to the distal or proximal location, providing sufficient clearance to allow the physician to make the osteotomy cut with a sagittal saw while the instrument remains clamped.

This folding motion is required to allow the handles and body of the clamp to be moved out of the way so that they don't interfere and get in the way of the saw as it is inserted in the jig and cuts the bone. This novel and unique feature of a hinge built into the clamp to enable the handles and body of the clamp to be repositioned in different directions to allow freedom from an intrinsic block, which would otherwise be caused by the handles and body of the clamp, has applications in surgical specialties of all disciplines.

In one embodiment of the Osteotomy Guide Clamp described herein, the rotational wire guide feature may comprise a threaded portion to accept a standard orthopedic, threaded drill guide (a.k.a., tower).

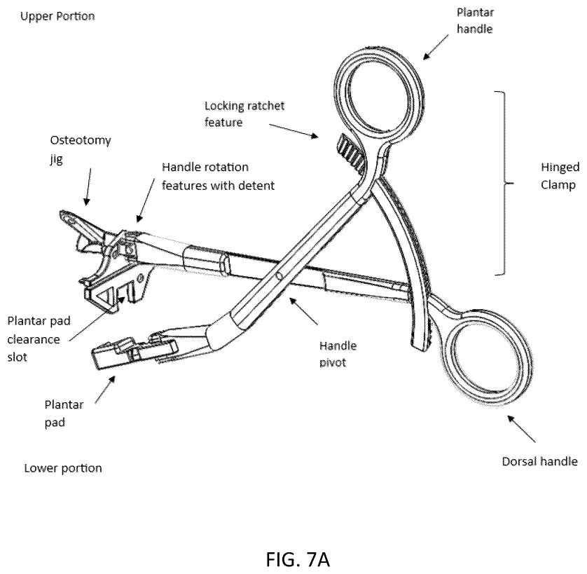

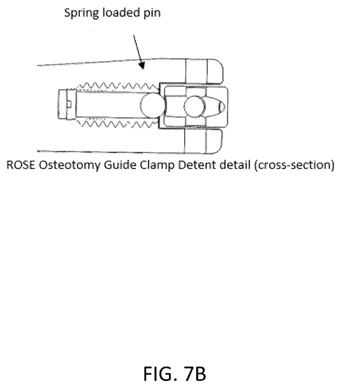

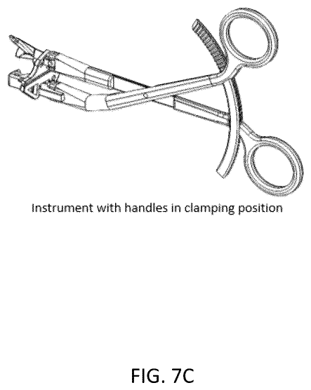

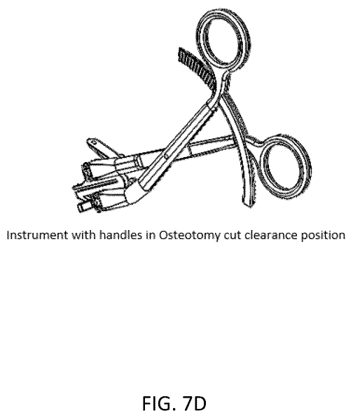

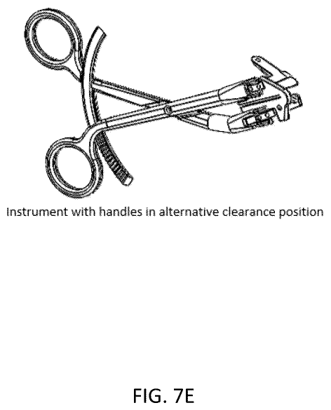

In FIG. 7A, a perspective view of a ROSE Osteotomy Guide Clamp with handles open, prior to clamping generally represented by reference numeral 70 and utilized in a ROSE bunionectomy procedure is shown, according to the present disclosure. The ROSE Guide Clamp 70 may comprise a plantar pad 71, a plantar pad clearance slot 72, an osteotomy jig 73, a handle rotation features with detent 74, a locking ratchet feature 75, a plantar handle 76, a dorsal handle 77, and a handle pivot 78.

In FIG. 7B, a cross-sectional view of a spring loaded detent used in a ROSE Osteotomy Guide Clamp generally represented by reference numeral 70 and utilized in a ROSE bunionectomy procedure is shown, according to the present disclosure. The ROSE Guide Clamp 70 may comprise a spring loaded pin (detent) 79.

In FIG. 7C, a ROSE Osteotomy Guide Clamp with handles in a clamping position generally represented by reference numeral 70 and utilized in a ROSE bunionectomy procedure is shown, according to the present disclosure. The ROSE Guide Clamp 70 may comprise a plantar pad 71, a plantar pad clearance slot 72, an osteotomy jig 73, a handle rotation features with detent 74, a locking ratchet feature 75, a plantar handle 76, a dorsal handle 77, and a handle pivot 78.

In FIG. 7D, a ROSE Osteotomy Guide Clamp with handles in an Osteotomy cut clearance position generally represented by reference numeral 70 and utilized in a ROSE bunionectomy procedure is shown, according to the present disclosure. The ROSE Guide Clamp 70 may comprise a plantar pad 71, a plantar pad clearance slot 72, an osteotomy jig 73, a handle rotation features with detent 74, a locking ratchet feature 75, a plantar handle 76, a dorsal handle 77, and a handle pivot 78.

In FIG. 7E, a ROSE Osteotomy Guide Clamp with handles in an alternative clearance position generally represented by reference numeral 70 and utilized in a ROSE bunionectomy procedure is shown, according to the present disclosure. The ROSE Guide Clamp 70 may comprise a plantar pad 71, a plantar pad clearance slot 72, an osteotomy jig 73, a handle rotation features with detent 74, a locking ratchet feature 75, a plantar handle 76, a dorsal handle 77, and a handle pivot 78.

### Securing the ROSE Osteotomy Guide Clamp on the Exposed First Metatarsal

The ROSE Osteotomy Guide Clamp (ROSE Guide Clamp) handles are in the clamping position when the rotational folding handles are essentially perpendicular to the near face of the ROSE jig portion of the instrument. Spring loaded detents provide tactile feedback to the surgeon of the correct handle position and prevent inadvertent dislodging from this orientation. In one embodiment of the ROSE Guide Clamp of the present disclosure, these detents may be spring loaded.

The upper and lower portions of the ROSE Guide Clamp can be spaced open by expanding the handles, then placed over and around the exposed first metatarsal, with the upper portion of the clamp intended to contact the dorsal portion of the exposed first metatarsal and the lower portion of the clamp intended to contact the plantar portion.

Similar to the stand-alone ROSE Osteotomy Jig described herein, the ROSE Guide Clamp is first positioned on the exposed first metatarsal using surgeon markings and/or anatomical features on the first metatarsal bone. This may include a mark applied by the surgeon to the mid-line dorsal surface of the exposed metatarsal bone, prior to ROSE Guide Clamp placement, which is then aligned with markings on the instrument. Additionally, the clamp's integrated ROSE jig may comprise organically designed bosses and surfaces to contact and mate with typical bone surface geometry.

Once properly placed, the ROSE jig can be fixated to the metatarsal bone in two medial locations by drilling olive wires, or similar, through each of two wire location holes in the near face of the jig. These fixation wires enter the near face of the clamp, pass through the jig, and enter the medial face of the metatarsal. Countersinks on the near face of the jig, at the wire location holes, are intended to mate with olive-style orthopedic wires, which may be the preferred fixation wire type. The jig feature is then mounted flush against the medial side of the exposed first metatarsal.

Once the upper portion of the instrument is properly positioned and secured with wires, the device is securely clamped around the metatarsal by rotating the handles to the clamping position. The clamp is secured by squeezing the handles until the lower portion of the clamp passes along the plantar pad clearance slot in the ROSE jig portion of the instrument and engages the plantar surface of the metatarsal. The dorsal pad feature of the upper portion of the clamp securely engages the dorsal surface of the metatarsal, and the plantar pad feature of the lower portion of the clamp (plantar pad) securely engages the plantar surface of the metatarsal. The pads are designed to mate with typical anatomical contours of the metatarsal, allowing for the proper placement and firm retention of the device. Additionally, the pads sizes are as small as possible in order to help reduce the size of the incision.

Ratcheting locking features on the instrument handles retain the secure clamping.

Once the device is securely clamped on the metatarsal, the rotational guide K-wire (e.g., olive wire, K-wire, and/or guidewire, etc.) is inserted. This guide wire is inserted using the rotational wire guide through a hole on the dorsal portion of the ROSE jig section of the clamp. In one embodiment of the Rose Guide Clamp of the present disclosure, the rotation wire guide may include a threaded portion to accept a standard orthopedic threaded drill guide (a.k.a., tower). The wire is drilled from the dorsal surface of the first metatarsal, approximately ⅓ into the dorsal cortex. The wire should not fully penetrate through the metatarsal, with an insertion depth that will not interfere with the osteotomy cut. In one embodiment of the ROSE instruments of the present disclosure, this rotational K-wire may be color coded or etched to provide a visual indication to the surgeon of this recommended K-wire insertion depth.

### Preparing for the Osteotomy Cut

The ROSE jig portion of the ROSE Guide Clamp provides an intuitive sagittal saw blade guide for the osteotomy cut, and ensures the cut is perpendicular to the axis of the rotational K-wire. Maintaining this perpendicular relationship maximizes the weight bearing surface of the corrected bunion without changing the overall length of the metatarsal. It ensures that the compression screw fixation is perpendicular to the osteotomy in order to apply maximum compression against the osteotomy.

The ROSE Guide Clamp may comprise a rotationally folding hinge design that connects the lower handle to the dorsal pad portion of the upper clamp (i.e., upper hinge). A similar hinge design connects the upper handle to the plantar pad portion of the lower clamp (i.e., lower hinge).

To maximize clamp force, the axis of the upper hinge is essentially collinear to the axis of the lower hinge.

The ROSE Guide Clamp handles can be rotated in unison with the hinge axis as the center of rotation. Detents within the hinge assembly, with one device embodiment incorporating spring loaded detents, allow the device handles to be retained in set angular positions including but not limited to a distal position, clamping position, and/or proximal position.

To clamp the device, the handles are rotated to a position essentially perpendicular to the lateral plane of the metatarsal. When the device handles are squeezed together, the hinges transmit the applied force to clamp the dorsal and plantar pads securely against the metatarsal. The pads are designed to conform to specific anatomical contours of the metatarsal, further reinforcing the proper placement and retention of the device.

When in the clamping position, the handles prevent full visual exposure of the osteotomy guide slot for the surgeon and physical interference with the sagittal saw. To remedy this, the handles can be rotationally folded towards the distal end of the metatarsal and retained in the distal detent position by spring loaded detents.

Once the instrument is correctly placed on the exposed first metatarsal, medially fixated with olive wires, and securely clamped, the rotational K-wire is drilled ⅓ into the dorsal cortex of the metatarsal. The wire should not fully penetrate through the metatarsal, with an insertion depth that does not interfere with the osteotomy cut. Further, the surgeon prepares for the osteotomy cut. With the instrument handles in the clamping position (e.g., laterally perpendicular), the surgeon slightly reduces the clamping force to allow space for bone separation by releasing the handle ratchets, readjusting the handles, then positioning the rotationally folding handles in the distal position. Detents provide tactile feedback to the surgeon of the current orientation and prevent inadvertent dislodging from this position. In one embodiment of the ROSE Guide Clamp, these detents may be spring loaded.

### Executing the Osteotomy Cut

Once the instrument is correctly placed on the exposed first metatarsal, medially fixated with olive wires, with slightly relaxed clamping, with folding handles in the distal position, and the rotational K-wire partially drilled to ensure sufficient clearance for the osteotomy, approximately ⅓ into the dorsal cortex, the surgeon can execute the osteotomy cut.

The ROSE jig portion of the ROSE Guide Clamp instrument may comprise an intuitive guide for the proper blade insertion at the distal dorsal end of the cut guide, controlled direction of the cut from the distal dorsal to plantar proximal end of the cut guide, and removal of the sagittal saw blade at the plantar proximal end of the cut guide. This allows for the proper execution of the ROSE osteotomy cut, with respect to the anatomy of the first (and/or fifth) metatarsal as well as the correction axis of rotation, controlled later in the procedure by the placement of the drilled rotational wire guide.

Once the osteotomy cut has been successfully executed, the dorsal and plantar fragments are securely clamped in preparation for the full rotational K-wire (e.g., olive wire, K-wire, and/or guidewire, etc.) insertion.

Securing Fragments for Correction Preparation without Device Removal

A novel benefit of the ROSE Guide Clamp, with the integrated ROSE jig, is the option to utilize the rotationally folding handles to secure the bone fragments without the need to remove the instrument. With the osteotomy guide complete, the distally folded handles can be returned to the clamping position.

The device handles can be rotated in unison with the hinge axis as the center of rotation. Detents within the hinge assembly, wherein one device embodiment may comprise spring loaded detents, allow the device handles to be retained in set angular positions including but not limited to a distal position, clamping position, and/or proximal position.

To secure the fragments, the handles are set in a position essentially perpendicular to the lateral plane of the metatarsal (e.g., ‘clamping’ position). Detents provide tactile feedback to the surgeon of the current orientation and prevent inadvertent dislodging from this position. In one embodiment of the instrument, these detents may be spring loaded.

When the device handles are squeezed together, the hinges transmit the applied force to clamp the dorsal and plantar pads securely against each metatarsal fragment. The pads are designed to conform to specific anatomical contours of the metatarsal, helping to guide the fragments to the proper relative position. The clamp pads are placed perpendicular to the osteotomy cut, and therefore, secure the osteotomy most effectively.

### Full Insertion of Rotational Wire

With the metatarsal dorsal and plantar fragments securely clamped, the rotational wire can now be fully inserted through both the dorsal and plantar fragments. The rotational wire is fully drilled from the dorsal surface of the first metatarsal dorsal fragment through the plantar surface of the plantar fragment, perpendicularly crossing the osteotomy cut surfaces. In one embodiment of the ROSE instrument set, the rotational K-wire is color coded or etched to provide a visual indication to the surgeon of this fully inserted K-wire (e.g., olive wire, K-wire, and/or guidewire, etc.) insertion depth.

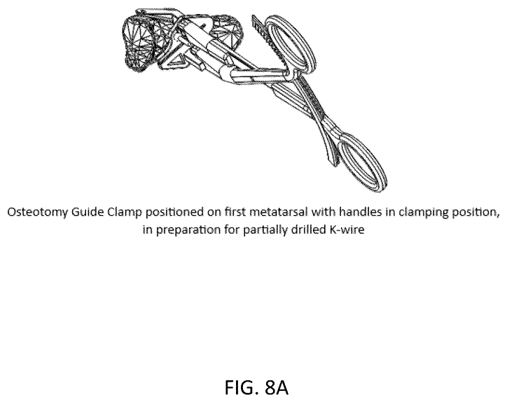

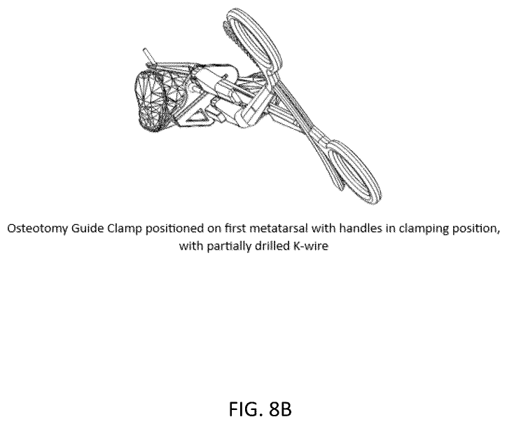

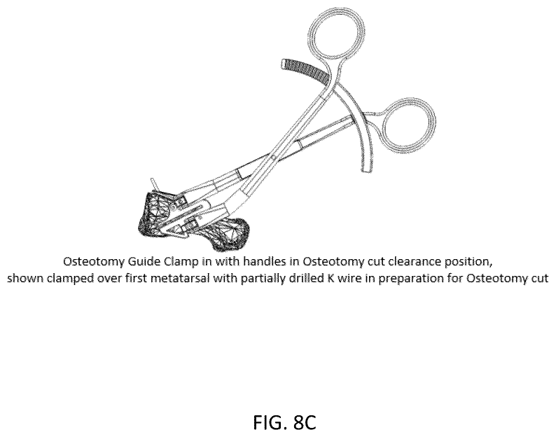

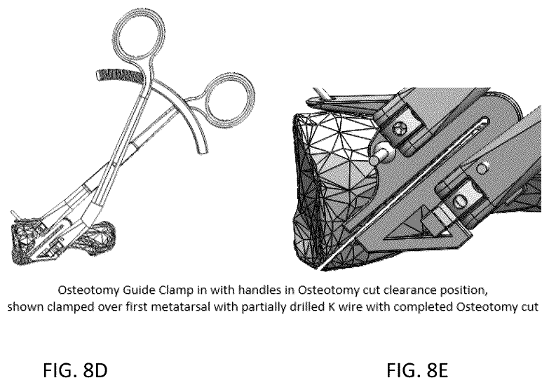

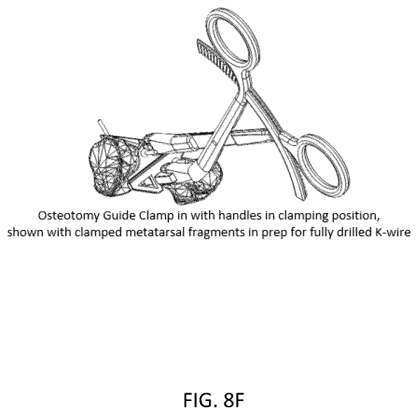

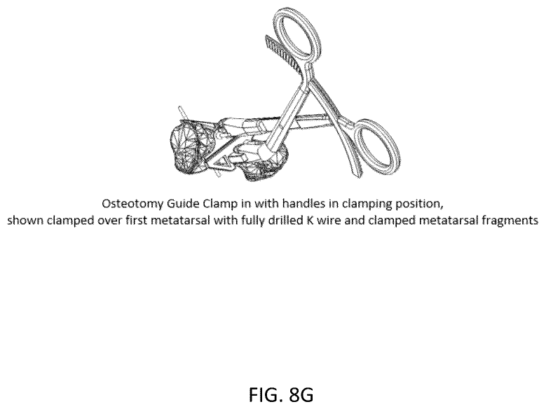

In FIG. 8A, a ROSE Osteotomy Guide Clamp positioned on the first metatarsal with handles in a clamping position, in preparation for insertion of a partially drilled K-wire generally represented by reference numeral 80 and utilized in a ROSE bunionectomy procedure is shown, according to the present disclosure. The ROSE Guide Clamp 80 may comprise a plantar pad 81, a plantar pad clearance slot 82, an osteotomy jig 83, a handle rotation features with detent 84, a locking ratchet feature 85, a plantar handle 86, a dorsal handle 87, and a handle pivot 88.

In FIG. 8B, a ROSE Osteotomy Guide Clamp positioned on the first metatarsal with handles in a clamping position, with insertion of a partially drilled K-wire generally represented by reference numeral 80 and utilized in a ROSE bunionectomy procedure is shown, according to the present disclosure. The ROSE Guide Clamp 80 may comprise a plantar pad 81, a plantar pad clearance slot 82, an osteotomy jig 83, a handle rotation features with detent 84, a locking ratchet feature 85, a plantar handle 86, a dorsal handle 87, a handle pivot 88, and a K-wire 800.

In FIG. 8C, a ROSE Osteotomy Guide Clamp with handles in an Osteotomy cut clearance position, clamped over the first metatarsal with insertion of a partially drilled K-wire in preparation for an Osteotomy cut generally represented by reference numeral 80 and utilized in a ROSE bunionectomy procedure is shown, according to the present disclosure. The ROSE Guide Clamp 80 may comprise a plantar pad 81, a plantar pad clearance slot 82, an osteotomy jig 83, a handle rotation features with detent 84, a locking ratchet feature 85, a plantar handle 86, a dorsal handle 87, a handle pivot 88, and a K-wire 800.

In FIGS. 8D and 8E, a full view and close up view of a ROSE Osteotomy Guide Clamp with handles in an Osteotomy cut clearance position, clamped over the first metatarsal with insertion of a partially drilled K-wire with a completed Osteotomy cut generally represented by reference numeral 80 and utilized in a ROSE bunionectomy procedure are shown, according to the present disclosure. The ROSE Guide Clamp 80 may comprise a plantar pad 81, a plantar pad clearance slot 82, an osteotomy jig 83, a handle rotation features with detent 84, a locking ratchet feature 85, a plantar handle 86, a dorsal handle 87, a handle pivot 88, a K-wire 800, an osteotomy cut 801, a dorsal metatarsal fragment 802, and a plantar metatarsal fragment 803.

In FIG. 8F, a ROSE Osteotomy Guide Clamp with handles in a clamping position, with clamped metatarsal fragments in preparation for insertion of a fully drilled K-wire generally represented by reference numeral 80 and utilized in a ROSE bunionectomy procedure is shown, according to the present disclosure. The ROSE Guide Clamp 80 may comprise a plantar pad 81, a plantar pad clearance slot 82, an osteotomy jig 83, a handle rotation features with detent 84, a locking ratchet feature 85, a plantar handle 86, a dorsal handle 87, a handle pivot 88, a K-wire 800, an osteotomy cut 801, a dorsal metatarsal fragment 802, and a plantar metatarsal fragment 803.

In FIG. 8G, a ROSE Osteotomy Guide Clamp with handles in a clamping position, clamped over the first metatarsal with a fully drilled K-wire and clamped metatarsal fragments generally represented by reference numeral 80 and utilized in a ROSE bunionectomy procedure is shown, according to the present disclosure. The ROSE Guide Clamp 80 may comprise a plantar pad 81, a plantar pad clearance slot 82, an osteotomy jig 83, a handle rotation features with detent 84, a locking ratchet feature 85, a plantar handle 86, a dorsal handle 87, a handle pivot 88, a K-wire 800, an osteotomy cut 801, a dorsal metatarsal fragment 802, and a plantar metatarsal fragment 803.

### ROSE Intermetatarsal (IM) Correction Clamp

The ROSE Bunion Correction system and method may comprise a ROSE Intermetatarsal (IM) Correction Clamp (hereinafter “ROSE Correction Clamp”). A key benefit of the ROSE procedure is the perpendicular relationship between the correction (i.e., reductions) axis and the osteotomy cut plane. With this relationship, the ROSE bunion correction maximizes the fragment bearing surfaces without a change in the overall length of the metatarsal.

The ROSE Bunion Correction system and method may comprise a unique IM Correction Clamp (i.e., ROSE Correction Clamp) that can rotate the first metatarsal bone around an axis pin to correct the first intermetatarsal angle without compromising this perpendicular relationship.

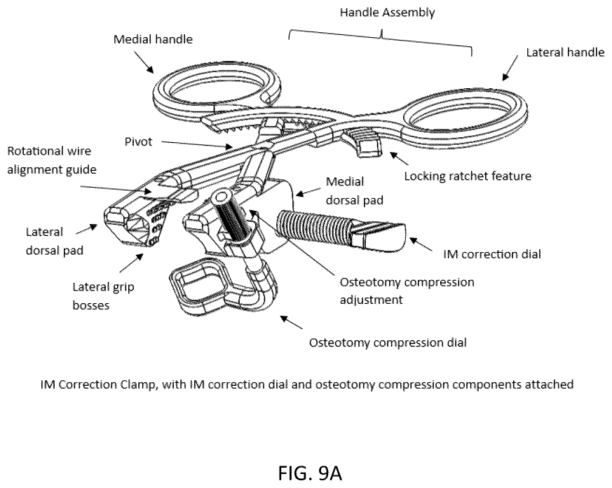

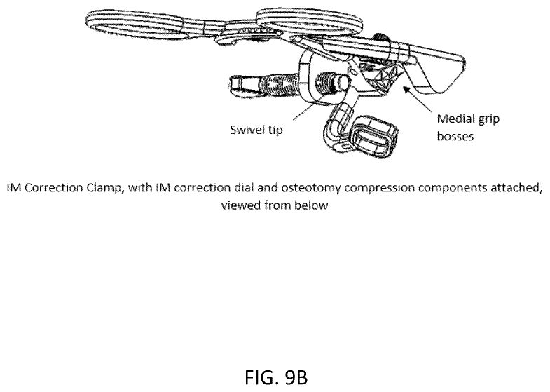

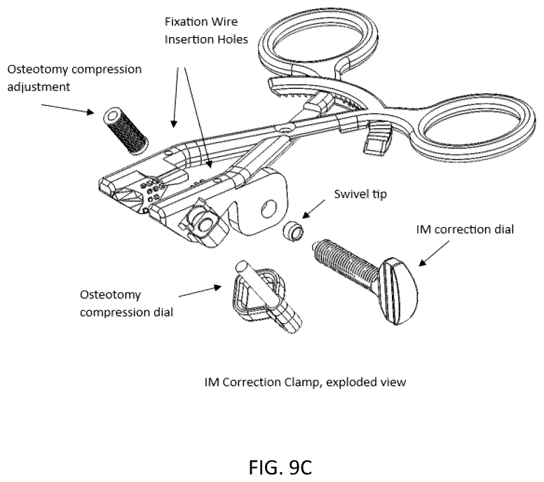

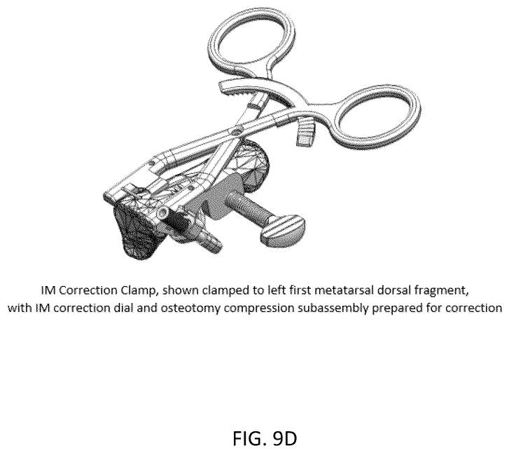

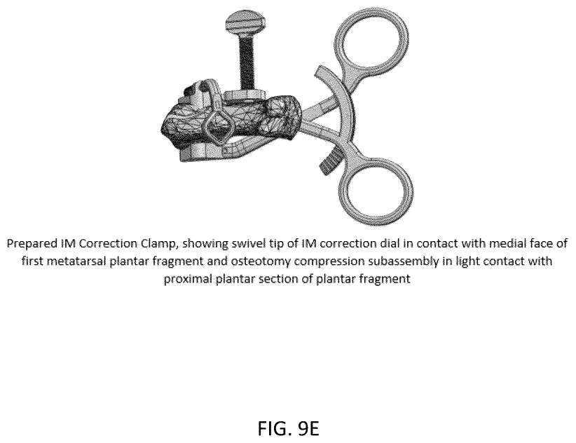

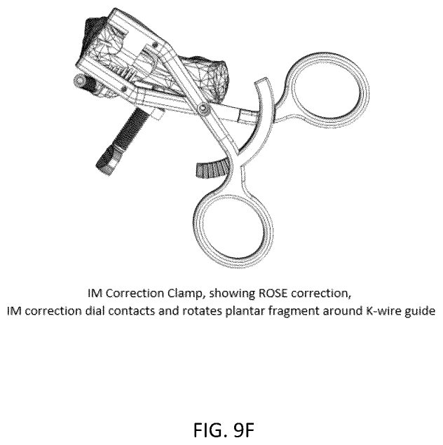

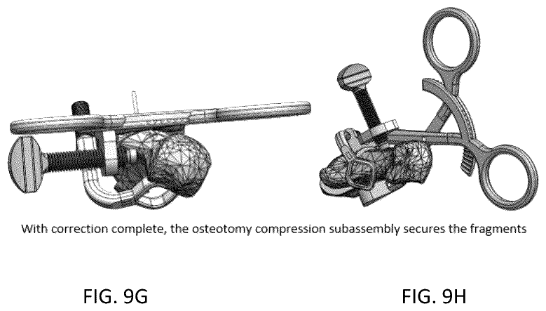

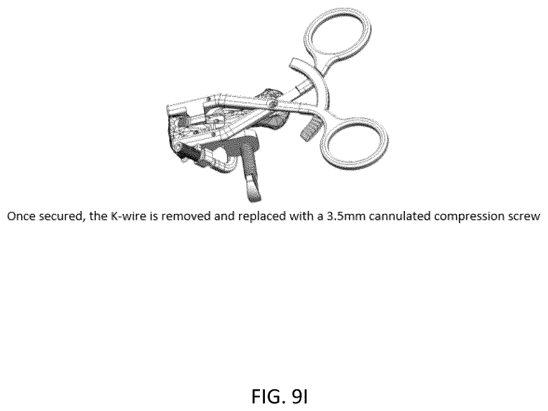

In FIG. 9A, a ROSE Intermetatarsal (IM) Correction Clamp with an IM correction dial and attached osteotomy compression components generally represented by reference numeral 90 and utilized in a ROSE bunionectomy procedure is shown, according to the present disclosure. The ROSE Correction Clamp 90 may comprise a lateral grip bosses 91, a lateral dorsal pad 92, a rotational wire alignment guide 93, a pivot 94, a medial handle 95, a lateral handle 96, a locking ratchet feature 97, a medial dorsal pad 98, an IM correction dial 99, an osteotomy compression adjustment 900, and an osteotomy compression dial 901.

In FIG. 9B, a below view of a ROSE Intermetatarsal (IM) Correction Clamp with an IM correction dial and attached osteotomy compression components generally represented by reference numeral 90 and utilized in a ROSE bunionectomy procedure is shown, according to the present disclosure. The ROSE Correction Clamp 90 may comprise a lateral grip bosses 91, a lateral dorsal pad 92, a rotational wire alignment guide 93, a pivot 94, a medial handle 95, a lateral handle 96, a locking ratchet feature 97, a medial dorsal pad 98, an IM correction dial 99, an osteotomy compression adjustment 900, an osteotomy compression dial 901, a swivel tip 902, and a medial grip bosses 903.

In FIG. 9C, an exploded view of a ROSE Intermetatarsal (IM) Correction Clamp with an IM correction dial and attached osteotomy compression components generally represented by reference numeral 90 and utilized in a ROSE bunionectomy procedure is shown, according to the present disclosure. The ROSE Correction Clamp 90 may comprise a lateral grip bosses 91, a lateral dorsal pad 92, a rotational wire alignment guide 93, a pivot 94, a medial handle 95, a lateral handle 96, a locking ratchet feature 97, a medial dorsal pad 98, an IM correction dial 99, an osteotomy compression adjustment 900, an osteotomy compression dial 901, a swivel tip 902, a medial grip bosses 903, and fixation wire insertion holes 904.

### Preparing for IM Correction

To ensure the angular correction occurs around the optimal axis of rotation, the novel ROSE Correction Clamp may comprise an integrated rotational wire alignment guide. This guide interacts with the previously inserted rotational K-wire. This rotational wire acts as an axis pin for the relative correction motion (e.g., reduction) of the dorsal and plantar metatarsal fragments.

The ROSE Correction Clamp is positioned over and securely clamps to the proximal portion of the dorsal fragment. The medial and lateral dorsal clamp pads are designed to mate with typical anatomical contours of the metatarsal, allowing for the proper placement and firm retention of the device. These dorsal pads have a minimal geometry to contact the surface of the dorsal metatarsal fragment correctly in this relatively small available metatarsal surface space and incision clearance. Grip bosses help ensure proper placement and retention.

Additionally, the clamp can be further fixated to the metatarsal bone by drilling olive wires, and/or similar, into each of two fixation wire insertion holes.

Ratcheting features on the instrument handles retain the secure clamping.

### Executing the IM Correction

Correction of the intermetatarsal angle, also known as the reduction, occurs by manually adjusting the depth of the swivel tipped IM correction dial. The IM correction dial is a removable component of the instrument, and the swivel tip can be a replaceable item. This allows for ease of cleaning and sterilization. The IM correction dial can be readily prepared by the surgical support team prior to use by pressing the swivel tip over the ball end feature of the IM correction dial and threading the prepared IM correction dial through the mating feature on the medial side of the instrument. As a removable and/or selectable component, the design offers the surgeon flexibility in terms of type and size of swivel tip, screw length, degree of correction, etc.

In an alternative embodiment of the ROSE Correction Clamp, the IM correction dial swivel tip can have a wider base to allow the correction from outside of the incision. The wider base initiates a relative fragment rotation about the K-wire by applying a moment through the skin with minimized surface pressure. Note: the wider base would be attached to the IM correction dial after the IM correction dial is assembled through the clamp.

The IM correction dial assembly (IM correction dial with swivel tip) is designed to work with either the left-handed or right-handed version of the instrument.

The design of the ROSE Correction Clamp allows easy viewing of the relative angular motion while the IM correction dial is adjusted. Alternative embodiments of the design may comprise a visual scale integrated in the instrument, or color markings on the correction dial threads, to track and/or record the degree of correction during the adjustment.

### Securing the IM Correction

A secondary screw adjustment, the osteotomy compression subassembly, holds the fragment correction in place. This two-piece subassembly is removable from the instrument, consisting of an internally threaded Osteotomy compression adjustment component and an externally threaded Osteotomy compression dial with pin/screw clearance. This removable, two-piece design allows for ease of cleaning and sterilization. The IM correction dial can be readily prepared by the surgical support team prior to use. The Osteotomy compression adjustment component is placed within the receptacle feature on the medial portion of the instrument, the hook portion is inserted through the clearance hole on the near side of the medial portion of the instrument, and the Osteotomy compression adjustment is rotated to engage and adjust the distance of the Osteotomy compression dial feature from the corrected metatarsal fragment. As a removable and selectable component, the osteotomy compression subassembly design offers the surgeon flexibility in terms of type of hook design, degree of correction, etc.

The osteotomy compression subassembly is designed to work with either the left-handed or right-handed version of the instrument.

To securely retain the correction, the Osteotomy compression adjustment is adjusted by the surgeon until the Osteotomy compression dial firmly contacts the plantar fragment in the proximal area. The ROSE Correction Clamp may comprise a hook, which features a clearance area intended to mate under and around the exit portion of the previously installed rotational K-wire. Additionally, the geometry of the hook is designed to mate with typical anatomical contours of the metatarsal plantar fragment, allowing for the proper placement and firm retention of the correction.

The metatarsal fragments are corrected and securely retained in this corrected position. This novel ROSE Correction Clamp, with its dorsal rotational wire alignment feature and plantar wire clearance feature, allows for the removal of the rotational K-wire and insertion of an orthopedic implant, typically a 3.5 mm, or other size compression screw, without the need for removal of the ROSE Correction Clamp instrument. With the instrument retained, these fragments will not shift out of position during compression screw fixation and retain the ROSE Bunion Correction benefits described herein.

Once the compression screw implant is properly placed, the IM correction dial and osteotomy compression components are loosened, the locking handle ratchets are released, and the ROSE Correction Clamp is removed.

In FIG. 9D, a ROSE Intermetatarsal (IM) Correction Clamp, which is clamped to the left first metatarsal dorsal fragment, with an IM correction dial and an osteotomy compression subassembly prepared for correction generally represented by reference numeral 90 and utilized in a ROSE bunionectomy procedure is shown, according to the present disclosure. The ROSE Correction Clamp 90 may comprise a lateral grip bosses 91, a lateral dorsal pad 92, a rotational wire alignment guide 93, a pivot 94, a medial handle 95, a lateral handle 96, a locking ratchet feature 97, a medial dorsal pad 98, an IM correction dial 99, an osteotomy compression adjustment 900, an osteotomy compression dial 901, a swivel tip 902, a medial grip bosses 903, fixation wire insertion holes 904, a dorsal fragment 905, and a plantar fragment 906.

In FIG. 9E, a ROSE Intermetatarsal (IM) Correction Clamp, which is prepared with a swivel tip of an IM correction dial in contact with the medial face of the first metatarsal plantar fragment and an osteotomy compression subassembly, in light contact with a proximal plantar section of the plantar fragment generally represented by reference numeral 90 and utilized in a ROSE bunionectomy procedure is shown, according to the present disclosure. The ROSE Correction Clamp 90 may comprise a lateral grip bosses 91, a lateral dorsal pad 92, a rotational wire alignment guide 93, a pivot 94, a medial handle 95, a lateral handle 96, a locking ratchet feature 97, a medial dorsal pad 98, an IM correction dial 99, an osteotomy compression adjustment 900, an osteotomy compression dial 901, a swivel tip 902, a medial grip bosses 903, fixation wire insertion holes 904, a dorsal fragment 905, and a plantar fragment 906.

In FIG. 9F, a ROSE Intermetatarsal (IM) Correction Clamp with a completed ROSE correction, whereby an IM correction dial contacts and rotates a plantar fragment around a K-wire guide generally represented by reference numeral 90 and utilized in a ROSE bunionectomy procedure is shown, according to the present disclosure. The ROSE Correction Clamp 90 may comprise a lateral grip bosses 91, a lateral dorsal pad 92, a rotational wire alignment guide 93, a pivot 94, a medial handle 95, a lateral handle 96, a locking ratchet feature 97, a medial dorsal pad 98, an IM correction dial 99, an osteotomy compression adjustment 900, an osteotomy compression dial 901, a swivel tip 902, a medial grip bosses 903, fixation wire insertion holes 904, a dorsal fragment 905, and a plantar fragment 906.

In FIGS. 9G and 9H, a below view and side view of a ROSE Intermetatarsal (IM) Correction Clamp with a completed ROSE correction, whereby an osteotomy compression subassembly secures the dorsal and plantar fragments generally represented by reference numeral 90 and utilized in a ROSE bunionectomy procedure are shown, according to the present disclosure. The ROSE Correction Clamp 90 may comprise a lateral grip bosses 91, a lateral dorsal pad 92, a rotational wire alignment guide 93, a pivot 94, a medial handle 95, a lateral handle 96, a locking ratchet feature 97, a medial dorsal pad 98, an IM correction dial 99, an osteotomy compression adjustment 900, an osteotomy compression dial 901, a swivel tip 902, a medial grip bosses 903, fixation wire insertion holes 904, a dorsal fragment 905, a plantar fragment 906, and a K-wire 907.

In FIG. 9I, a ROSE Intermetatarsal (IM) Correction Clamp with a completed ROSE correction and a secured osteotomy compression subassembly, wherein a K-wire is removed and replaced with a 3.5 mm cannulated compression screw generally represented by reference numeral 90 and utilized in a ROSE bunionectomy procedure is shown, according to the present disclosure. The ROSE Correction Clamp 90 may comprise a lateral grip bosses 91, a lateral dorsal pad 92, a rotational wire alignment guide 93, a pivot 94, a medial handle 95, a lateral handle 96, a locking ratchet feature 97, a medial dorsal pad 98, an IM correction dial 99, an osteotomy compression adjustment 900, an osteotomy compression dial 901, a swivel tip 902, a medial grip bosses 903, fixation wire insertion holes 904, a dorsal fragment 905, a plantar fragment 906, and a compression screw 908 (e.g., cannulated/non-cannulated, and 3.5 mm or other size).

### ROSE Fixation Clamp

Once the 3.5 mm, or other size compression screw is fully secured through the corrected dorsal and plantar metatarsal fragments, the bone fragments are fully fixated using either a dorsal or dorsal plantar (DP) ROSE bone plate. Please see the following sections for more details on these plate designs described herein.

The ROSE Bunion Correction system and method may comprise a ROSE Fixation Clamp. The ROSE Fixation Clamp is uniquely designed to aid the surgeon in proper bone plate placement and retention during the fixation procedure step, regardless of IM correction angle. It is suitable for use with either the dorsal or DP ROSE bone plate design option.

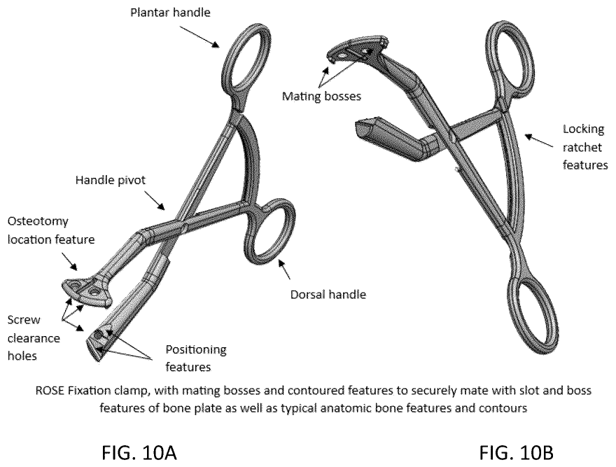

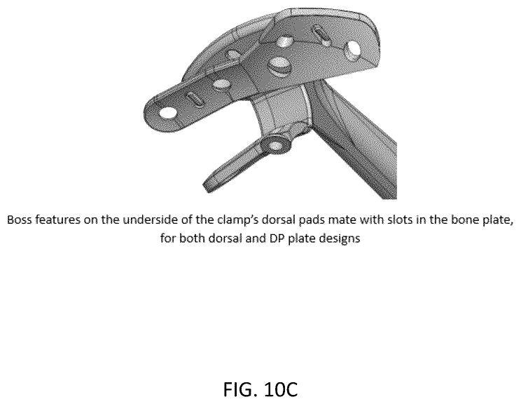

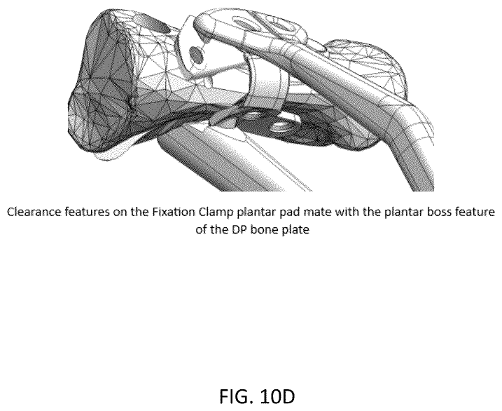

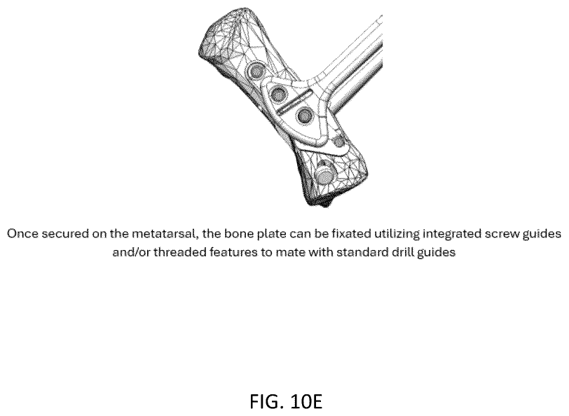

In FIGS. 10A and 10B, a top view and bottom view of a ROSE Fixation Clamp with mating bosses and contoured features to securely mate with slot and boss features of bone plate, as well as typical anatomic bone features and contours generally represented by reference numeral 100 and utilized in a ROSE bunionectomy procedure are shown, according to the present disclosure. The ROSE Fixation Clamp 100 may comprise screw clearance holes 101, an osteotomy location feature 102, a handle pivot 103, a plantar handle 104, a dorsal handle 105, positioning features 106, mating bosses 107, and locking ratchet features 108.

### Preparing for Fixation

Typically, the dorsal metatarsal portion is cleaned to remove any overhang resulting from the IM correction. The medial dorsal surface is ground smooth at the osteotomy interface with a burr drill before the next instrument is used.

The upper portion on the ROSE Fixation Clamp may comprise a dorsal pad with alignment marking and/or through slots for proper positioning with respect to the osteotomy cut, through holes to guide two of the plate screws, and two plate fixturing bosses. These bosses are intended to temporarily retain the plate during placement of the clamp over the corrected metatarsal and during initial screw fixation.

The lower portion of the ROSE Fixation Clamp may comprise a plantar pad designed to mate with typical anatomical contours of the metatarsal bone. Additionally, the Fixation Clamp may comprise a plantar pad with unique features to accept and retain the plantar portion of the DP bone plate.

In one embodiment of the ROSE Fixation Clamp, the screw clearance holes may comprise a threaded portion to accept a standard orthopedic threaded drill guide (a.k.a., tower) to ensure proper directional placement of the screws. Proper directional placement ensures the screws are sufficiently spaced away from the osteotomy surface edge (e.g., at the outer bone surface interface), do not interfere with other fixation screws, and are sufficiently spaced from other screws to alleviate any stress risers.

Fixation with the Dorsal Bone Plate

When using the dorsal bone plate design, the plate is retained with two plate fixturing bosses on the ROSE Fixation Clamp's dorsal pad and held securely on the corrected metatarsal between the dorsal and plantar pads. Further, the Fixation Clamp is securely clamped by squeezing the handles. Ratcheting features on the instrument handles retain the secure clamping.

At least two 2.4 mm, or other sized locking or non-locking screws are inserted through the screw clearance holes of the ROSE Fixation Clamp. These may be inserted with the aid of a threaded drill guide. Once these two locking or non-locking screws are securely fastened, the Fixation Clamp is removed from the metatarsal by releasing the ratcheting features on the instrument handles. The remaining locking or non-locking screws are inserted.

Fixation with the Dorsal Plantar (DP) Bone Plate

When using the Dorsal Plantar (hereinafter “DP”) bone plate design, additional procedural steps are required to ensure the plate provides additional clamping (i.e., compression effect) with a non-locking bone screw (a.k.a., clamping screw) that passes through the dorsal portion of the DP bone plate and securely threads (e.g., fastens) into an axially aligned internally threaded boss on the plantar portion of the plate.

To ensure this proper axial alignment, a positioning K-wire is drilled from the dorsal portion of the ROSE Fixation Clamp, through the metatarsal, exiting through the boss portion of the plantar pad. In one embodiment of the DP bone plate design, the Fixation Clamp's dorsal screw clearance holes include a threaded feature to enable the use of a threaded drill guide. The K-wire exits cleanly through the tapped boss on the DP bone plate. If needed, the Fixation Clamp may be readjusted, and the wire redrilled. Once the alignment is verified, the threaded K-wire drill guide is removed and a 2.4 mm, or other size cannulated clamping bone screw is inserted over the guide wire.

A 2.4 mm, or other size locking or non-locking screw is inserted through the distal screw clearance hole on the ROSE Fixation Clamp's dorsal pad. This may be inserted with the aid of a threaded drill guide. Once this locking or non-locking screw is securely fastened, the Fixation Clamp is removed from the metatarsal by releasing the ratcheting features on the instrument handles. The remaining locking or non-locking screws are inserted.

In FIG. 10C, a ROSE Fixation Clamp with boss features on the underside of the Fixation Clamp's dorsal pads mating with slots in the bone plate, for both dorsal and DP bone plate designs generally represented by reference numeral 100 and utilized in a ROSE bunionectomy procedure is shown, according to the present disclosure. The ROSE Fixation Clamp 100 may comprise screw clearance holes 101, an osteotomy location feature 102, mating bosses 107, and a dorsal plantar (DP) bone plate 109 (or dorsal bone plate).

In FIG. 10D, a ROSE Fixation Clamp with clearance features on the Fixation Clamp's plantar pad, mating with the plantar boss feature of the DP bone plate generally represented by reference numeral 100 and utilized in a ROSE bunionectomy procedure is shown, according to the present disclosure. The ROSE Fixation Clamp 100 may comprise screw clearance holes 101, an osteotomy location feature 102, positioning features 106, mating bosses 107, and a dorsal plantar (DP) bone plate 109 (or dorsal bone plate).

In FIG. 10E, a ROSE Fixation Clamp is secured on a metatarsal, wherein the bone plate is fixated utilizing integrated screw guides and/or threaded features to mate with standard drill guides generally represented by reference numeral 100 and utilized in a ROSE bunionectomy procedure is shown, according to the present disclosure. The ROSE Fixation Clamp 100 may comprise screw clearance holes 101, an osteotomy location feature 102, positioning features 106, mating bosses 107, a dorsal plantar (DP) bone plate 109 (or dorsal bone plate), and screw guides/threaded features 1000.

### Reverdin Jig

The ROSE Bunion Correction system and method may comprise a Reverdin Jig. The Reverdin Jig is designed to readily determine proper positioning of the Reverdin correction (e.g., wedge) cut and correct articular deviation set angle. However, it can be used to further reduce the IM angle as defined below at the metatarsal head. Further, it can be used together with the ROSE bunionectomy procedure and/or as a standalone correction protocol.

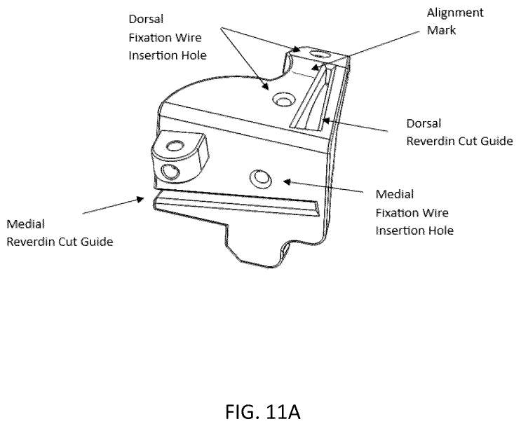

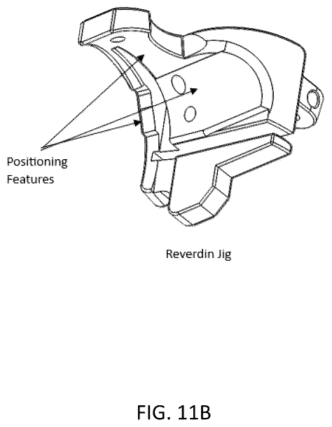

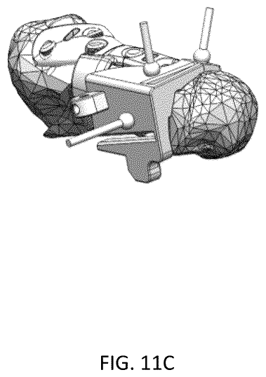

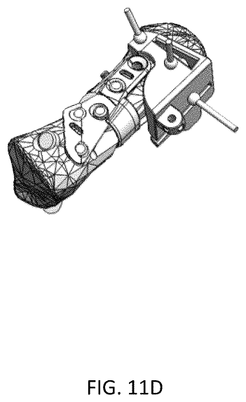

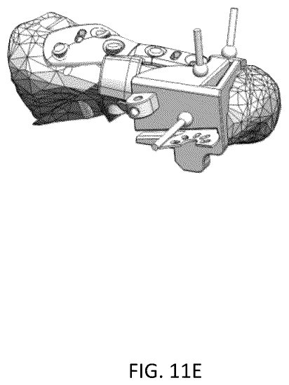

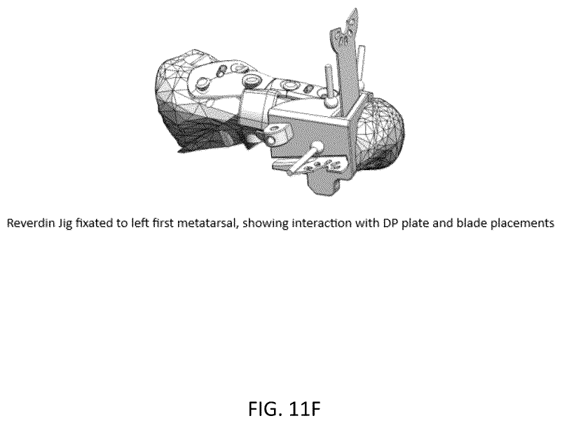

In FIG. 11A, a top view of a Reverdin Jig generally represented by reference numeral 110 and utilized in a ROSE bunionectomy procedure and/or Reverdin correction procedure is shown, according to the present disclosure. The Reverdin Jig 110 may comprise a medial Reverdin cut guide 111, a dorsal fixation wire insertion hole 112, an alignment mark 113, a dorsal Reverdin cut guide 114, and a medial fixation wire insertion hole 115.

In FIG. 11B, a bottom view of a Reverdin Jig generally represented by reference numeral 110 and utilized in a ROSE bunionectomy procedure and/or Reverdin correction procedure is shown, according to the present disclosure. The Reverdin Jig 110 may comprise a medial Reverdin cut guide 111, a dorsal fixation wire insertion hole 112, an alignment mark 113, a dorsal Reverdin cut guide 114, a medial fixation wire insertion hole 115, and positioning features 116.

The Reverdin Jig may comprise positioning features geometrically designed to mate with typical anatomic bone contours, fixation features, a slot features (i.e., one dorsal, one medial) to properly guide a sagittal saw cut fully through the metatarsal bone from a medial insertion to a lateral plantar exit, and a sagittal saw blade cut partially through the metatarsal bone from a dorsal insertion, while not procuring through the plantar cortex.

### Placement and Fixturing of the Reverdin Jig

To utilize this instrument, the Reverdin Jig is first positioned using surgeon markings and/or anatomical features on the first metatarsal bone. This may include a mark applied by the surgeon to the mid-line dorsal surface of the exposed metatarsal bone prior to Reverdin Jig placement, which is then aligned with markings on the jig. Additionally, the Reverdin Jig may comprise organically designed bosses and surfaces to contact and mate with typical bone surface geometry.

In FIG. 11C, a perspective view of a Reverdin Jig fixated to the left first metatarsal, wherein interaction with a DP plate and blade placements generally represented by reference numeral 110 and utilized in a ROSE bunionectomy procedure and/or Reverdin correction procedure is shown, according to the present disclosure. The Reverdin Jig 110 may comprise a medial Reverdin cut guide 111, a dorsal fixation wire insertion hole 112, an alignment mark 113, a dorsal Reverdin cut guide 114, a medial fixation wire insertion hole 115, positioning features 116, a dorsal plantar (DP) bone plate 117 (or dorsal bone plate), and blade placements 118.

In FIG. 11D, a top view of a Reverdin Jig fixated to the left first metatarsal, wherein interaction with a DP plate and blade placements generally represented by reference numeral 110 and utilized in a ROSE bunionectomy procedure and/or Reverdin correction procedure is shown, according to the present disclosure. The Reverdin Jig 110 may comprise a medial Reverdin cut guide 111, a dorsal fixation wire insertion hole 112, an alignment mark 113, a dorsal Reverdin cut guide 114, a medial fixation wire insertion hole 115, positioning features 116, a dorsal plantar (DP) bone plate 117 (or dorsal bone plate), and blade placements 118.

In FIG. 11E, a right side view of a Reverdin Jig fixated to the left first metatarsal, wherein interaction with a DP plate and a medial blade placement generally represented by reference numeral 110 and utilized in a ROSE bunionectomy procedure and/or Reverdin correction procedure is shown, according to the present disclosure. The Reverdin Jig 110 may comprise a medial Reverdin cut guide 111, a dorsal fixation wire insertion hole 112, an alignment mark 113, a dorsal Reverdin cut guide 114, a medial fixation wire insertion hole 115, positioning features 116, a dorsal plantar (DP) bone plate 117 (or dorsal bone plate), blade placements 118, and blade(s) 119.

In FIG. 11F, a right side view of a Reverdin Jig fixated to the left first metatarsal, wherein interaction with a DP plate and both medial and dorsal blade placements generally represented by reference numeral 110 and utilized in a ROSE bunionectomy procedure and/or Reverdin correction procedure is shown, according to the present disclosure. The Reverdin Jig 110 may comprise a medial Reverdin cut guide 111, a dorsal fixation wire insertion hole 112, an alignment mark 113, a dorsal Reverdin cut guide 114, a medial fixation wire insertion hole 115, positioning features 116, a dorsal plantar (DP) bone plate 117 (or dorsal bone plate), blade placements 118, and blade(s) 119.

If the Reverdin procedure is initiated after a ROSE correction, the Reverdin Jig placement is additionally determined by the relative location of the jig with the fixated dorsal or DP bone plate. A concave part on the proximal side of the Reverdin Jig follows the radiused distal end of the fixated bone plate.

Once properly placed, the Reverdin Jig can be fixated to the metatarsal bone in two dorsal locations by drilling Kirshner wires (e.g., K-wires), or similar, through each of two wire location holes in the near face of the jig. These fixation wires enter the jig, pass through the jig, thereby entering the dorsal face of the metatarsal bone. Countersinks on the near face of the jig, at the wire location holes, are intended to mate with the olive-style orthopedic wires, which are the preferred fixation wire type. This process is repeated for a single medial wire fixation. Further, the Reverdin Jig is mounted flush against the lateral side of the exposed first metatarsal.

### Executing the Reverdin Cut

With proper placement and fixation, the Reverdin Jig now clearly indicates and allows for proper positioning of the Reverdin wedge cuts.

The jig portion of a Reverdin Guide Clamp instrument is intended for use with a 10 mm wide, 0.5 mm thick sagittal saw blade, though the design can be readily modified for use with other readily available blade geometries. To begin, the surgeon uses the medial cut template of the jig portion of the instrument to fully cut through the metatarsal from the medial to lateral face. The saw and blade are removed, and a spare blade is placed within this cut.

The second cut starts from the dorsal surface of the metatarsal, guided by the dorsal cut template of the jig portion of the Reverdin Guide Clamp instrument. The sagittal saw blade cuts through the dorsal face of the metatarsal and is stopped by the spare blade previously placed within the medial cut. This prevents the action of the sagittal saw from inadvertent disturbance of the sesamoid bones. The surgeon continues to work the sagittal saw within the dorsal cut guide until a full wedge of bone is removed.

The Reverdin Jig can be utilized as a stand-alone orthopedic instrument, though the preferred use is incorporated into the Reverdin Guide Clamp.

### Reverdin Guide Clamp

The ROSE Bunion Correction system and method may comprise a Reverdin Guide Clamp instrument, which incorporates the Reverdin Jig described herein with a unique hinged clamp for consistent and reliable reproduction of the Reverdin correction. The hinged Reverdin Guide Clamp provides for robust clamping of the instrument to the metatarsal during preparation for and execution of the Reverdin correction. The folding feature of the hinged clamp enables the clamp handles to be rotationally repositioned (a.k.a., folded) to the distal or proximal location, providing sufficient clearance to allow the surgeon to make the Reverdin cut with a sagittal saw while the instrument remains clamped. Additionally, special features of the instrument described herein, when used in conjunction with a spare sagittal blade, provide protection to the sesamoid bones.

The rotational folding motion of the instrument is needed to enable the handles and body of the clamp to be moved out of the way so that they don't interfere and get in the way of the saw as it is inserted in the jig and cuts the bone. Further, this unique and novel feature of a hinge built into the clamp to enable the handles and body of the clamp to be repositioned in different directions to allow freedom from an intrinsic block, which would otherwise be caused by the handles and body of the clamp, has applications in surgical specialties of all disciplines.

The Reverdin Guide Clamp, with an integrated Reverdin Jig, was designed to readily determine proper positioning of the Reverdin correction (e.g., wedge) cut and correct a deviated articulated set angle. It can be used together with the ROSE bunionectomy procedure or as a standalone correction protocol.

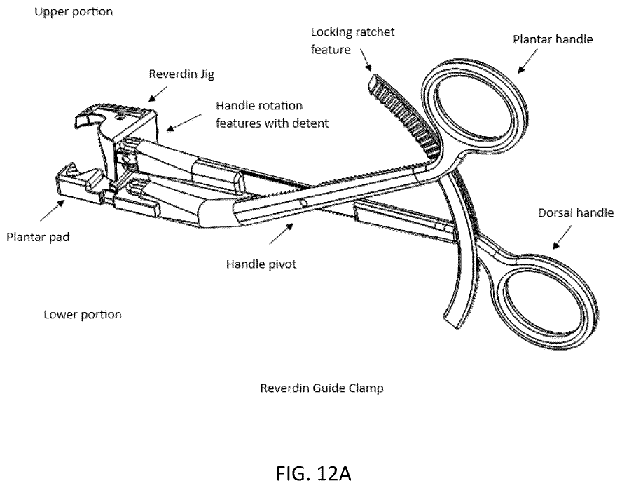

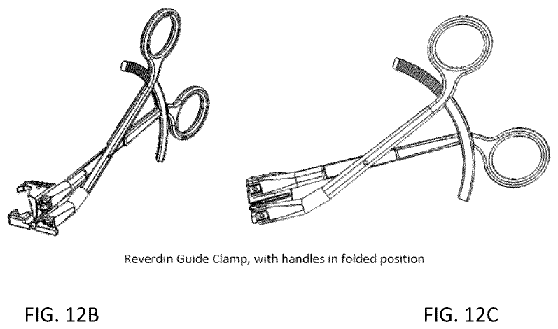

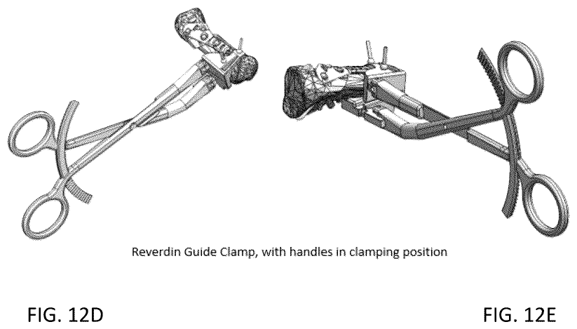

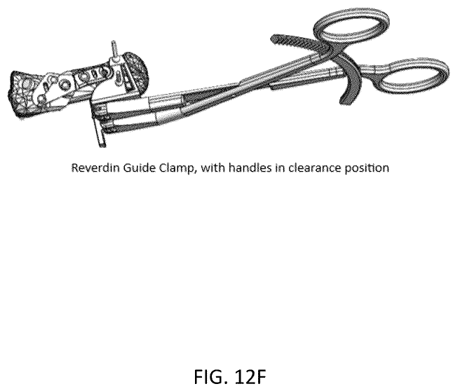

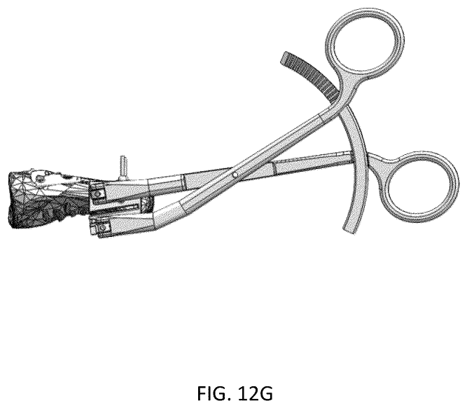

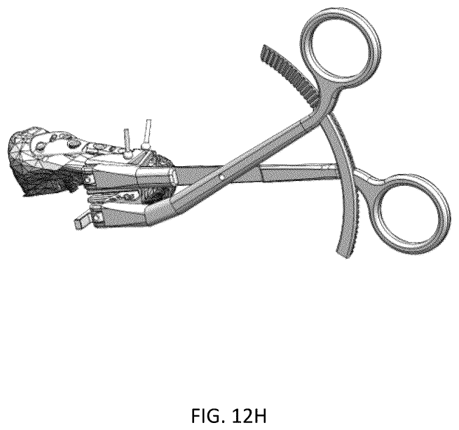

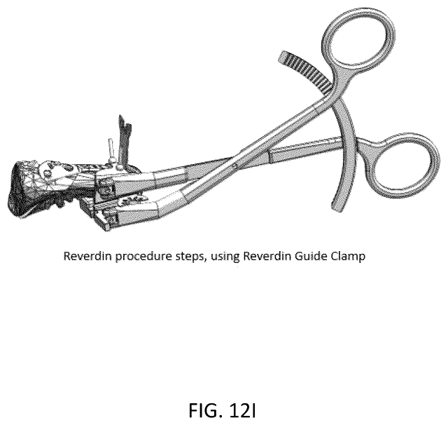

In FIG. 12A, a left side, perspective view of a Reverdin Guide Clamp with handles in a clamping position and with an integrated Reverdin Jig generally represented by reference numeral 120 and utilized in a ROSE bunionectomy procedure and/or Reverdin correction procedure is shown, according to the present disclosure. The Reverdin Guide Clamp 120 may comprise a plantar pad 121, a Reverdin Jig 122, a handle rotation features with detent 123, a locking ratchet feature 124, a plantar handle 125, a dorsal handle 126, and a handle pivot 127.

In FIGS. 12B and 12C, a front and side perspective views of a Reverdin Guide Clamp with handles in a cut clearance (a.k.a., “folded”) position and with an integrated Reverdin Jig generally represented by reference numeral 120 and utilized in a ROSE bunionectomy procedure and/or Reverdin correction procedure are shown, according to the present disclosure. The Reverdin Guide Clamp 120 may comprise a plantar pad 121, a Reverdin Jig 122, a handle rotation features with detent 123, a locking ratchet feature 124, a plantar handle 125, a dorsal handle 126, and a handle pivot 127.

### Placement of the Reverdin Guide Clamp

The Reverdin Guide Clamp may comprise dorsal jig positioning features geometrically designed to mate with anatomic bone contours, plantar pad positioning features, clamp handles with locking ratchet features to securely clamp the dorsal and plantar positioning features to the exposed metatarsal, wire fixation features, a medial slot feature to properly guide a sagittal saw blade cut fully through the metatarsal bone with a medial insertion to lateral exit, and a dorsal slot feature to guide a sagittal saw blade cut partially through the metatarsal bone from a dorsal insertion from the medial face to 60% through to the lateral face, while protecting the sesamoid bones by inserting a spare sagittal blade through the medial jig slot and cut.

To utilize the Reverdin Guide Clamp instrument, the Reverdin jig is first positioned using surgeon markings and/or anatomical features on the first metatarsal bone. This may include a mark applied by the surgeon to the midline dorsal surface of the exposed metatarsal bone prior to jig placement, which is then aligned with markings on the jig portion of the instrument. Additionally, the jig and plantar pad may comprise organically designed bosses and/or surfaces to contact and mate with typical bone surface geometry.

If the Reverdin procedure is initiated after a ROSE correction, the Reverdin Guide Clamp instrument placement is additionally determined by the relative location of a jig cut-out with the fixated dorsal or DP bone plate. A concave feature on the proximal side of the Reverdin Jig follows the radiused distal end of the fixated bone plate.

### Fixturing of the Reverdin Guide Clamp

The Reverdin Guide Clamp device may comprise a rotationally folding hinge design that connects the lower handle to the dorsal jig portion of the upper clamp (i.e., upper hinge). A similar hinge design connects the upper handle to the plantar pad portion of the lower clamp (i.e., lower hinge). To maximize clamp force, the axis of the upper hinge is essentially collinear to the axis of the lower hinge.

The Reverdin Guide Clamp device handles can be rotated in unison with the hinge axis as the center of rotation. Detents within the hinge assembly, with one device embodiment incorporating spring loaded detents, allow the device handles to be retained in set angular positions including but not limited to a distal position, clamping position, and proximal position.

To clamp the Reverdin Guide Clamp device, the handles are set in a position essentially perpendicular to the lateral plane of the metatarsal. When the device handles are squeezed together, the hinges transmit the applied force to clamp the dorsal jig portion and plantar pad securely against the metatarsal. The jig and pad features are designed to conform to specific anatomical contours of the metatarsal, further reinforcing the proper placement and retention of the device.

Once properly placed, the jig portion of the Reverdin Guide Clamp can be additionally fixated to the metatarsal bone in two dorsal and/or one medial locations by drilling Kirshner wires, or similar, through each of three wire location holes in the near face of the jig portion. These fixation wires enter the Reverdin Jig, pass through the jig, enter the dorsal and/or medial face of the metatarsal bone. Countersinks on the near face of the jig, at the wire location holes, are intended to mate with the olive-style orthopedic wires, which is the preferred fixation wire type. The Reverdin Jig is now mounted flush against the dorsal and medial side of the exposed first metatarsal.

### Folding Handles for Cut Clearance

The Reverdin Guide Clamp device handles can be rotated in unison with the hinge axis as the center of rotation. Detents within the hinge assembly, with one device embodiment incorporating spring loaded detents, allow the device handles to be retained in set angular positions including but not limited to a distal position, clamping position, and proximal position. To provide sufficient clearance for the Reverdin wedge cuts, the rotationally folding handles are placed in the distal position.

With proper placement, fixation, clamping and folding, the Reverdin Guide Clamp clearly indicates and allows for proper positioning of the Reverdin wedge cuts.

### Executing the Reverdin Cut

The jig portion of the Reverdin Guide Clamp instrument is intended for use with a 10 mm wide, 0.5 mm thick sagittal saw blade, though the design can be readily modified for use with other readily available blade geometries. To begin, the surgeon uses the medial cut template of the jig portion of the instrument to fully cut through the metatarsal from the medial to lateral face. The saw and blade are removed, and a spare blade is placed within this cut.

The second cut starts from the dorsal surface of the metatarsal, guided by the dorsal cut template of the jig portion of the Reverdin Guide Clamp instrument. The sagittal saw blade cuts through the dorsal face of the metatarsal and is stopped by the spare blade previously placed within the medial cut. This prevents the action of the sagittal saw from inadvertent disturbance of the sesamoid bones. The surgeon continues to work the sagittal saw within the dorsal cut guide until a full wedge of bone is removed.

After the Reverdin wedge cuts are completed, the rotationally folded handles are returned to the clamping position. Retention wires, if any, are removed. The handle ratchet features are released, and the instrument is removed from the metatarsal.

### Executing and Fixating the Reverdin Cut

The surgeon is now able to complete the Reverdin correction. The Reverdin Jig is removed, and the wedge closed by feathering the lateral dorsal cortex. In addition, if IM closure or further IM closure is necessary, the lateral cortex can be penetrated after feathering the IM closed and the capital fragment translated until the desired IM reduction is achieved. The Reverdin correction is secured with a 3.0 mm, or other size cannulated headless double threaded screw.

In FIGS. 12D and 12E, a top, right side and left side perspective views of a Reverdin Guide Clamp with handles in a clamping position and with an integrated Reverdin Jig generally represented by reference numeral 120 and utilized in a ROSE bunionectomy procedure and/or Reverdin correction procedure are shown, according to the present disclosure. The Reverdin Guide Clamp 120 may comprise a plantar pad 121, a Reverdin Jig 122, a handle rotation features with detent 123, a locking ratchet feature 124, a plantar handle 125, a dorsal handle 126, a handle pivot 127, a dorsal bone plate 128 (or dorsal plantar (DP) bone plate), and blade placements 129.

In FIG. 12F, a Reverdin Guide Clamp with handles in a cut clearance position and with an integrated Reverdin Jig generally represented by reference numeral 120 and utilized in a ROSE bunionectomy procedure and/or Reverdin correction procedure is shown, according to the present disclosure. The Reverdin Guide Clamp 120 may comprise a plantar pad 121, a Reverdin Jig 122, a handle rotation features with detent 123, a locking ratchet feature 124, a plantar handle 125, a dorsal handle 126, a handle pivot 127, a dorsal bone plate 128 (or dorsal plantar (DP) bone plate), and blade placements 129.

In FIG. 12G, a side view of a Reverdin Guide Clamp utilized in the Reverdin procedure steps and with an integrated Reverdin Jig generally represented by reference numeral 120 and utilized in a ROSE bunionectomy procedure and/or Reverdin correction procedure is shown, according to the present disclosure. The Reverdin Guide Clamp 120 may comprise a plantar pad 121, a Reverdin Jig 122, a handle rotation features with detent 123, a locking ratchet feature 124, a plantar handle 125, a dorsal handle 126, a handle pivot 127, a dorsal bone plate 128 (or dorsal plantar (DP) bone plate), and blade placements 129.

In FIG. 12H, a left side, perspective view of a Reverdin Guide Clamp utilized in the Reverdin procedure steps and with an integrated Reverdin Jig generally represented by reference numeral 120 and utilized in a ROSE bunionectomy procedure and/or Reverdin correction procedure is shown, according to the present disclosure. The Reverdin Guide Clamp 120 may comprise a plantar pad 121, a Reverdin Jig 122, a handle rotation features with detent 123, a locking ratchet feature 124, a plantar handle 125, a dorsal handle 126, a handle pivot 127, a dorsal bone plate 128 (or dorsal plantar (DP) bone plate), and blade placements 129.

In FIG. 12I, a front, side view of a Reverdin Guide Clamp utilized in the Reverdin procedure steps and with an integrated Reverdin Jig generally represented by reference numeral 120 and utilized in a ROSE bunionectomy procedure and/or Reverdin correction procedure is shown, according to the present disclosure. The Reverdin Guide Clamp 120 may comprise a plantar pad 121, a Reverdin Jig 122, a handle rotation features with detent 123, a locking ratchet feature 124, a plantar handle 125, a dorsal handle 126, a handle pivot 127, a dorsal bone plate 128 (or dorsal plantar (DP) bone plate), blade placements 129, and blade(s) 1200.

### Dorsal Bone Plate

The ROSE bunionectomy procedure may comprise at least two plate options—a dorsal bone plate and a dorsal plantar (DP) bone plate, according to the present disclosure. The dorsal bone plate contacts the corrected metatarsal on the dorsal surface only, while the DP bone plate contacts the corrected metatarsal on dorsal, medial, and plantar surfaces.

The near surface of the plate is essentially convex, while the far surface of the plate (e.g., which contacts the surface of the metatarsal) is essentially concave. The plate is specifically designed to mate with typical anatomical contours of the metatarsal, allowing for the proper placement and firm retention of the implant.

The dorsal bone plate can be used with or without the ROSE Fixation Clamp, though the preference is to utilize this optimized plate with the instrument to ensure proper positioning of the plate on the corrected metatarsal.

Dorsal Bone Plate Placement without the Fixation Clamp

When used without the ROSE Fixation Clamp, the dorsal plate is positioned in part by utilizing a plate marking that indicates the dorsal distal exit point of the ROSE osteotomy cut. Additional placement guidance comes from the anatomical wrap of the plate from the dorsal to medial faces of the bone.

At least two 2.4 mm, or other sized locking or non-locking screws are inserted through the screw holes of the Dorsal bone plate. In one embodiment of the Dorsal bone plate, the screw clearance holes may include a threaded portion to mate with a threaded drill guide (a.k.a., tower). Further, the locking or non-locking screws may be inserted with the aid of a threaded drill guide.

Additionally, locking or non-locking screw holes are located and angled to guide locking or non-locking screw insertion and fixation to ensure the locking or non-locking screws are sufficiently spaced away from the osteotomy surface edge (e.g., at the outer bone surface interface), will not interfere with other fixation locking or non-locking screws, and are sufficiently spaced from other locking or non-locking screws to alleviate any stress. The series of locking or non-locking screws includes fixations across the osteotomy cut, as well as fixations within the dorsal fragment only and within the plantar fragment only, to minimize effective ground reaction force at the bone fragment interface (i.e., neutralize).

Dorsal Bone Plate Fixation Placement with the ROSE Fixation Clamp

When used with the ROSE Fixation Clamp, the two through slots of the plate engage and are temporarily retained by two plate fixturing bosses on the clamp. The dorsal bone plate is then clamped on the corrected metatarsal between the Fixation Clamp dorsal and plantar pads. Proper positioning of the plate on the corrected metatarsal is guided by markings on the Fixation Clamp that indicate the relative position of the osteotomy cut, contoured features on the dorsal bone plate that mate typical anatomical features on the dorsal and medial surfaces of the dorsal metatarsal fragment, as well as anatomical features on the dorsal surface of the plantar metatarsal fragment, and contoured features on the plantar pad of the Fixation Clamp.

The ROSE Fixation Clamp is securely clamped by squeezing the handles. Ratcheting features on the instrument handles retain the secure clamping.

At least two 2.4 mm, or other sized locking or non-locking screws are inserted through the screw clearance holes of the ROSE Fixation Clamp. In one embodiment of the ROSE Fixation Clamp, the screw clearance holes may include a threaded portion to mate with a threaded drill guide (a.k.a., tower). The locking or non-locking screws may be inserted with the aid of a threaded drill guide. Once these two locking or non-locking screws are securely fastened, the clamp is removed from the metatarsal by releasing the ratcheting features on the instrument handles. The remaining locking or non-locking screws are inserted.

Additionally, locking or non-locking screw clearance hole are located and angled to guide locking or non-locking screw insertion and fixation to ensure the locking or non-locking screws are sufficiently spaced away from the osteotomy surface edge (e.g., at the outer bone surface interface), will not interfere with other fixation locking or non-locking screws, and are sufficiently spaced from other locking or non-locking screws to alleviate any stress. The series of locking or non-locking screws includes fixations across the osteotomy cut, as well as fixations within the dorsal fragment only and within the plantar fragment only, to minimize effective ground reaction force at the bone fragment interface (i.e., neutralize).

Additional Dorsal Bone Clamp embodiments

In one embodiment of a Dorsal bone clamp described herein, the locking or non-locking screw clearance holes may include a threaded portion to accept a standard orthopedic threaded drill guide (a.k.a., tower) to ensure proper directional placement of the locking or non-locking screws.

In one embodiment of the Dorsal bone plate, the bone plate may fixate a range of intermetatarsal angle corrections. Additional bone plate designs can uniquely incorporate the corrected anatomical surfaces and/or locking or non-locking screws relative positions to properly fixate another range of intermetatarsal angle corrections. These ranges may overlap and are expected to cover intermetatarsal correction angles from 5 to 20 degrees.

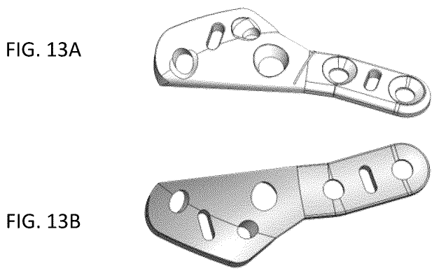

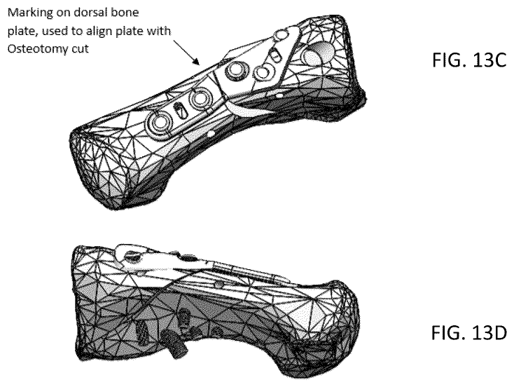

In FIGS. 13A and 13B, a top and bottom perspective views of a dorsal bone plate having a marking on the dorsal surface used to align the bone plate with an osteotomy cut generally represented by reference numeral 130 and utilized in a ROSE bunionectomy procedure are shown, according to the present disclosure. The dorsal bone plate 130 may comprise an alignment marking 131, locking or non-locking screw clearance holes 132, and a threaded portion 133.

In FIGS. 13C and 13D, a top and bottom perspective views of a dorsal bone plate secured to the dorsal surface of a corrected metatarsal generally represented by reference numeral 130 and utilized in a ROSE bunionectomy procedure are shown, according to the present disclosure. The dorsal bone plate 130 may comprise an alignment marking 131, locking or non-locking screw clearance holes 132, a threaded portion 133, a dorsal metatarsal fragment 1300, and a plantar metatarsal fragment 1301.

Dorsal Plantar (DP) bone plate

The ROSE bunionectomy procedure may comprise at least two plate options—a dorsal bone plate and a dorsal plantar (DP) bone plate, according to the present disclosure. The dorsal bone plate contacts the corrected metatarsal on the dorsal surface only, while the DP bone plate contacts the corrected metatarsal on dorsal, medial, and plantar surfaces.

The DP bone plate can be sized appropriately and used in bunion correction applications as well as for applications in at least long bones beyond bunion correction, where a fracture and/or osteotomy is oblique and unstable. Further, this DP bone plate approach can be used with any long bone of the body. This includes oblique fractures and/or osteotomies of the lower extremities, including but not limited to a femur, tibia, and fibula, as well as applications for upper extremities, including but not limited to a humerus, ulna, radius and metacarpals.

The DP plate's curved geometry and internally threaded boss are designed to guide a plate clamping screw through the osteotomy and/or fracture area to introduce additional stabilization by clamping the fracture and/or osteotomy, and thereby compressing the fragments as the novel plate clamping screw passes through the plate and engages with the internally threaded boss. As the plate wraps around the bone, this integrated threaded clamping creates additional stability as compared to conventional plates.

Furthermore, unlike conventional plates, the unique DP bone plate integrates the remaining screws originating from bidirectional planes, thereby neutralizing the forces acting on the osteotomy and/or fracture. This allows for very early loading (i.e., weight bearing), as well as range of motion, since the plate absorbs the forces and weight bearing displacement by its clamping and bidirectionality of the remaining screws in the plate, where otherwise prolonged immobilization may have been necessary.

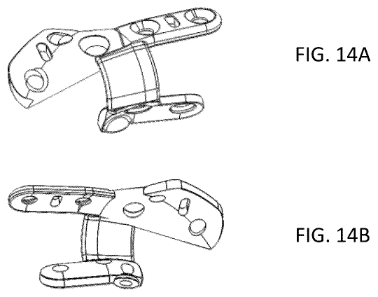

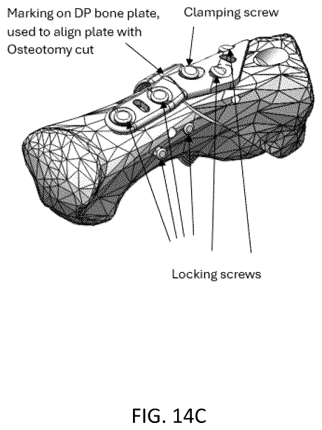

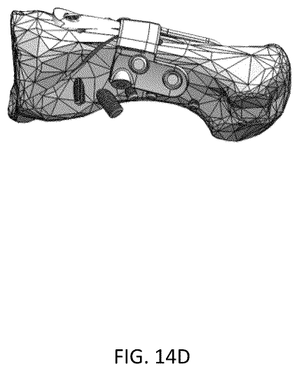

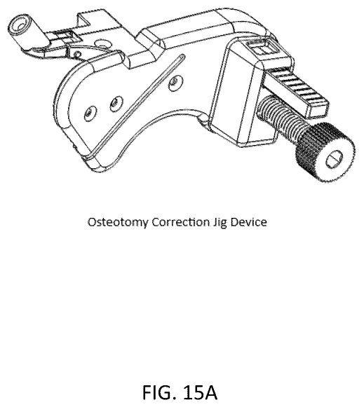

In FIGS. 14A and 14B, a top and bottom perspective views of a dorsal plantar (DP) bone plate having a marking on the dorsal surface used to align the bone plate with an osteotomy cut and an integrated internally threaded boss to accept a clamping screw generally represented by reference numeral 140 and utilized in a ROSE bunionectomy procedure are shown, according to the present disclosure. The dorsal bone plate 140 may comprise an internally threaded boss 141, alignment marking 142, locking or non-locking screw clearance holes 143, and a threaded portion 143.

The near surface of the plate is essentially convex, while the far surface of the plate (e.g., which contacts the surface of the metatarsal) is essentially concave. The plate is specifically designed to mate with typical anatomical contours of the metatarsal, allowing for the proper placement and firm retention of the implant.

Currently, bunionectomy fixations are performed using dorsal bone plates or plates with some, though limited, curvature that follows the anatomical markers of the first metatarsal.

The ROSE Bunion Correction system and method may comprise the option of a DP bone plate that securely fixates the dorsal and plantar fragments simultaneously. The unique and novel DP bone plate extends further around the corrected metatarsal than current bone plates. The design of the plate allows the surgeon to fixate some bone screws in a perpendicular orientation to the osteotomy cut for improved, more efficient compression and an expectation for improved force (e.g., weight) bearing over the osteotomy cut surfaces.

In FIG. 14C, a top, perspective view of a dorsal plantar (DP) bone plate having a marking on the DP bone plate, used to align the bone plate with an Osteotomy cut generally represented by reference numeral 140 and utilized in a ROSE bunionectomy procedure is shown, according to the present disclosure. The DP bone plate 140 may comprise an internally threaded boss 141, alignment marking 142, locking or non-locking screw clearance holes 143, a threaded portion 143, a clamping screw 144, and locking or non-locking screws 145, a dorsal metatarsal fragment 1500, and a plantar metatarsal fragment 1501.

In FIG. 14D, a bottom, perspective view of a fixated dorsal plantar (DP) bone plate having a marking on the DP bone plate with a clamping screw installed within an internally threaded boss, generally represented by reference numeral 140 and utilized in a ROSE bunionectomy procedure is shown, according to the present disclosure. The DP bone plate 140 may comprise an internally threaded boss 141, alignment marking 142, locking or non-locking screw clearance holes 143, a threaded portion 143, a clamping screw 144, and locking or non-locking screws 145, a dorsal metatarsal fragment 1500, and a plantar metatarsal fragment 1501.

The DP bone plate may comprise a dorsal portion, a plantar portion, and a connection web.

DP Bone Plate Fixation Placement with the ROSE Fixation Clamp

When the DP bone plate is used in conjunction with the ROSE Fixation Clamp, the two through slots of the plate engage with and is temporarily retained by two plate fixturing bosses on the clamp. The DP bone plate is then clamped on the corrected metatarsal between the Fixation Clamp dorsal and plantar pads. Proper positioning of the plate on the corrected metatarsal is guided by markings on the Fixation Clamp that indicate the relative position of the osteotomy cut, contoured features on the DP bone plate that mate typical anatomical features on the dorsal and medial surfaces of the dorsal metatarsal fragment, as well as anatomical features on the dorsal surface of the plantar metatarsal fragment, contoured features on the DP bone plate that mate typical anatomical features on the plantar surfaces of the plantar metatarsal fragment, and contoured features on the plantar pad of the Fixation Clamp.

The ROSE Fixation Clamp is securely clamped by squeezing the handles. Ratcheting features on the instrument handles retain the secure clamping.

### Ensuring Alignment of Dorsal-to-Plantar Compression Screw

The DP bone plate provides additional clamping effect with a non-locking clamping bone screw that passes through the dorsal portion of the DP bone plate and securely threads (e.g., fastens) into an axially aligned internally threaded boss on the plantar portion of the plate.

To ensure this proper axial alignment, a threaded Kirshner wire (e.g., K-wire), or similar drill guide is inserted into the proximal screw clearance hole on the ROSE Fixation Clamp's dorsal pad. Further, a K-wire is drilled through the Fixation Clamp, dorsal fragment and plantar fragment. The K-wire should exit cleanly through the internally threaded boss on the DP bone plate. If needed, the clamp may be readjusted, and the wire redrilled. Once the alignment is verified, the threaded K-wire drill guide is removed and a 2.4 mm, or other size cannulated clamping bone screw is inserted over the guide wire to engage the threads of the clamping screw into the internally threaded boss to create the clamping effect of the DP plate.

A 2.4 mm, or other size locking or non-locking screw is inserted through the distal screw clearance hole on the ROSE Fixation Clamp's dorsal pad. In one embodiment of the ROSE Fixation Clamp, the screw clearance holes may include a threaded portion to mate with a threaded drill guide. This may be inserted with the aid of a threaded drill guide. Once this locking or non-locking screw is securely fastened, the clamp is removed from the metatarsal by releasing the ratcheting features on the instrument handles.

The remaining locking or non-locking screws are inserted. Locking or non-locking screw clearance holes are located and angled to guide locking or non-locking screw insertion and fixation to ensure the locking or non-locking screws are sufficiently spaced away from the osteotomy surface edge (e.g., at the outer bone surface interface), will not interfere with other fixation locking or non-locking screws, and are sufficiently spaced from other locking or non-locking screws to alleviate any stress. The series of locking or non-locking screws includes fixations across the osteotomy cut, as well as fixations within the dorsal fragment only and within the plantar fragment only, to maximize compression.

Note: A surgeon may choose to not take advantage of the Dorsal-to-Plantar clamping screw, which traverses the DP plate, but rather to simply fixate the DP bone plate with a series of dorsal and plantar locking or non-locking screws, which traverse either the dorsal or plantar portion of the DP bone plate, but not both portions.

### Additional Embodiments

In one embodiment of the DP bone clamp, all locking or non-locking screw clearance holes may include a threaded portion to accept a standard orthopedic threaded drill guide (a.k.a., tower) to ensure proper directional placement of the locking or non-locking screws.

In one embodiment of the DP bone plate design, the bone plate may fixate a range of intermetatarsal angle corrections. Additional bone plate designs can uniquely incorporate the corrected anatomical surfaces and/or locking or non-locking screws relative positions to properly fixate another range of intermetatarsal angle corrections. These ranges may overlap and are expected to cover intermetatarsal correction angles from 5 to 20 degrees.

Additional Surgical Clamps with Folding Handles

The rotational folding handles of the ROSE Guide Clamp can be readily applied to other clamps within the ROSE Bunion Correction system and method, such as the Reverdin Guide Clamp. For the Reverdin Guide Clamp, as with the ROSE Guide Clamp, the folding handles allow the surgeon to apply the clamping force required during device placement, and once rotationally folded distally (or proximally if appropriate), provides the physical clearance necessary to provide the surgeon with a maximized field of view and prevent instrument interference (e.g., sagittal saw and clamp handles).

Additionally, the rotational folding handles of the ROSE Guide Clamp can be readily applied to other clamps outside the ROSE Bunion Correction system and method, such as the standard bone clamps.

ROSE Osteotomy Correction Jig device (a.k.a., Osteotomy Correction Jig assembly)

The ROSE Bunion Correction system and method may comprise a novel ROSE Osteotomy Correction Jig device (a.k.a., “Osteotomy Correction Jig assembly”), which includes features to execute the Osteotomy, correct the Intermetatarsal (IM) angle, and retain the IM angle correction without the need for removal of the Osteotomy Correction Jig device from the metatarsal, as described herein.

In other words, the ROSE Osteotomy Correction Jig assembly is secured to the metatarsal throughout the entire osteotomy and correction process, and partially during the fixation process. By retaining the device throughout most of the procedure (i.e., sans final fixation with bone plate), it effects a more streamlined and repeatable procedure while minimizing the loss of desired correction in the time between correction and final fixation.

The Rotational Osteotomy System for Enhanced (ROSE) Bunion Correction is a set of novel instruments and implants to repeatedly and robustly perform the novel and unique osteotomy cut and bunion correction system developed by Dr. Tzvi Bar-David. The procedure may additionally benefit from Dr. Bar-David's novel instrument for Reverdin correction, a procedure that can be implemented as the optional final step of a ROSE correction, or as a stand-alone procedure.

This present disclosure describes herein the design features and benefits of the ROSE Osteotomy Correction Jig device. With its novel design and integrated features, the Osteotomy Correction Jig device is a unique and integral tool utilized in the ROSE Bunion Correction system and method.

### Additional Characteristics:

The ROSE Osteotomy Correction Jig device has a left-hand and a right-hand version. The left-hand version is a true mirror of the right-hand version.

The ROSE bunionectomy procedure utilizes additional instruments and implants already known in the arts including sagittal bone saws and blades, bone drills, Kirshner wires threaded and non-threaded, olive wires threaded and non-threaded, locking or non-locking bone screws, cannulated bone screws, bone clamps including Verbrugge-style bone clamps, and similar.

ROSE Osteotomy Correction Jig device

The ROSE bunionectomy procedure provides benefits over other bunion correction protocols by including but not limited to (1) increasing or reducing the first intermetatarsal angle without a change in overall metatarsal length and (2) providing an optimized fixated fragment interface to assist in post ambulatory recovery.

These dual benefits are driven, in part, by the perpendicular relationship between the metatarsal dorsal and plantar fragment rotational axis and the osteotomy cut plane, which in turn becomes the contact interface between the fixated dorsal and plantar fragments. This optimized interface has a maximized surface area and is located on a plane that provides, once fixated, optimal support against typical post-ambulatory ground reaction force.

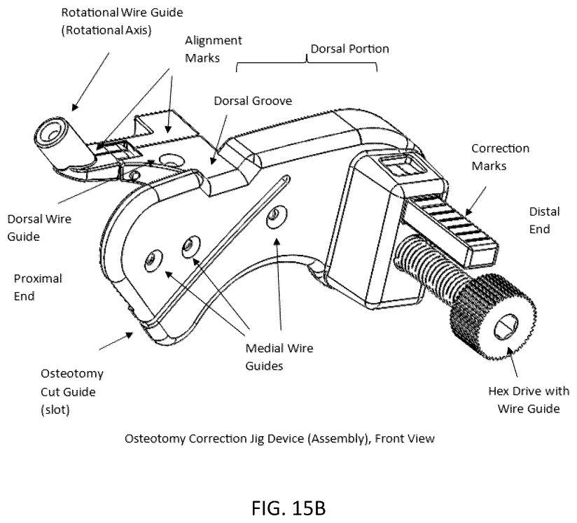

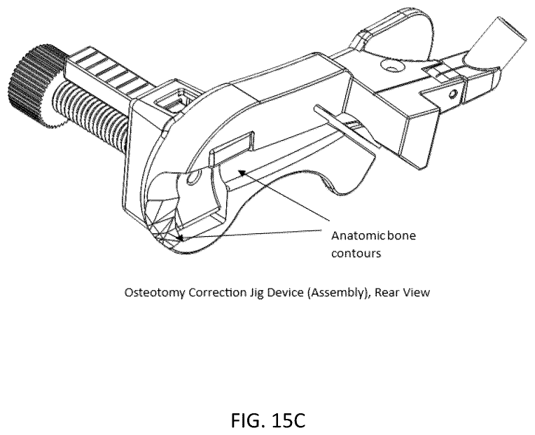

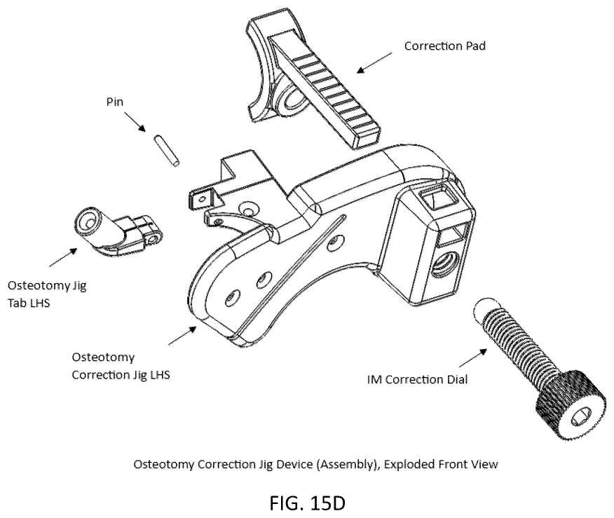

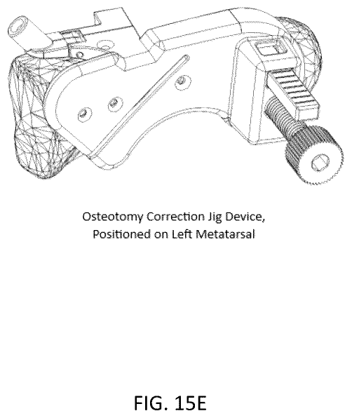

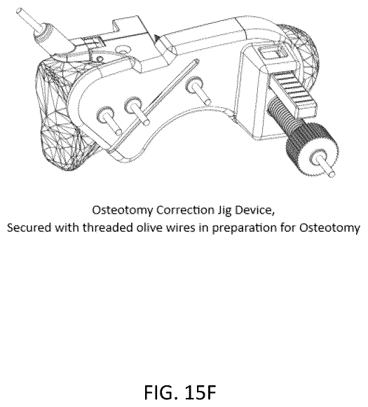

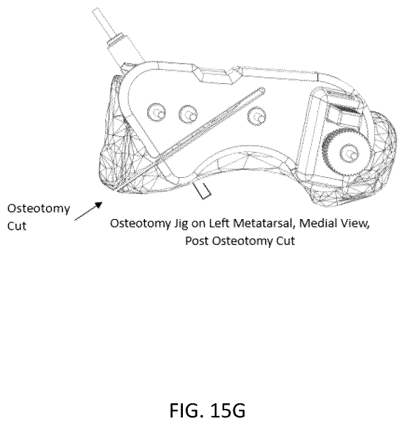

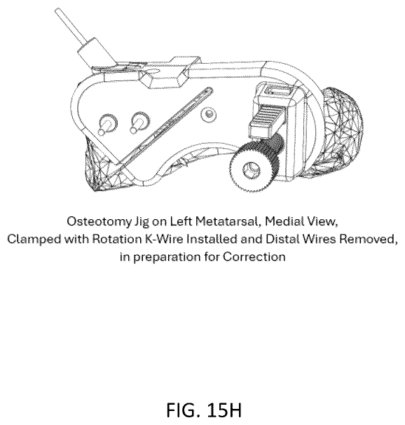

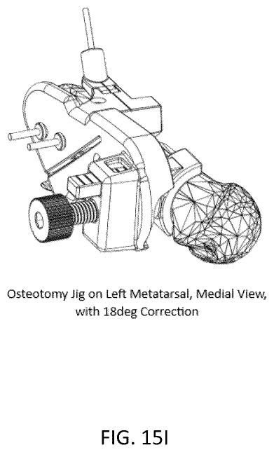

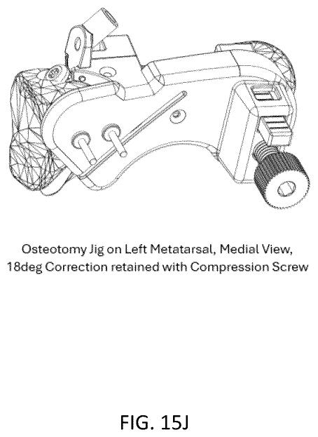

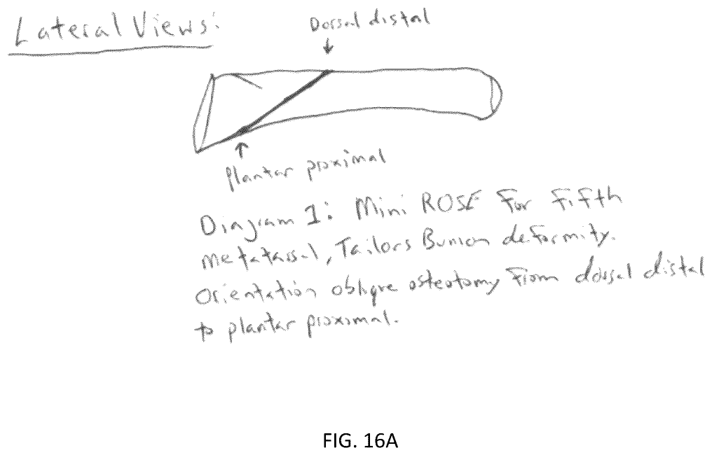

In FIG. 15A, a perspective view of a ROSE Osteotomy Correction Jig device generally represented by reference numeral 150 and utilized in a ROSE bunionectomy procedure is shown, according to the present disclosure. The Osteotomy Correction Jig assembly 150 may comprise an osteotomy cut guide (slot) 151, a dorsal wire guide 152, a rotational wire guide (rotational axis) 153, alignment marks 154, a dorsal groove 155, correction marks 156, a hex drive with wire guide 157, medial wire guides 158, a proximal end 1500, and a distal end 1501.

In FIG. 15B, a front view of a ROSE Osteotomy Correction Jig device generally represented by reference numeral 150 and utilized in a ROSE bunionectomy procedure is shown, according to the present disclosure. The Osteotomy Correction Jig assembly 150 may comprise an osteotomy cut guide (slot) 151, a dorsal wire guide 152, a rotational wire guide (rotational axis) 153, alignment marks 154, a dorsal groove 155, correction marks 156, a hex drive with wire guide 157, medial wire guides 158, a proximal end 1500, and a distal end 1501.

In FIG. 15C, a rear view of a ROSE Osteotomy Correction Jig device generally represented by reference numeral 150 and utilized in a ROSE bunionectomy procedure is shown, according to the present disclosure. The Osteotomy Correction Jig assembly 150 may comprise an osteotomy cut guide (slot) 151, a dorsal wire guide 152, a rotational wire guide (rotational axis) 153, alignment marks 154, a dorsal groove 155, correction marks 156, a hex drive with wire guide 157, medial wire guides 158, anatomic bone contours 159, a proximal end 1500, and a distal end 1501.

In FIG. 15D, an exploded, front view of a ROSE Osteotomy Correction Jig device generally represented by reference numeral 150 and utilized in a ROSE bunionectomy procedure is shown, according to the present disclosure. The Osteotomy Correction Jig assembly 150 may comprise an osteotomy cut guide (slot) 151, a dorsal wire guide 152, a rotational wire guide (rotational axis) 153, alignment marks 154, a dorsal groove 155, correction marks 156, a hex drive with wire guide 157, medial wire guides 158, a proximal end 1500, a distal end 1501, an osteotomy correction jig LHS (e.g., left-hand side) 1502, an osteotomy jig tab LHS (e.g., left-hand side) 1503, a pin 1504, a correction pad 1505, and an IM correction dial 1506.

The ROSE Osteotomy Correction Jig device 150 was designed to readily determine proper positioning of the osteotomy cut and relative location of the rotational axis. The device may comprise positioning features geometrically designed to position with anatomic bone contours, features to accept and guide fixation wires, a slot feature to properly guide a sagittal saw blade fully through the metatarsal bone from a dorsal distal insertion to a plantar proximal exit without cartilage damage, and a manipulative guide feature to perpendicularly guide a Kirshner wire (e.g., K-wire), or similar, towards and/or through the osteotomy cut that can be manipulated to provide clearance for a fixation screw, a correction assembly to relatively rotate the dorsal and plantar fragments to the desired intermetatarsal angle, and a measurement guide to aid the surgeon in the correction.

### Position

In FIG. 15E, a perspective view of a ROSE Osteotomy Correction Jig device positioned on a left first metatarsal generally represented by reference numeral 150 and utilized in a ROSE bunionectomy procedure is shown, according to the present disclosure. The Osteotomy Correction Jig assembly 150 may comprise an osteotomy cut guide (slot) 151, a dorsal wire guide 152, a rotational wire guide (rotational axis) 153, alignment marks 154, a dorsal groove 155, correction marks 156, a hex drive with wire guide 157, medial wire guides 158, a proximal end 1500, a distal end 1501, an osteotomy correction jig LHS (e.g., left-hand side) 1502, an osteotomy jig tab LHS (e.g., left-hand side) 1503, a pin 1504, a correction pad 1505, an IM correction dial 1506, and a metatarsal 1507.

To utilize this device, the ROSE Osteotomy Correction Jig device is first positioned using surgeon markings and/or anatomical features on the first metatarsal bone. This may include a mark applied by the surgeon to the mid-line dorsal surface of the exposed metatarsal bone prior to device placement, which is then aligned with markings on the dorsal portion of the device. This may also include surgeon marks to indicate the desired plantar exit of the osteotomy. Additionally, the device may comprise organically designed bosses and surfaces to contact and mate with typical bone surface geometry.

### Secure

In FIG. 15F, a perspective view of a ROSE Osteotomy Correction Jig device positioned on a left first metatarsal and secured with threaded olive wires in preparation for an Osteotomy generally represented by reference numeral 150 and utilized in a ROSE bunionectomy procedure is shown, according to the present disclosure. The Osteotomy Correction Jig assembly 150 may comprise an osteotomy cut guide (slot) 151, a dorsal wire guide 152, a rotational wire guide (rotational axis) 153, alignment marks 154, a dorsal groove 155, correction marks 156, a hex drive with wire guide 157, medial wire guides 158, a proximal end 1500, a distal end 1501, an osteotomy correction jig LHS (e.g., left-hand side) 1502, an osteotomy jig tab LHS (e.g., left-hand side) 1503, a pin 1504, a correction pad 1505, an IM correction dial 1506, a metatarsal 1507, and threaded olive wires 1508.

Once properly positioned, the ROSE Osteotomy Correction Jig device/assembly is secured to the metatarsal with a combination of dorsal and medial wires. The dorsal portion is secured by drilling a threaded olive wire through the rotational wire guide. Additionally, the device can be secured to the metatarsal bone in four medial locations by inserting threaded or unthreaded olive wires, or similar, through each of three wire location holes in the near face of the jig. The fourth wire is drilled through the center hole guide of the Intermetatarsal (IM) Correction Dial.

These fixation wires enter the ROSE Osteotomy Correction Jig device, pass through the device, and enter the medial face of the metatarsal. Countersinks on the top surface of the tab component and near the face of the jig portion, at the wire location holes, are intended to mate with olive-style orthopedic wires, which are the preferred fixation wire type. Further, the device is mounted flush against the dorsal and medial side of the exposed first metatarsal.

### Cut

In FIG. 15G, a medial view of a ROSE Osteotomy Correction Jig device positioned on a left first metatarsal, secured with threaded olive wires, and after an Osteotomy generally represented by reference numeral 150 and utilized in a ROSE bunionectomy procedure is shown, according to the present disclosure. The Osteotomy Correction Jig assembly 150 may comprise an osteotomy cut guide (slot) 151, a dorsal wire guide 152, a rotational wire guide (rotational axis) 153, alignment marks 154, a dorsal groove 155, correction marks 156, a hex drive with wire guide 157, medial wire guides 158, a proximal end 1500, a distal end 1501, an osteotomy correction jig LHS (e.g., left-hand side) 1502, an osteotomy jig tab LHS (e.g., left-hand side) 1503, a pin 1504, a correction pad 1505, an IM correction dial 1506, a metatarsal 1507, threaded olive wires 1508, an osteotomy cut 1509, a dorsal metatarsal fragment 1510, and a plantar metatarsal fragment 1511.

Correctly placed and secured, the ROSE Osteotomy Correction Jig device/assembly clearly indicates and allows for proper positioning of the osteotomy cut. Thus, the surgeon can effectively execute the ROSE osteotomy cut.

The jig portion of the Osteotomy Correction Jig assembly may comprise an intuitive guide for the proper insertion at the distal dorsal end of the cut guide, controlled direction of the cut from the distal dorsal to plantar proximal end of the cut guide, and removal of the sagittal saw blade at the plantar proximal end of the cut guide. This provides for the proper execution of the ROSE osteotomy cut, with respect to the anatomy of the first (or fifth) metatarsal, as well as the correction axis of rotation, controlled later in the procedure by the placement of the drilled rotational wire guide.

Once the osteotomy cut has been successfully executed, the dorsal and plantar fragments are securely clamped in preparation for the full rotational K-wire insertion. This can be executed using a standard Verbrugge-style, or similar, bone clamp, based on the surgeon's discretion. The dorsal portion of the bone clamp is placed within the dorsal groove of the Osteotomy Correction Jig assembly, with care to ensure the plantar portion will provide sufficient clearance to the plantar exit of the rotational K-wire (inserted in next procedure step).

### Correct

In FIG. 15H, a medial view of a ROSE Osteotomy Correction Jig device positioned on a left first metatarsal, clamped with rotation K-wire installed and distal wires removed in preparation for bone correction generally represented by reference numeral 150 and utilized in a ROSE bunionectomy procedure is shown, according to the present disclosure. The Osteotomy Correction Jig assembly 150 may comprise an osteotomy cut guide (slot) 151, a dorsal wire guide 152, a rotational wire guide (rotational axis) 153, alignment marks 154, a dorsal groove 155, correction marks 156, a hex drive with wire guide 157, medial wire guides 158, a proximal end 1500, a distal end 1501, an osteotomy correction jig LHS (e.g., left-hand side) 1502, an osteotomy jig tab LHS (e.g., left-hand side) 1503, a pin 1504, a correction pad 1505, an IM correction dial 1506, a metatarsal 1507, threaded olive wires 1508, an osteotomy cut 1509, a dorsal metatarsal fragment 1510, a plantar metatarsal fragment 1511, and a rotational K-wire 1512.

With the metatarsal dorsal and plantar fragments securely clamped, the rotational K-wire can be fully inserted through both the dorsal and plantar fragments.

First, the dorsal threaded olive wire is removed. This is replaced with a rotational K-wire guided through the same wire guide features of the Osteotomy Correction Jig assembly tab, though fully drilled from the dorsal surface of the first metatarsal dorsal fragment through the plantar surface of the plantar fragment, perpendicularly crossing the osteotomy cut surfaces. In one embodiment of the ROSE instrument set, this rotational K-wire is color coded, or etched, to provide a visual indication to the surgeon of the fully inserted K-wire insertion depth.

In preparation for the IM correction, the two distal olive wires are removed and the Verbrugge-style, or similar, bone clamp is released slightly. The dorsal and plantar fragments can relatively rotate about the rotational K-wire, while maintaining a close planar positioning across the osteotomy cut.

In FIG. 15I, a medial view of a ROSE Osteotomy Correction Jig device positioned on a left first metatarsal, clamped with rotation K-wire installed and distal wires removed with an 18 degree bone correction generally represented by reference numeral 150 and utilized in a ROSE bunionectomy procedure is shown, according to the present disclosure. The Osteotomy Correction Jig assembly 150 may comprise an osteotomy cut guide (slot) 151, a dorsal wire guide 152, a rotational wire guide (rotational axis) 153, alignment marks 154, a dorsal groove 155, correction marks 156, a hex drive with wire guide 157, medial wire guides 158, anatomic bone contours 159, a proximal end 1500, a distal end 1501, an osteotomy correction jig LHS (e.g., left-hand side) 1502, an osteotomy jig tab LHS (e.g., left-hand side) 1503, a pin 1504, a correction pad 1505, an IM correction dial 1506, a metatarsal 1507, threaded olive wires 1508, an osteotomy cut 1509, a dorsal metatarsal fragment 1510, a plantar metatarsal fragment 1511, and a rotational K-wire 1512.

By gripping the knurled end of the IM Correction Dial, or with a hex driver, the surgeon rotates the IM Correction Dial to execute the desired correction. A correction pad, driven by the interface between the IM Correction Dial and internal threads of the jig portion, engages and moves the distal portion of the plantar fragment away from the jig face and rotates the fragment around the K-wire to the desired IM angle position. If needed, the surgeon can rotate the IM Correction Dial in the opposite direction to reduce or adjust the correction.

Once the desired correction is achieved, the Verbrugge-style, or similar, clamp is once again firmly clamped, in preparation for initial fixation.

### Retain

In FIG. 15J, a medial view of a ROSE Osteotomy Correction Jig device positioned on a left first metatarsal, clamped with rotation K-wire installed and proximal wires removed with an 18 degree bone correction, retained with a compression screw generally represented by reference numeral 150 and utilized in a ROSE bunionectomy procedure is shown, according to the present disclosure. The Osteotomy Correction Jig assembly 150 may comprise an osteotomy cut guide (slot) 151, a dorsal wire guide 152, a rotational wire guide (rotational axis) 153, alignment marks 154, a dorsal groove 155, correction marks 156, a hex drive with wire guide 157, medial wire guides 158, a proximal end 1500, a distal end 1501, an osteotomy correction jig LHS (e.g., left-hand side) 1502, an osteotomy jig tab LHS (e.g., left-hand side) 1503, a pin 1504, a correction pad 1505, an IM correction dial 1506, a metatarsal 1507, threaded olive wires 1508, an osteotomy cut 1509, a dorsal metatarsal fragment 1510, a plantar metatarsal fragment 1511, and a compression screw 1513 (e.g., 3.5 mm, or other size).

To retain the IM correction, the surgeon first removes the rotational K-wire from the dorsal rotational wire hole of the tab feature. This tab feature is then flipped upward away from the metatarsal distal surface. This provides clearance for compression screw insertion. At the surgeon's discretion, based on the type of bone screw, the surgeon may create a countersink.

A 3.5 mm compression screw is recommended but is not limited to that size.

If using a cannulated compression screw, a K-wire is fully inserted thru the axis hole to the plantar surface. The markings on a custom ROSE K-wire are examined to determine axis hole depth and appropriate compression screw length. A 3.5 mm, or other size cannulated compression screw is drilled through the axis hole, over the K-wire.

If using a non-cannulated and/or solid core compression screw, a K-wire is fully inserted thru the axis hole to the plantar surface. The markings on the custom ROSE K-wire are examined to determine axis hole depth and appropriate compression screw length, then removed. A 3.5 mm, or other size compression screw is drilled through the axis hole until fully seated. Alternatively, the length is determined by a standard measurement tool (e.g., hole depth gage) after the osteotomy is drilled.

Regardless of compression screw type (e.g., cannulated, or non-cannulated), a rotational burr is used to remove medial overhang prior to applying the dorsal or DP plate.

### ROSE Bunion Correction Procedure for a Fifth Metatarsal Bunion:

A ROSE Bunion Correction Procedure for a fifth metatarsal bunion deformity (hereinafter “Mini ROSE”), commonly referred to as Tailors Bunion deformity, is a bunion on the lateral aspect of a foot due to an increase in the intermetatarsal angle between the fourth and fifth metatarsals or increased distal bowing of the fifth metatarsal. The Mini ROSE technique is a proximal mid-shaft rotational osteotomy of the fifth metatarsal.

The ROSE Bunion Correction system and method utilizing a Mini ROSE technique may comprise an oblique osteotomy oriented from distal dorsal to proximal plantar executed on a fifth metatarsal from the lateral side. It is the same cut and rotation technique as on the medial bunion (e.g., ROSE Bunion Correction system and method) described herein. The same jig is used as in the first metatarsal technique except it is adapted for the fifth metatarsal. The osteotomy is fixated the same way with the independent compression screw and dorsal plate or dorsal plantar plate application in the same technique as in the first metatarsal ROSE Bunion Correction system and method described herein.

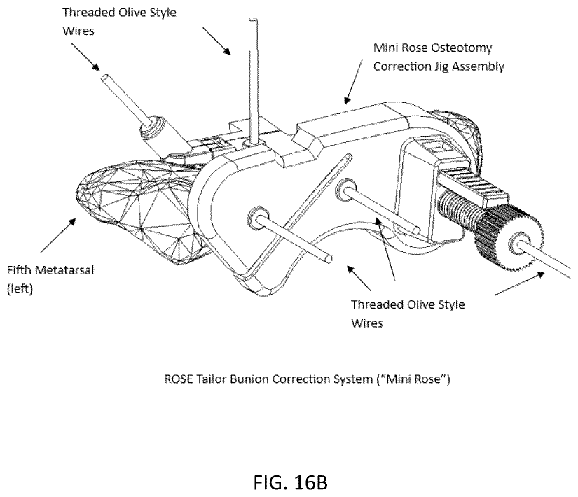

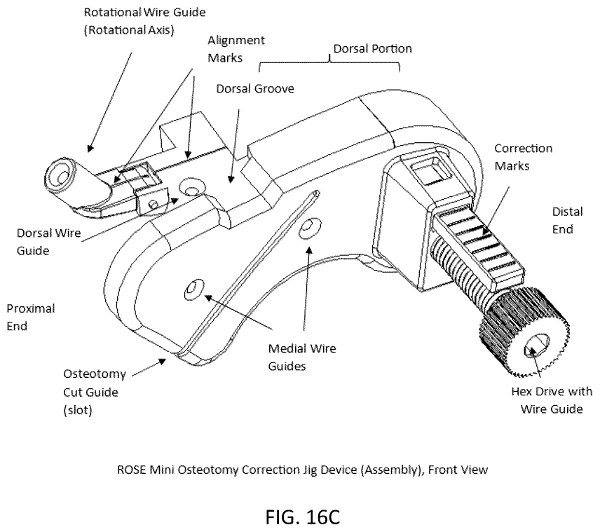

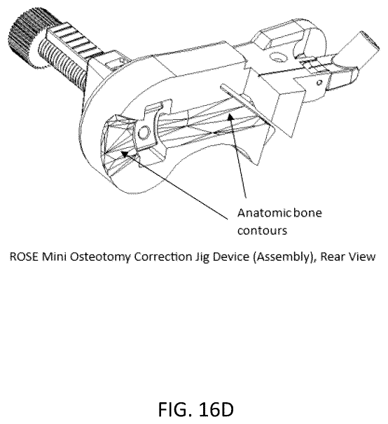

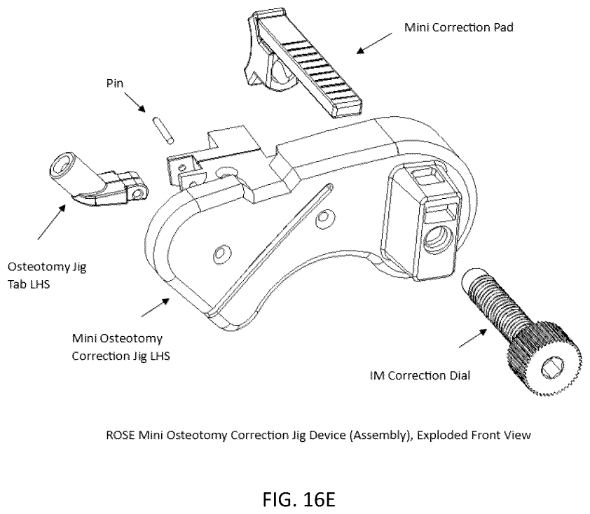

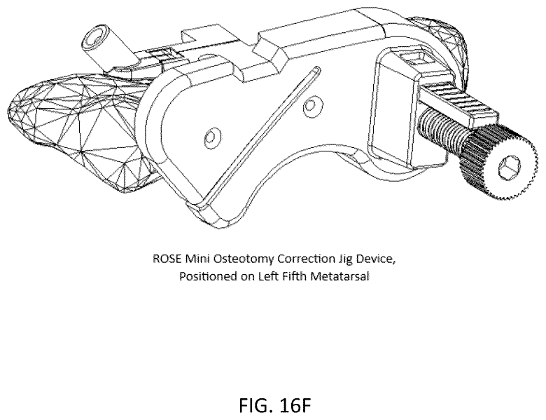

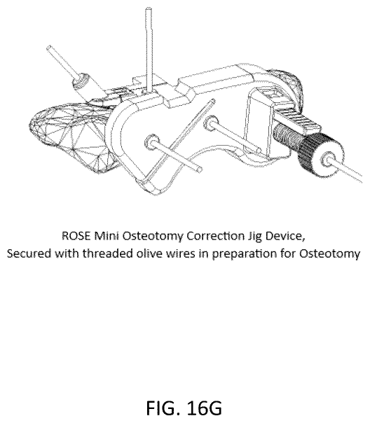

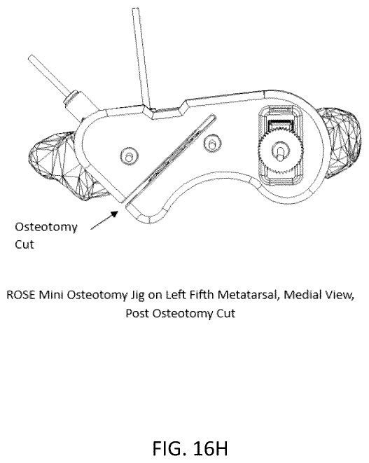

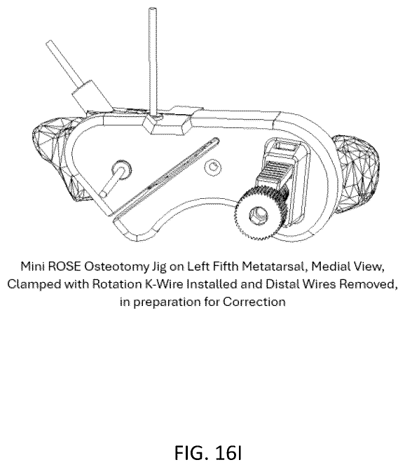

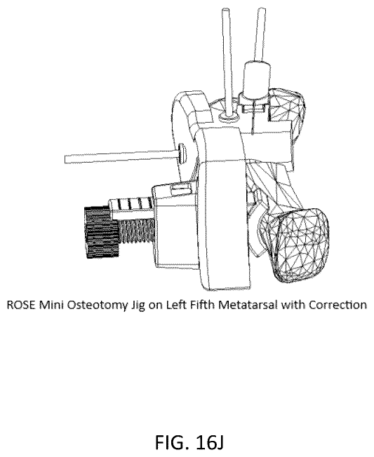

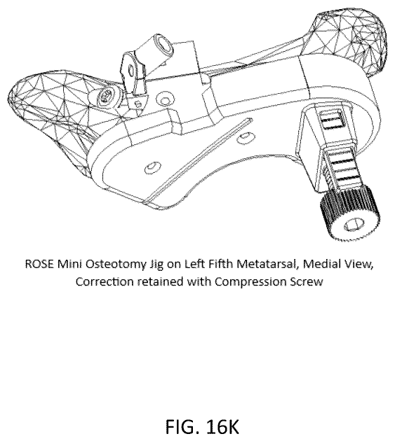

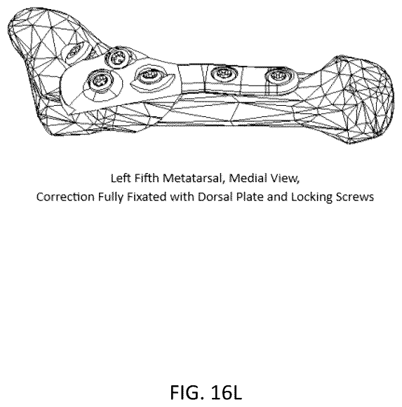

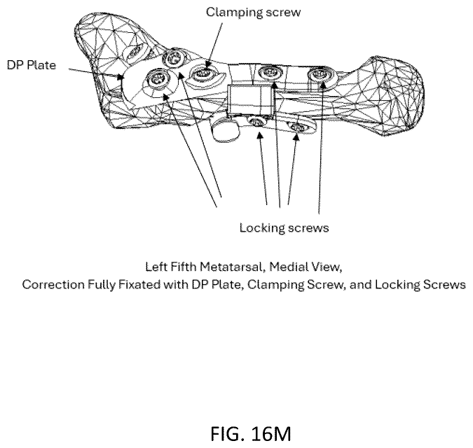

In FIG. 16A, a lateral view of a ROSE Bunion Correction system and method utilizing a Mini ROSE technique for a fifth metatarsal, Tailors bunion deformity oriented oblique osteotomy from dorsal distal to plantar proximal generally represented by reference numeral 160 and utilized in a ROSE bunionectomy procedure is shown, according to the present disclosure. The Mini ROSE technique 160 may comprise an orientation oblique osteotomy 162 from dorsal distal 163 to plantar proximal 164 of a fifth metatarsal 165 (e.g., left).

In FIG. 16B, a perspective view of a ROSE Bunion Correction system and method utilizing a Mini ROSE technique for a fifth metatarsal, Tailors bunion deformity oriented oblique osteotomy from dorsal distal to plantar proximal, positioned and secured on a fifth metatarsal with a Mini ROSE Osteotomy Correction Jig assembly generally represented by reference numeral 160 and utilized in a ROSE bunionectomy procedure is shown, according to the present disclosure. The Mini ROSE technique 160 may comprise an orientation oblique osteotomy 162 from dorsal distal 163 to plantar proximal 164 with a Mini ROSE Osteotomy Correction Jig assembly 161. The Mini ROSE Osteotomy Correction Jig assembly 161 may comprise a fifth metatarsal 165 (e.g., left), and threaded olive style wires 166 (or similar style wires).

In FIG. 16C, a front view of a Mini ROSE (a.k.a., ROSE Mini) Osteotomy Correction Jig assembly generally represented by reference numeral 161 and utilized in a ROSE bunionectomy procedure is shown, according to the present disclosure. The Mini ROSE Osteotomy Correction Jig assembly 161 may comprise an osteotomy cut guide (slot) 167, a dorsal wire guide 168, a rotational wire guide (rotational axis) 169, alignment marks 1600, a dorsal groove 1601, correction marks 1602, a hex drive with wire guide 1603, medial wire guides 1604, a proximal end 1605, and a distal end 1606.

In FIG. 16D, a rear view of a Mini ROSE (a.k.a., ROSE Mini) Osteotomy Correction Jig assembly generally represented by reference numeral 161 and utilized in a ROSE bunionectomy procedure is shown, according to the present disclosure. The Mini ROSE Osteotomy Correction Jig assembly 161 may comprise an osteotomy cut guide (slot) 167, a dorsal wire guide 168, a rotational wire guide (rotational axis) 169, alignment marks 1600, a dorsal groove 1601, correction marks 1602, a hex drive with wire guide 1603, medial wire guides 1604, a proximal end 1605, a distal end 1606, and anatomic bone contours 1607.

In FIG. 16E, an exploded, front view of a Mini ROSE (a.k.a., ROSE Mini) Osteotomy Correction Jig assembly generally represented by reference numeral 161 and utilized in a ROSE bunionectomy procedure is shown, according to the present disclosure. The Mini ROSE Osteotomy Correction Jig assembly 161 may comprise an osteotomy cut guide (slot) 167, a dorsal wire guide 168, a rotational wire guide (rotational axis) 169, alignment marks 1600, a dorsal groove 1601, correction marks 1602, a hex drive with wire guide 1603, medial wire guides 1604, a proximal end 1605, a distal end 1606, anatomic bone contours 1607 (not shown), a mini osteotomy correction jig LHS (e.g., left-hand side) 1608, an osteotomy jig tab LHS (e.g., left-hand side) 1609, a pin 1610, a mini correction pad 1611, and an IM correction dial 1612.

The novel ROSE Tailor Bunion Correction system (a.k.a., “Mini ROSE”), which includes features to execute the Osteotomy, correct the Intermetatarsal (IM) angle, and retain the IM angle correction without the need for removal of the Mini ROSE Osteotomy Correction Jig device/assembly from the metatarsal.

In other words, the Mini ROSE Osteotomy Correction Jig assembly is secured to the fifth metatarsal throughout the entire osteotomy and correction process, and partially during the fixation process. By retaining the device throughout most of the procedure (i.e., sans final fixation with bone plate), a more streamlined and repeatable procedure results, while minimizing the loss of desired correction in the time between correction and final fixation.

Similar in design and procedure steps as the ROSE Bunion Correction system and method, the novel ROSE Tailor Bunion Correction system (a.k.a. “Mini ROSE”) is intended for use with the fifth metatarsal.

### Additional Characteristics:

The Mini ROSE Osteotomy Correction Jig device may comprise a left-hand and right-hand version. The left-hand version is a true mirror of the right-hand version.

The Mini ROSE bunionectomy procedure utilizes additional instruments and implants already known in the arts including sagittal bone saws and blades, bone drills, Kirshner wires threaded and non-threaded, olive wires threaded and non-threaded, locking or non-locking bone screws, cannulated bone screws, compression screws, bone clamps including Verbrugge-style bone clamps, and similar.

### Mini ROSE Osteotomy Correction Jig Device

The Mini ROSE bunionectomy procedure provides benefits over other bunion correction protocols by (1) increasing or reducing the first intermetatarsal angle without a change in overall metatarsal length and (2) providing an optimized fixated fragment interface to assist in post ambulatory recovery.

These dual benefits are driven, in part, by the perpendicular relationship between the metatarsal dorsal and plantar fragment rotational axis and the osteotomy cut plane, which in turn becomes the contact interface between the fixated dorsal and plantar fragments. This optimized interface has a maximized surface area and is located on a plane that provides, once fixated, optimal support against typical post-ambulatory ground reaction force.

The Mini ROSE Osteotomy Correction Jig device was designed to readily determine proper positioning of the osteotomy cut and relative location of the rotational axis. The device may comprise positioning features geometrically designed to position with anatomic bone contours, features to accept and guide fixation wires, a slot feature to properly guide a sagittal saw blade fully through the metatarsal bone from a dorsal distal insertion to a plantar proximal exit without cartilage damage, and a manipulative guide feature to perpendicularly guide a Kirshner wire, or similar, towards and/or through the osteotomy cut that can be manipulated to provide clearance for a fixation screw, a correction assembly to relatively rotate the dorsal and plantar fragments to the desired intermetatarsal angle, and a measurement guide to aid the surgeon in the correction.

### Position

To utilize this device, the Mini ROSE Osteotomy Correction Jig device is first positioned using surgeon markings and/or anatomical features on the first metatarsal bone. This may comprise a mark applied by the surgeon to the mid-line dorsal surface of the exposed metatarsal bone prior to device placement, which is then aligned with markings on the dorsal portion of the device. This may further comprise surgeon marks to indicate the desired plantar exit of the osteotomy. Additionally, the device may comprise organically designed bosses and surfaces to contact and mate with typical bone surface geometry.

### Secure

Once properly positioned, the Mini ROSE Osteotomy Correction Jig device/assembly is secured to the metatarsal with a combination of dorsal and medial wires. The dorsal portion is secured by drilling a threaded olive wire through the rotational wire guide with the option for an additional threaded olive wire through the dorsal portion of the jig. Additionally, the device can be secured to the metatarsal bone in three medial locations by inserting threaded or unthreaded olive wires, or similar, through each of two wire location holes in the near face of the jig. The third wire is drilled through the center hole guide of the Intermetatarsal (IM) Correction Dial.

These fixation wires enter the device, pass through the device, and enter the medial face of the metatarsal. Countersinks on the top surface of the tab component and near face of the jig portion, at the wire location holes, are intended to mate with olive-style orthopedic wires, which is the preferred fixation wire type. Further, the device is mounted flush against the dorsal and medial side of the exposed first metatarsal.

### Cut

Correctly placed and secured, the jig clearly indicates and provides for proper positioning of the osteotomy cut. Further, the surgeon can execute the Mini ROSE osteotomy cut.

The jig portion of the Mini ROSE Osteotomy Correction Jig device may comprise an intuitive guide for the proper insertion of the sagittal saw blade at the distal dorsal end of the cut guide, controlled direction of the cut from the distal dorsal to plantar proximal end of the cut guide, and removal of the sagittal saw blade at the plantar proximal end of the cut guide. This provides for the proper execution of the Mini ROSE osteotomy cut, with respect to the anatomy of the first metatarsal, as well as the correction axis of rotation, controlled later in the procedure by the placement of the drilled rotational wire guide.

Once the osteotomy cut is successfully executed, the dorsal and plantar fragments are securely clamped in preparation for the full rotational K-wire insertion. This can be executed using a standard Verbrugge-style, or similar, bone clamp, based on the surgeon's discretion. The dorsal portion of the bone clamp is placed within the dorsal groove of the Mini ROSE Osteotomy Correction Jig device, with care to ensure the plantar portion will provide sufficient clearance to the plantar exit of the rotational K-wire (inserted in next procedure step).

### Correct

With the metatarsal dorsal and plantar fragments securely clamped, the rotational K-wire can be fully inserted through both the dorsal and plantar fragments.

First, the dorsal threaded olive wire is removed. This is replaced with a rotational K-wire guided through the same wire guide features of the Mini ROSE Osteotomy Correction Jig device tab, though fully drilled from the dorsal surface of the first metatarsal dorsal fragment through the plantar surface of the plantar fragment, perpendicularly crossing the osteotomy cut surfaces. In one embodiment of the Mini ROSE instrument set, this rotational K-wire is color coded, or etched, to provide a visual indication to the surgeon of the fully inserted K-wire insertion depth.

In preparation for the IM correction, the two distal olive wires are removed and the Verbrugge-style, or similar, bone clamp is released slightly. The dorsal and plantar fragments are now able to relatively rotate about the rotational K-wire, while maintaining a close planar positioning across the osteotomy cut.

By gripping the knurled end of the IM Correction Dial, or with a hex driver, the surgeon rotates the IM Correction Dial to execute the desired correction. The Mini Correction Pad, driven by the interface between the Correction Dial and internal threads of the jig portion, engages and moves the distal portion of the plantar fragment away from the jig face, and rotates the fragment around the K-wire to the desired IM angle position. If needed, the surgeon can rotate the IM Correction Dial in the opposite direction to reduce or adjust the correction.

Once the desired correction is achieved, the Verbrugge-style, or similar, clamp is once again firmly clamped, in preparation for initial fixation.

### Retain

To retain the IM correction, the surgeon first removes the rotational K-wire from the dorsal rotational wire hole of the tab feature. This tab feature is then flipped upward away from the metatarsal distal surface. This provides clearance for locking or non-locking screw insertion. At the surgeon's discretion, and based on the type of bone screw, the surgeon may create a countersink.

A 2.0 mm or 2.7 mm compression screw is recommended but is not limited to that size.

If using a cannulated locking or non-locking screw, a K-wire is fully inserted thru the axis hole to the plantar surface. The markings on the custom Mini ROSE K-wire are examined to determine axis hole depth and appropriate locking screw length. A compression screw is drilled through the axis hole, over the K-wire.

If using a non-cannulated locking or non-locking screw, a K-wire is fully inserted thru the axis hole to the plantar surface. The markings on the custom Mini ROSE K-wire are examined to determine axis hole depth and appropriate locking or non-locking screw length, then removed. Alternatively, the length is determined by a standard measurement tool (e.g., hole depth gage) after the osteotomy is drilled. A compression screw is drilled through the axis hole until fully seated.

Regardless of locking screw type (e.g., cannulated, or non-cannulated), a rotational burr is used to remove medial overhang prior to applying the dorsal or DP plate.

### Fixation

Fixation is similar to the ROSE bunionectomy procedure, with an initial compression screw for compression and retention of correction and dorsal plate with locking or non-locking screws, or an initial compression screw and dorsal plantar (DP) plate with clamping and locking or non-locking screws. The osteotomy can be rotated or translated.

In FIG. 16F, a perspective view of a ROSE Bunion Correction system and method utilizing a Mini ROSE technique for a fifth metatarsal, Tailors bunion deformity oriented oblique osteotomy from dorsal distal to plantar proximal, positioned on the left fifth metatarsal with a Mini ROSE (a.k.a., ROSE Mini) Osteotomy Correction Jig assembly generally represented by reference numeral 160 and utilized in a ROSE bunionectomy procedure is shown, according to the present disclosure. The Mini ROSE technique 160 may comprise an orientation oblique osteotomy 162 from dorsal distal 163 to plantar proximal 164 with a Mini ROSE Osteotomy Correction Jig assembly 161. The Mini ROSE Osteotomy Correction Jig assembly 161 may comprise a fifth metatarsal 165 (e.g., left), an osteotomy cut guide (slot) 167, a dorsal wire guide 168, a rotational wire guide (rotational axis) 169, alignment marks 1600, a dorsal groove 1601, correction marks 1602, a hex drive with wire guide 1603, medial wire guides 1604, a proximal end 1605, a distal end 1606, anatomic bone contours 1607 (not shown), a mini osteotomy correction jig LHS (e.g., left-hand side) 1608, an osteotomy jig tab LHS (e.g., left-hand side) 1609, a pin 1610, a mini correction pad 1611, and an IM correction dial 1612.

In FIG. 16G, a perspective view of a ROSE Bunion Correction system and method utilizing a Mini ROSE technique for a fifth metatarsal, Tailors bunion deformity oriented oblique osteotomy from dorsal distal to plantar proximal, positioned and secured by threaded olive wires on a fifth metatarsal with a Mini ROSE (a.k.a., ROSE Mini) Osteotomy Correction Jig assembly in preparation for an Osteotomy generally represented by reference numeral 160 and utilized in a ROSE bunionectomy procedure is shown, according to the present disclosure. The Mini ROSE technique 160 may comprise an orientation oblique osteotomy 162 from dorsal distal 163 to plantar proximal 164 with a Mini ROSE Osteotomy Correction Jig assembly 161. The Mini ROSE Osteotomy Correction Jig assembly 161 may comprise a fifth metatarsal 165 (e.g., left), threaded olive style wires 166 (or similar style wires), an osteotomy cut guide (slot) 167, a dorsal wire guide 168, a rotational wire guide (rotational axis) 169, alignment marks 1600, a dorsal groove 1601, correction marks 1602, a hex drive with wire guide 1603, medial wire guides 1604, a proximal end 1605, a distal end 1606, anatomic bone contours 1607 (not shown), a mini osteotomy correction jig LHS (e.g., left-hand side) 1608, an osteotomy jig tab LHS (e.g., left-hand side) 1609, a pin 1610, a mini correction pad 1611, and an IM correction dial 1612.

In FIG. 16H, a medial view of a ROSE Bunion Correction system and method utilizing a Mini ROSE technique for a fifth metatarsal, Tailors bunion deformity oriented oblique osteotomy from dorsal distal to plantar proximal, positioned and secured by threaded olive wires on the left fifth metatarsal, and after the Osteotomy with a Mini ROSE (a.k.a., ROSE Mini) Osteotomy Correction Jig assembly generally represented by reference numeral 160 and utilized in a ROSE bunionectomy procedure is shown, according to the present disclosure. The Mini ROSE technique 160 may comprise an orientation oblique osteotomy 162 from dorsal distal 163 to plantar proximal 164 with a Mini ROSE Osteotomy Correction Jig assembly 161. The Mini ROSE Osteotomy Correction Jig assembly 161 may comprise a fifth metatarsal 165 (e.g., left), threaded olive style wires 166 (or similar style wires), an osteotomy cut guide (slot) 167, a dorsal wire guide 168, a rotational wire guide (rotational axis) 169, alignment marks 1600, a dorsal groove 1601, correction marks 1602, a hex drive with wire guide 1603, medial wire guides 1604, a proximal end 1605, a distal end 1606, anatomic bone contours 1607 (not shown), a mini osteotomy correction jig LHS (e.g., left-hand side) 1608, an osteotomy jig tab LHS (e.g., left-hand side) 1609, a pin 1610, a mini correction pad 1611, an IM correction dial 1612, an osteotomy cut 1613, a dorsal metatarsal fragment 1614, and a plantar metatarsal fragment 1615.

In FIG. 16I, a medial view of a ROSE Bunion Correction system and method utilizing a Mini ROSE technique for a fifth metatarsal, Tailors bunion deformity oriented oblique osteotomy from dorsal distal to plantar proximal, after the Osteotomy, and clamped with a rotation K-wire installed and distal wires removed, in preparation for bone correction with a Mini ROSE (a.k.a., ROSE Mini) Osteotomy Correction Jig assembly generally represented by reference numeral 160 and utilized in a ROSE bunionectomy procedure is shown, according to the present disclosure. The Mini ROSE technique 160 may comprise an orientation oblique osteotomy 162 from dorsal distal 163 to plantar proximal 164 with a Mini ROSE Osteotomy Correction Jig assembly 161. The Mini ROSE Osteotomy Correction Jig assembly 161 may comprise a fifth metatarsal 165 (e.g., left), threaded olive style wires 166 (or similar style wires), an osteotomy cut guide (slot) 167, a dorsal wire guide 168, a rotational wire guide (rotational axis) 169, alignment marks 1600, a dorsal groove 1601, correction marks 1602, a hex drive with wire guide 1603, medial wire guides 1604, a proximal end 1605, a distal end 1606, anatomic bone contours 1607 (not shown), a mini osteotomy correction jig LHS (e.g., left-hand side) 1608, an osteotomy jig tab LHS (e.g., left-hand side) 1609, a pin 1610, a mini correction pad 1611, an IM correction dial 1612, an osteotomy cut 1613, a dorsal metatarsal fragment 1614, a plantar metatarsal fragment 1615, and a rotational K-wire 1616.

In FIG. 16J, a right side, perspective view of a ROSE Bunion Correction system and method utilizing a Mini ROSE technique for a fifth metatarsal, Tailors bunion deformity oriented oblique osteotomy from dorsal distal to plantar proximal, after the Osteotomy, clamped with a rotation K-wire installed and distal wires removed, with bone correction, and with a Mini ROSE (a.k.a., ROSE Mini) Osteotomy Correction Jig assembly generally represented by reference numeral 160 and utilized in a ROSE bunionectomy procedure is shown, according to the present disclosure. The Mini ROSE technique 160 may comprise an orientation oblique osteotomy 162 from dorsal distal 163 to plantar proximal 164 with a Mini ROSE Osteotomy Correction Jig assembly 161. The Mini ROSE Osteotomy Correction Jig assembly 161 may comprise a fifth metatarsal 165 (e.g., left), threaded olive style wires 166 (or similar style wires), an osteotomy cut guide (slot) 167, a dorsal wire guide 168, a rotational wire guide (rotational axis) 169, alignment marks 1600, a dorsal groove 1601, correction marks 1602, a hex drive with wire guide 1603, medial wire guides 1604, a proximal end 1605, a distal end 1606, anatomic bone contours 1607 (not shown), a mini osteotomy correction jig LHS (e.g., left-hand side) 1608, an osteotomy jig tab LHS (e.g., left-hand side) 1609, a pin 1610, a mini correction pad 1611, an IM correction dial 1612, an osteotomy cut 1613, a dorsal metatarsal fragment 1614, a plantar metatarsal fragment 1615, and a rotational K-wire 1616.

In FIG. 16K, a medial view of a ROSE Bunion Correction system and method utilizing a Mini ROSE technique for a fifth metatarsal, Tailors bunion deformity oriented oblique osteotomy from dorsal distal to plantar proximal, after the Osteotomy, with wires removed, with bone correction retained with a compression screw, and with a Mini ROSE (a.k.a., ROSE Mini) Osteotomy Correction Jig assembly generally represented by reference numeral 160 and utilized in a ROSE bunionectomy procedure is shown, according to the present disclosure. The Mini ROSE technique 160 may comprise an orientation oblique osteotomy 162 from dorsal distal 163 to plantar proximal 164 with a Mini ROSE Osteotomy Correction Jig assembly 161. The Mini ROSE Osteotomy Correction Jig assembly 161 may comprise a fifth metatarsal 165 (e.g., left), threaded olive style wires 166 (or similar style wires), an osteotomy cut guide (slot) 167, a dorsal wire guide 168, a rotational wire guide (rotational axis) 169, alignment marks 1600, a dorsal groove 1601, correction marks 1602, a hex drive with wire guide 1603, medial wire guides 1604, a proximal end 1605, a distal end 1606, anatomic bone contours 1607 (not shown), a mini osteotomy correction jig LHS (e.g., left-hand side) 1608, an osteotomy jig tab LHS (e.g., left-hand side) 1609, a pin 1610, a mini correction pad 1611, an IM correction dial 1612, an osteotomy cut 1613, a dorsal metatarsal fragment 1614, a plantar metatarsal fragment 1615, and a compression screw 1617.

In FIG. 16L, a medial view of a ROSE Bunion Correction system and method utilizing a Mini ROSE technique for a fifth metatarsal, Tailors bunion deformity oriented oblique osteotomy from dorsal distal to plantar proximal, after the Osteotomy, with bone correction fully fixated with a dorsal plate and locking or non-locking screws generally represented by reference numeral 160 and utilized in a ROSE bunionectomy procedure is shown, according to the present disclosure. The Mini ROSE technique 160 may comprise an orientation oblique osteotomy 162 from dorsal distal 163 to plantar proximal 164. The Mini ROSE technique 160 may further comprise a dorsal bone plate 1618, and locking or non-locking screws 1619.

In FIG. 16M, a medial view of a ROSE Bunion Correction system and method utilizing a Mini ROSE technique for a fifth metatarsal, Tailors bunion deformity oriented oblique osteotomy from dorsal distal to plantar proximal, after the Osteotomy, with bone correction fully fixated with a dorsal plantar (DP) plate, clamping screw and locking or non-locking screws generally represented by reference numeral 160 and utilized in a ROSE bunionectomy procedure is shown, according to the present disclosure. The Mini ROSE technique 160 may comprise an orientation oblique osteotomy 162 from dorsal distal 163 to plantar proximal 164. The Mini ROSE technique 160 may further comprise a dorsal plantar (DP) bone plate 1620, locking or non-locking screws 1619, and a clamping screw 1621.

### ROSE Bunion Correction Procedure for a Ludloff Osteotomy:

A ROSE Bunion Correction system and method utilizing a Ludloff osteotomy (hereinafter “ROSE D”) may comprise an oblique osteotomy for the correction of hallux valgus, bunion deformities. The ROSE D technique is oriented from dorsal proximal to distal plantar. The osteotomy is made with the same jig, specifically oriented for the Ludloff cut, in the same technique and fixation with the independent compression screw and then dorsal or dorsal plantar plate as with the ROSE Bunion Correction system and method described herein. The osteotomy can be rotated or translated. A dorsal flexing or plantar flexing wedge osteotomy may be fixated by the dorsal plantar (DP) or dorsal bone plate.

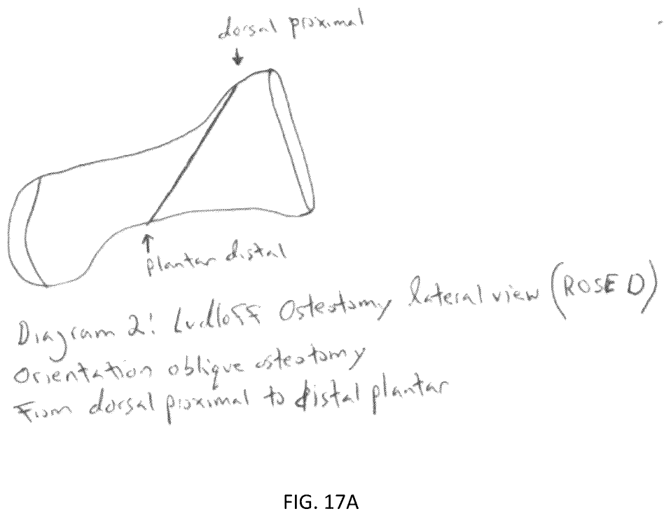

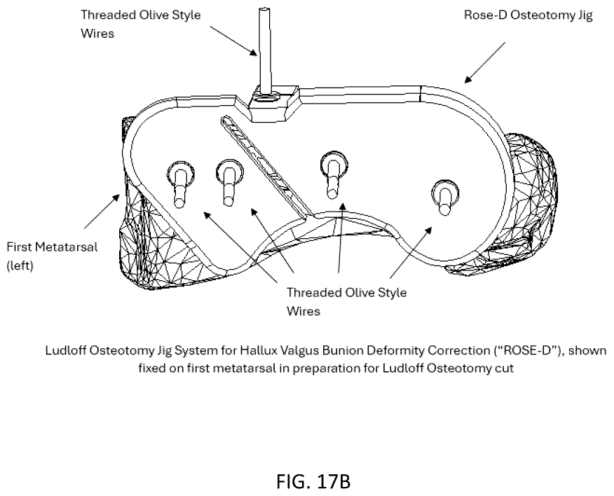

In FIG. 17A, a lateral view of a ROSE Bunion Correction system and method utilizing a ROSE D technique, Ludloff Osteotomy oriented oblique osteotomy from dorsal proximal to plantar distal generally represented by reference numeral 170 and utilized in a ROSE bunionectomy procedure is shown, according to the present disclosure. The ROSE D technique 170 may comprise an orientation oblique osteotomy 172 from dorsal proximal 173 to plantar distal 174 of a first metatarsal 175 (e.g., left).

### Ludloff Osteotomy Correction Jig for Hallux Valgus Osteotomy Correction (“ROSE-D”)

The Ludloff Osteotomy Correction Jig (hereinafter “ROSE-D Osteotomy Correction Jig”) was designed to readily determine proper positioning of the Ludloff osteotomy cut for Hallux Valgus correction. The device may comprise positioning features geometrically designed to position with anatomic bone contours, features to accept and guide fixation wires, and a slot feature to properly guide a sagittal saw blade fully through the metatarsal bone from a dorsal proximal insertion to a plantar distal exit without cartilage damage. The ROSE-D technique is a proximal mid-shaft rotational and/or translational osteotomy.

### Position

To utilize this device, the ROSE-D Osteotomy Correction Jig is first positioned using surgeon markings and/or anatomical features on the first metatarsal bone. This may comprise a mark applied by the surgeon to the mid-line dorsal surface of the exposed metatarsal bone prior to device placement, which is then aligned with markings on the dorsal portion of the device. This may further comprise surgeon marks to indicate the desired plantar exit of the osteotomy. Additionally, the device may comprise organically designed bosses and surfaces to contact and mate with typical bone surface geometry.

### Secure

Once properly positioned, the ROSE-D Osteotomy Correction Jig is secured to the metatarsal with a combination of dorsal and medial wires. The dorsal portion is secured by drilling a threaded olive wire through the dorsal wire guide. Additionally, the device can be secured to the metatarsal bone in four medial locations by inserting threaded or unthreaded olive wires, or similar, through each of four wire location holes in the near face of the jig.

These fixation wires enter the device, pass through the device, and enter the medial face of the metatarsal. Countersinks on the dorsal surface of the jig and near the medial face of the jig portion, at the wire location holes, are intended to mate with olive-style orthopedic wires, which is the preferred fixation wire type. Further, the device is mounted flush against the dorsal and medial side of the exposed first metatarsal.

### Cut

Correctly placed and secured, the jig clearly indicates and provides for proper positioning of the osteotomy cut. Further, the surgeon can execute the ROSE-D osteotomy cut.

The ROSE-D Osteotomy Correction Jig may comprise an intuitive guide for the proper insertion at the dorsal proximal end of the cut guide, controlled direction of the cut from the dorsal proximal to plantar distal end of the cut guide, and removal of the sagittal saw blade at the plantar distal end of the cut guide. This provides for the proper execution of the ROSE-D osteotomy cut.

### Fixation

Fixation is similar to the ROSE bunionectomy procedure with an initial compression screw and dorsal plate with locking or non-locking screws, or an initial compression screw and dorsal plantar (DP) plate with clamping and locking or non-locking screws. The osteotomy can be rotated or translated.

In FIG. 17B, a lateral view of a ROSE Bunion Correction system and method utilizing a ROSE D technique, Ludloff Osteotomy oriented oblique osteotomy from dorsal proximal to plantar distal positioned and secured on a first metatarsal with a ROSE-D (a.k.a., Ludloff) Osteotomy Correction Jig assembly/system in preparation for a Ludloff Osteotomy generally represented by reference numeral 170 and utilized in a ROSE bunionectomy procedure is shown, according to the present disclosure. The ROSE D technique 170 may comprise an orientation oblique osteotomy 172 from dorsal proximal 173 to plantar distal 174 with a ROSE-D Osteotomy Correction Jig assembly 171. The ROSE-D Osteotomy Correction Jig assembly 171 may comprise a first metatarsal 175 (e.g., left), and threaded olive style wires 176 (or similar style wires).

Osteotomy Guide Clamp with Folding Handles

In one embodiment, the ROSE Bunion Correction system and method may comprise a unique “hinge clamp,” which is attached to the rotational osteotomy cut jig. The hinged clamp is needed to allow the handles and body of the clamp to be moved out of the way so that they do not interfere with the operating of the saw as it is inserted in the jig and cuts the bone.

This unique apparatus, which has applications in surgical specialties of all disciplines, may comprise a hinge built into the clamp to enable the handles and body of the clamp to be repositioned in different directions to allow freedom from an intrinsic block, which would otherwise be caused by the handles and body of the clamp.

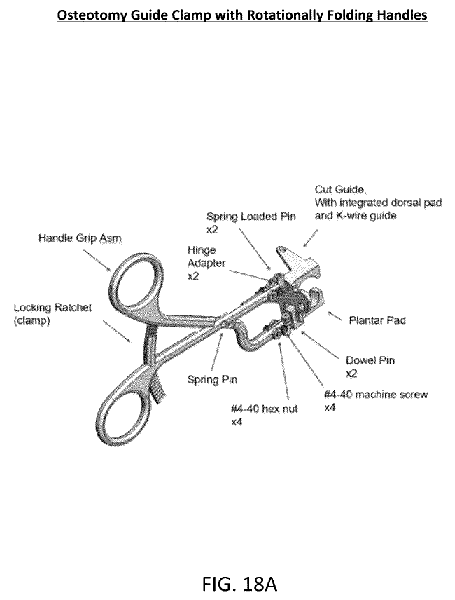

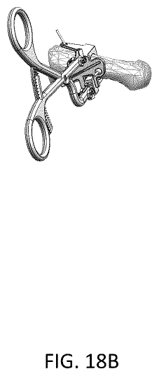

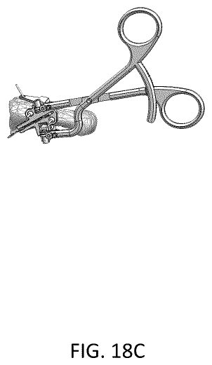

In FIG. 18A, a right side, perspective view of an Osteotomy Guide Clamp with rotationally folding handles generally represented by reference numeral 180 and utilized in a ROSE bunionectomy procedure is shown, according to the present disclosure. The Osteotomy Guide Clamp 180 may comprise a locking ratchet (clamp) 181, a handle grip Asm 182, hinge adapters 183, spring loaded pins 184, a cut guide, with integrated dorsal pad and K-wire guide 185, a plantar pad 186, dowel pins 187, machine screws 188, hex nuts 189, and a spring pin 1800.

The unique system and method described herein utilizes this novel clamp design with jig apparatus and rotational folding clamp handles for consistent reliable reproduction of the osteotomy cut.

### Securing the Osteotomy Guide Clamp on the Exposed First Metatarsal:

The upper portion of the clamp is positioned over the previously located and partially drilled guide wire, until the dorsal pad contacts the dorsal portion of the exposed metatarsal.

In FIG. 18B, a right side, perspective view of an Osteotomy Guide Clamp with rotationally folding handles with jig feature mounted perpendicular to the lateral plane of the exposed first metatarsal generally represented by reference numeral 180 and utilized in a ROSE bunionectomy procedure is shown, according to the present disclosure. The Osteotomy Guide Clamp 180 with jig feature 1801 may comprise a locking ratchet (clamp) 181, a handle grip Asm 182, hinge adapters 183, spring loaded pins 184, a cut guide, with integrated dorsal pad and K-wire guide 185, a plantar pad 186, dowel pins 187, machine screws 188, hex nuts 189, a spring pin 1800, and a metatarsal 1802.

The jig apparatus may include two laterally positioned holes to accept additional positioning wires, such as a K-wire style with ball feature (e.g., Stryker BB-TAK). K-wires with a ball feature are inserted through each hole. The jig feature is mounted flush against the lateral side of the exposed first metatarsal.

Once the upper portion is properly positioned, the device is securely clamped around the metatarsal by squeezing the handles until the lower portion of the clamp engages the plantar surface of the metatarsal. Ratcheting features on the handles retain the secure clamping.

Alternatively, the upper and lower portions of the clamp can be spaced open, then placed over and around the metatarsal. The device is securely clamped around the metatarsal by squeezing the handles until the upper portion of the clamp (i.e., dorsal pad) securely engages the dorsal surface of the metatarsal and the lower portion of the clamp (i.e., plantar pad) securely engages the plantar surface of the metatarsal. The pads are designed to conform to specific anatomical markers of the metatarsal, allowing for the proper placement of the device.

Ratcheting features on the handles retain the secure clamping. The jig feature may comprise two laterally positioned holes to accept additional positioning wires, such as a K-wire style with ball feature. When the K-wires with ball feature are inserted, the jig feature is mounted flush against the lateral side of the exposed first metatarsal.

Once the device is securely clamped on the metatarsal, the rotational guide K-wire is drilled from the dorsal surface, approximately ⅓ into the dorsal cortex.

### Preparing for the Osteotomy Cut

The jig feature of the device provides an intuitive guide for the osteotomy cut, and ensures the cut is perpendicular to the axis of the rotation guide K-wire. Maintaining this perpendicular relationship maximizes the bearing surface of the corrected bunion without changing the overall length of the metatarsal.

The device may comprise a hinge design that connects the lower handle to the dorsal pad portion of the upper clamp (upper hinge). A similar hinge design connects the upper handle to the plantar pad portion of the lower clamp (lower hinge).

To maximize clamp force, the axis of the upper hinge is collinear to the axis of the lower hinge.

The device handles can be rotated in unison with the hinge axis as the center of rotation. Detents within the hinge assembly, including but not limited to spring assist, allow the device handles to be retained in set angular positions.

To clamp the device, the handles are set in a position essentially perpendicular to the lateral plane of the metatarsal. When the device handles are squeezed together, the hinges transmit the applied force to clamp the dorsal and plantar pads securely against the metatarsal. The pads are designed to conform to specific anatomical markers of the metatarsal, further reinforcing the proper placement of the device.

In FIG. 18C, a left side, perspective view of an Osteotomy Guide Clamp with rotationally folding handles with jig feature mounted perpendicular to the lateral plane of an exposed first metatarsal with handles rotationally folded towards the distal end of the metatarsal and retained by the distal detent position generally represented by reference numeral 180 and utilized in a ROSE bunionectomy procedure is shown, according to the present disclosure. The Osteotomy Guide Clamp 180 with jig feature 1801 may comprise a locking ratchet (clamp) 181, a handle grip Asm 182, hinge adapters 183, spring loaded pins 184, a cut guide, with integrated dorsal pad and K-wire guide 185, a plantar pad 186, dowel pins 187, machine screws 188, hex nuts 189, a spring pin 1800, and a metatarsal 1802.

When in the clamping position, the handles prevent full visual exposure of the osteotomy guide slot for the surgeon and physical interference with the sagittal saw. To remedy this, the handles can be rotationally folded towards the distal end of the metatarsal and retained by the distal detent position.

### Securing Fragments for Correction Preparation Without Device Removal

The plantar/dorsal pad designs and upper/lower clamp orientations allow for re-clamping of the dorsal and plantar metatarsal fragments without the need for device removal. To secure the fragments, the device handles are rotated to a perpendicular position, as determined by the detent position, and squeeze the handles. The ratcheting features of the handles hold the clamp in position.

### Additional Benefits of Rotational Handles

The ROSE Bunion Correction system and method aids the surgeon with a set of clamps, plate and procedure options. One such procedure option is to retain the Osteotomy Guide Clamp in the proper position while placing the Reduction Clamp in the proper position and executing the correction. This can occur by rotationally folding the handles of the Osteotomy Guide Clamp in the proximal position, which prevents any interference with the Reduction Clamp.

Additional Surgical Clamps with Folding Handles

The rotational folding handles of the Osteotomy Guide Clamp can be readily applied to other clamps within the ROSE Bunion Correction system and method, such as the Reverdin Clamp. For the Reverdin Clamp, as with the Osteotomy Guide Clamp, the folding handles allow the surgeon to apply the clamping force required during device placement, and once rotationally folded distally (or proximally if appropriate), provides the physical clearance necessary to provide the surgeon with a maximized field of view and prevent instrument interference (e.g., sagittal saw and clamp handles).

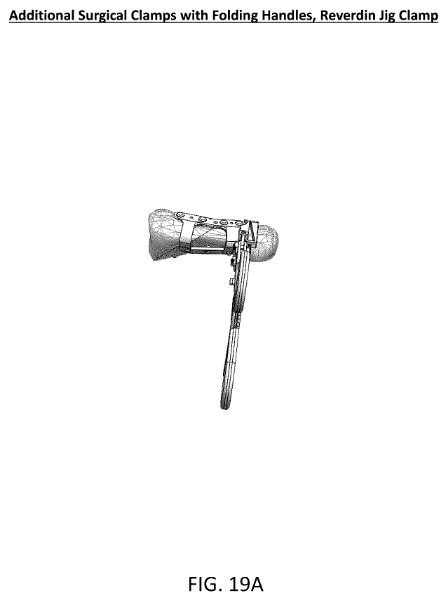

In FIG. 19A, a front side, perspective view of a Reverdin Clamp with rotationally folding handles with jig feature mounted perpendicular to the lateral plane of the head of an exposed first metatarsal generally represented by reference numeral 190 and utilized in a ROSE bunionectomy procedure is shown, according to the present disclosure. The Reverdin Clamp 190 with jig feature 191 may comprise rotationally folding handles 192, and a metatarsal 193.

In FIG. 19B, a right side, perspective view of a Reverdin Clamp with rotationally folding handles with jig feature mounted perpendicular to the lateral plane of the head of an exposed first metatarsal with handles rotationally folded towards the proximal end of the metatarsal and retained by the proximal detent position generally represented by reference numeral 190 and utilized in a ROSE bunionectomy procedure is shown, according to the present disclosure. The Reverdin Clamp 190 with jig feature 191 may comprise rotationally folding handles 192, and a metatarsal 193.

Additionally, the rotational folding handles of the Osteotomy Guide Clamp can be readily applied to other clamps outside the ROSE Bunion Correction system and method, such as the Verbrugge style or other standard bone clamps.

### Curved Bone Plate

In one embodiment, the ROSE Bunion Correction system and method may comprise a unique curved bone plate, i.e., lobster plate-it is a locking plate (e.g., meaning the screws are locked into the plate from the dorsal and plantar surfaces). This curved bone plate design allows for maximal compression from the dorsal to plantar surface perpendicular to the cut, and also from the plantar to dorsal surface perpendicular to the cut. This enables a clamp like closure of the osteotomy.

Currently, bunionectomy fixations are performed using straight bone plates or plates with some, though limited, curvature that follows the anatomical markers of the first metatarsal.

The ROSE Bunion Correction system and method may comprise the option of a curved bone plate that securely fixates the dorsal and plantar fragments. The novel curved bone plate extends further around the corrected metatarsal than current devices. The design of the plate allows the surgeon to fixate some bone screws in a perpendicular orientation to the osteotomy cut for improved, more efficient compression and an expectation for improved force (weight) bearing over the osteotomy cut surfaces.

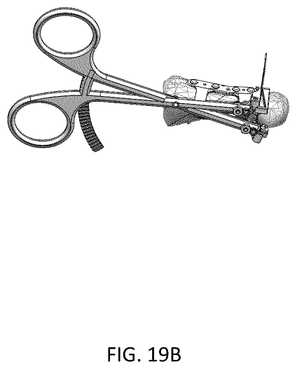

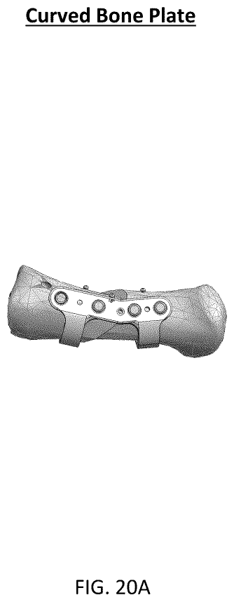

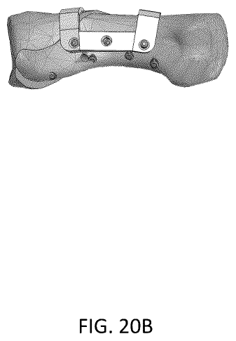

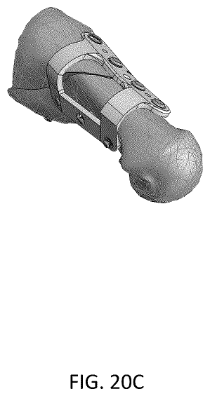

In FIG. 20A, a dorsal side, perspective view of a curved bone plate that securely fixates the dorsal and plantar fragments of an exposed first metatarsal generally represented by reference numeral 200 and utilized in a ROSE bunionectomy procedure is shown, according to the present disclosure. The curved bone plate 200 may comprise a dorsal metatarsal fragment 201, a plantar metatarsal fragment 201, and locking or non-locking screw clearance holes 202.

In FIG. 20B, a plantar side, perspective view of a curved bone plate that securely fixates the dorsal and plantar fragments of an exposed first metatarsal generally represented by reference numeral 200 and utilized in a ROSE bunionectomy procedure is shown, according to the present disclosure. The curved bone plate 200 may comprise a dorsal metatarsal fragment 201, a plantar metatarsal fragment 201, locking or non-locking screw clearance holes 202, locking or non-locking screws 203, and a clamping screw 204.

In FIG. 20C, a right side, perspective view of a curved bone that securely fixates the dorsal and plantar fragments of an exposed first metatarsal generally represented by reference numeral 200 and utilized in a ROSE bunionectomy procedure is shown, according to the present disclosure. The curved bone plate 200 may comprise a dorsal metatarsal fragment 201, a plantar metatarsal fragment 201, locking or non-locking screw clearance holes 202, locking or non-locking screws 203, and a clamping screw 204.

The curved bone plate features a dorsal portion, a plantar portion, and two connection webs. In an alternative embodiment, the curved bone plate replaces the two connection webs with one central web.

### Correction Clamp

In one embodiment, the ROSE Bunion Correction system and method may comprise a unique correction clamp that can rotate the first metatarsal bone around an axis pin to reduce or decrease the first intermetatarsal angle.

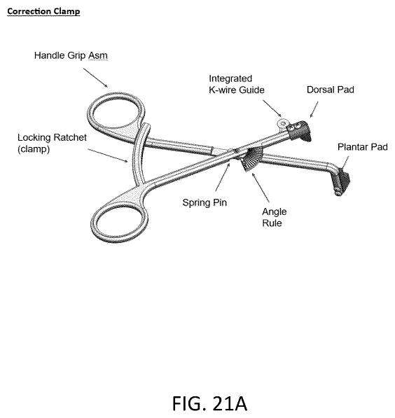

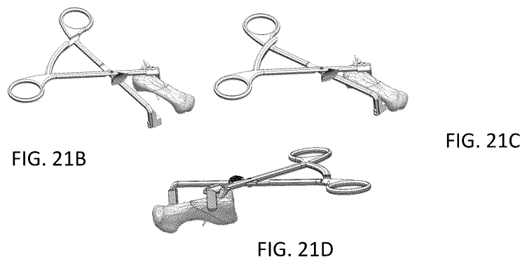

In FIG. 21A, a right side, perspective view of a correction clamp that rotates the first metatarsal bone around an axis pin to reduce or decrease the first intermetatarsal angle generally represented by reference numeral 210 and utilized in a ROSE bunionectomy procedure is shown, according to the present disclosure. The correction clamp 210 may comprise a locking ratchet (clamp) 211, a handle grip Asm 212, an integrated K-wire guide 213, a dorsal pad 214, a plantar pad 215, an angle rule 216, and a spring pin 217.

A key benefit of the ROSE bunionectomy procedure is the perpendicular relationship between the correction (i.e., reduction) axis and the osteotomy cut plane. With this relationship, the bunion correction maximizes the fragment bearing surfaces without a change in the overall length of the metatarsal.

To ensure the angular correction occurs around the optimal axis of rotation, the novel correction clamp may comprise an integrated K-wire guide. The correction clamp (also known as reduction clamp), is positioned over and down along the previously placed rotational K-wire guide while the dorsal pad is guided along the medial surface of the metatarsal. The dorsal pad has a minimal geometry to contact the surface of the dorsal metatarsal fragment correctly in the relatively small available metatarsal surface space and incision clearance. The correction clamp is fully guided down along the K-wire until the handle surface securely contacts the plantar surface of the metatarsal. This portion of the clamp now works as the fulcrum of the angular reduction device.

In FIGS. 21B, 21C, and 21D, perspective views of a correction clamp rotating the first metatarsal bone around an axis pin to reduce or decrease the first intermetatarsal angle generally represented by reference numeral 210 and utilized in a ROSE bunionectomy procedure are shown, according to the present disclosure. The correction clamp 210 may comprise a locking ratchet (clamp) 211, a handle grip Asm 212, an integrated K-wire guide 213, a dorsal pad 214, a plantar pad 215, an angle rule 216, and a spring pin 217.

The clamp may comprise an angle guide to aid the surgeon with the correct amount of angular reduction. The clamp handles are squeezed until the plantar pad contacts the lateral surface of the plantar metatarsal fragment. As the handles are further squeezed to provide the relative rotation of the dorsal and plantar fragments around the rotational axis, and during this motion, the surgeon can reference the angle rule as a guide for the level of angle correction.

The correction can be held in position using the locking ratchet features of the handles while the fragments are held in place with a standard bone clamp in preparation for replacement of the K-wire guide with a cannulated locking or non-locking screw.

### ROSE Bunion Correction Extended Plates

In an alternative embodiment of the Dorsal bone plate (hereinafter “Dorsal plate”) and an alternative embodiment of the Dorsal Plantar (DP) bone plate (hereinafter “Dorsal Plantar (DP) plate”), these plates may comprise an extended geometry in the proximal dorsal area according to the present disclosure.

In these embodiments described herein, the plates (e.g., dorsal bone plate and/or dorsal plantar bone plate) may comprise an extended geometry in the proximal dorsal area. The independent compression screw of the first embodiment is captured within the plate. The position and function of the compression screw does not change as described herein.

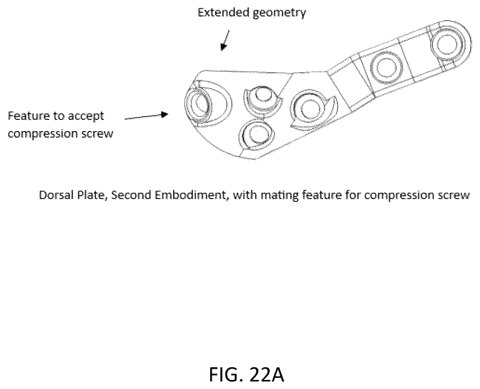

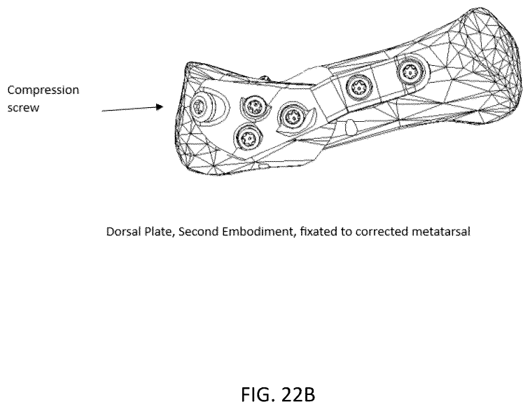

In FIG. 22A, a perspective view of an alternative embodiment of a dorsal bone plate with mating feature for compression screw generally represented by reference numeral 220 and utilized in a ROSE bunionectomy procedure is shown, according to the present disclosure. The dorsal plate 220 may comprise a compression screw mating feature 221, and an extended geometry 222.

In FIG. 22B, a top, perspective view of an alternative embodiment of a dorsal bone plate with mating feature for compression screw fixated to a corrected metatarsal generally represented by reference numeral 220 and utilized in a ROSE bunionectomy procedure is shown, according to the present disclosure. The dorsal plate 220 may comprise a compression screw mating feature 221, an extended geometry 222, a compression screw 223, locking or non-locking screws 224, a dorsal metatarsal fragment 225, and a plantar metatarsal fragment 226.

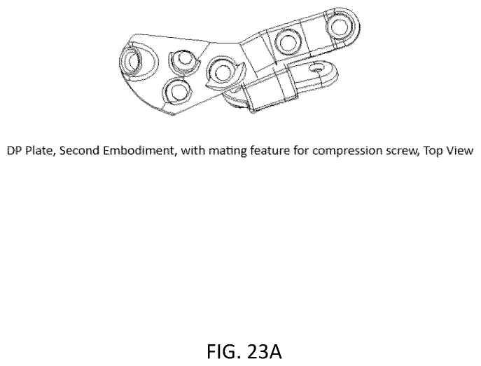

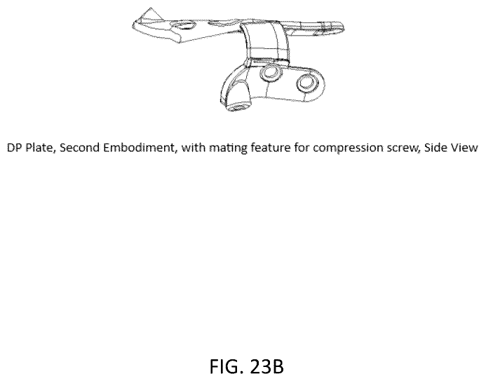

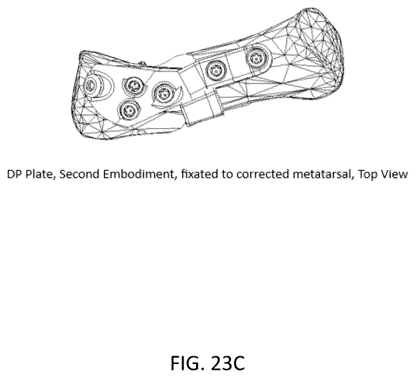

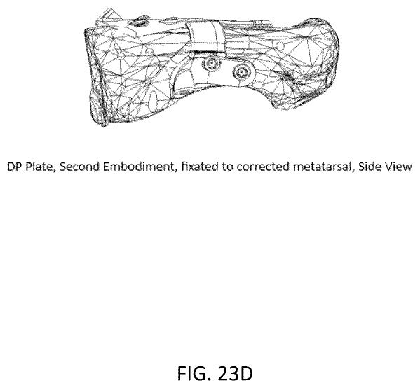

In FIG. 23A, a top, perspective view of an alternative embodiment of a dorsal plantar (DP) plate with mating feature for compression screw generally represented by reference numeral 230 and utilized in a ROSE bunionectomy procedure is shown, according to the present disclosure. The dorsal plantar (DP) plate 230 may comprise a compression screw mating feature 231, and an extended geometry 232.

In FIG. 23B, a side, perspective view of an alternative embodiment of a dorsal plantar (DP) plate with mating feature for compression screw generally represented by reference numeral 230 and utilized in a ROSE bunionectomy procedure is shown, according to the present disclosure. The dorsal plantar (DP) plate 230 may comprise a compression screw mating feature 231, and an extended geometry 232.

In FIG. 23C, a top, perspective view of an alternative embodiment of a dorsal plantar (DP) plate with mating feature for compression screw fixated to a corrected metatarsal generally represented by reference numeral 230 and utilized in a ROSE bunionectomy procedure is shown, according to the present disclosure. The dorsal plantar (DP) plate 230 may comprise a compression screw mating feature 231, an extended geometry 232, a compression screw 233, locking or non-locking screws 234, a dorsal metatarsal fragment 235, and a plantar metatarsal fragment 236.

In FIG. 23D, a side, perspective view of an alternative embodiment of a dorsal plantar (DP) plate with mating feature for compression screw fixated to a corrected metatarsal generally represented by reference numeral 230 and utilized in a ROSE bunionectomy procedure is shown, according to the present disclosure. The dorsal plantar (DP) plate 230 may comprise a compression screw mating feature 231, an extended geometry 232, a compression screw 233, locking or non-locking screws 234, a dorsal metatarsal fragment 235, and a plantar metatarsal fragment 236.

### Dorsal Plantar (DP) Bone Plate Screw (i.e., Clamping Screw)

The novel dorsal plantar (DP) bone plate screw, DP screw, also known as the clamping screw, is an integral part of the ROSE osteotomy and fixation procedure. The cannulated style bone screw may comprise a non-locking head with torx, or similar, drive. The shank of the clamping screw may be non-threaded, while the lower portion of the screw may comprise an external thread. The major diameter of the external thread may be smaller than the diameter of the non-threaded shank portion.

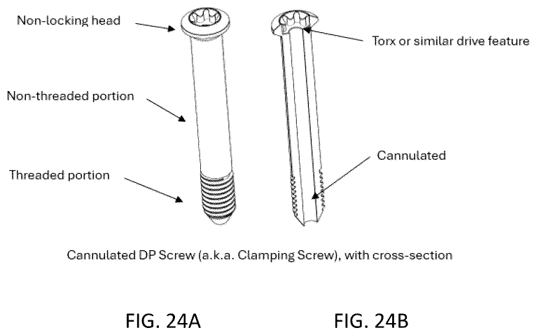

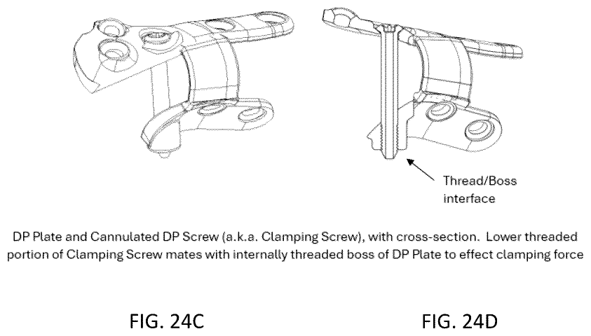

In FIGS. 24A and 24B, a perspective and cross-sectional views of a cannulated dorsal plantar (DP) screw (i.e., clamping screw) generally represented by reference numeral 240 and utilized in a ROSE bunionectomy procedure are shown, according to the present disclosure. The clamping screw 240 may comprise a threaded portion 241, a non-threaded portion 242, a non-locking head 243, a cannulated bottom 244, and a torx or similar drive feature top 245.

The clamping screw is used with the novel DP bone plate (DP plate). The clamping screw passes through a screw clearance hole on the dorsal portion of the DP plate, and continues through the dorsal metatarsal fragment, osteotomy cut, and plantar metatarsal fragment. A K-wire, or similar, can be used to guide the clamping screw through this plate clearance hole and the metatarsal fragments for proper alignment with the internally threaded boss of the DP plate plantar portion. As the external threads of the clamping screw engage the internal threads of the DP plate, the clamping screw interacts with the DP plate, at both the dorsal and plantar portions, to create a clamping effect on the corrected metatarsal.

In FIGS. 24C and 24D, a perspective and cross-sectional views of a dorsal plantar (DP) bone plate (i.e., DP plate) and cannulated dorsal plantar (DP) screw (i.e., clamping screw) having a lower threaded portion of the clamping screw mate with an internally threaded boss of the DP plate to effect a clamping force generally represented by reference numeral 240 and utilized in a ROSE bunionectomy procedure are shown, according to the present disclosure. The clamping screw 240 may comprise a threaded portion 241, a non-threaded portion 242, a non-locking head 243, a cannulated bottom 244, a torx or similar drive feature top 245, a thread/boss interface 246, and a dorsal plantar (DP) bone plate 247.

### Specific Use Case Example

### Example of a Bunionectomy Utilizing the ROSE-Reverdin Osteotomy

The unique and advanced system and method for a bunionectomy described herein corrects a large gap of space between the metatarsal bones.

A unique surgical procedure corrects advanced bunions without the need for joint fusion, setting it apart from other methods like Lapidus and/or Lapiplasty®. Joint fusion involves fusing bones together to correct the bunion.

This can lead to longer recovery times and might limit the patient's foot movement. By utilizing this approach, joint fusion is avoided, aiming to bring the patient back to his/her regular activities, reduce pain, and make his/her feet feel more comfortable.

Avoiding joint fusion means quicker healing and less discomfort. The patient can even put some weight on his/her foot immediately after the surgery, making the recovery easier.

The steps of the advanced bunion surgical procedure are as follows:
Step 1: Bunion Bump Removal—Start by carefully removing the bunion bump, making the bone smooth and ready for correction.
Step 2: Precise Bone Cut—A precise bone cut is made along the midshaft to the base of the first metatarsal joint, preserving the joint and promoting quicker healing.
Step 3: Axis Point Pin Placement—To correct the deformity, a pin is placed at the axis point, enabling the physician to rotate the bone, effectively closing the large gap and correcting the bunion.
Step 4: Bone Rotation—Gentle rotation of the bone from the axis point ensures precise correction without bone shortening or elevation.
Step 5: Screw and Bone Plate Application—With the rotation complete, a screw is used to provide stability, and a bone plate is applied over the cut, allowing for early weight-bearing.
Step 6: Optional Cartilage Realignment—When necessary, an additional wedge cut is made to realign the cartilage and joint, allowing for optimal joint function.
Step 7: Closing the Wedge—If a wedge cut was made, a second screw is placed to close the wedge securely. The advanced bunion surgical procedure disclosed herein offers numerous advantages over traditional Lapidus and/or Lapiplasty® techniques.

Unlike those methods that require joint fusion, the unique approach shown above and described herein preserves joint function, reducing the risk of complications and promoting a quicker and more comfortable recovery.

### ALTERNATIVES, EQUIVALENTS AND RANGES

The broadest operable range is an entire health care ecosystem—e.g., any person with severe deformities having a high intermetatarsal angle and requiring closure via osteotomy (bone cut) of the first and/or fifth metatarsal in the midshaft or proximal aspect of the bone. For example, the ROSE osteotomy achieves correction of the intermetatarsal angle via rotation of the first and/or fifth metatarsal about a proximal based axis point. Further, for example, the Reverdin osteotomy, is required to correct the deviated articular cartilage deformity at the head of the first metatarsal and/or additional slight correction of the IM angle. Correction with the Reverdin osteotomy, is achieved by wedging of the cut to realign the cartilage and translation of the head of the first metatarsal for further IM correction, wherein a unique jig described herein is used to achieve this cut and fixation.

The techniques described herein are exemplary and should not be construed as implying any particular limitation on the present disclosure. It should be understood that various alternatives, combinations and modifications could be devised by those skilled in the art. For example, steps associated with the processes described herein can be performed in any order, unless otherwise specified or dictated by the steps themselves. The present disclosure is intended to embrace all such alternatives, modifications and variances that fall within the scope of the appended claims.

The terms “proximal” and “distal” are to be interpreted as specifying the presence of stated features as being used in structures that are considered to have a beginning and an end (such as the upper limb, lower limb and blood vessels). They describe the position of a structure with reference to its origin-proximal means closer to its origin, distal means further away.

The terms “superior” and “inferior” are to be interpreted as specifying the presence of stated features as superior (from Latin super ‘above’) describing what is above something and inferior (from Latin inferus ‘below’) describes what is below it. For example, in the anatomical position, the most superior part of the human body is the head and the most inferior is the feet.

Certain directional anatomical terms appear throughout this disclosure. These terms are essential for describing the relative locations of different body structures and positions. For instance, an anatomist might describe one band of tissue as “inferior to” another or a physician might describe a tumor as “superficial to” a deeper body structure. To avoid confusion, this disclosure is describing the locations of particular body parts.

Anterior (or ventral) Describes the front or direction toward the front of the body. For example, the stomach is anterior to the spine.

Posterior (or dorsal) Describes the back or direction toward the back of the body. For example, the popliteus is posterior to the patella.

Superior (or cranial) describes a position above or higher than another part of the body proper. For example, the orbits are superior to the oris.

Inferior (or caudal) describes a position below or lower than another part of the body proper; near or toward the tail (e.g., in humans, the coccyx, or lowest part of the spinal column). For example, the pelvis is inferior to the abdomen.

Lateral describes the side or direction toward the outside of the body. For example, the fifth metatarsal is lateral to the first metatarsal.

Medial describes the middle or direction toward the midline of the body. For example, the hallux is the most medial toe.

Proximal describes a position in a limb that is nearer to the point of attachment or the trunk of the body. For example, the knee is proximal to the ankle.

Distal describes a position in a limb that is farther from the point of attachment or the trunk of the body. For example, the ankle is distal to the knee.

Superficial describes a position closer to the surface of the body. For example, the skin is superficial to the bones.

Deep describes a position farther from the surface of the body. For example, the brain is deep to the skull.

The terms “comprises” or “comprising” are to be interpreted as specifying the presence of the stated features, integers, steps or components, but not precluding the presence of one or more other features, integers, steps or components or groups thereof.

What is claimed is:

1. A system for utilizing a bone cut to achieve correction of deformities having an intermetatarsal angle, the system comprising:
a first bone cut jig;
at least one support that secures the first bone cut jig to a metatarsal;
a first jig cut guide groove in the first bone cut jig, wherein a cutting tool traverses the first jig cut guide groove, thereby creating a bone cut through the metatarsal from medial to lateral, wherein the bone cut is oriented from dorsal distal to plantar proximal;
a plurality of dorsal holes disposed in a dorsal surface of the first bone cut jig, thereby utilizing said support to hold the first bone cut jig to a dorsal surface of the metatarsal, wherein at least one of the plurality of dorsal holes is proximal and at least one of the plurality of dorsal holes is distal to the bone cut, dorsal exit point;
a plurality of medial holes disposed in a medial surface of the first bone cut jig, thereby utilizing said support to hold the first bone cut jig to a medial surface of the metatarsal, wherein at least one of the plurality of medial holes is superior proximal and at least one of the plurality of medial holes is distal inferior to the first jig cut guide groove;
a bone cut fixation plate placed over the dorsal surface of the metatarsal, wherein the bone cut fixation plate has a slight bend distally; and
a plurality of fixation fasteners that secure the bone cut fixation plate to the dorsal surface of the metatarsal, wherein said support is utilized, thereby securing the bone cut fixation plate proximal and distal as the plurality of fixation fasteners are being inserted.

2. The system of claim 1, further comprising:
a bone cut IM correction clamp placed on the dorsal surface of the metatarsal, thereby as the bone cut IM correction clamp is closed, the metatarsal is rotated laterally closing or reducing the intermetatarsal (IM) angle, thus correcting the deformities.

3. The system of claim 2, further comprising:
a second bone cut jig;
a plurality of fixation holes disposed in a dorsal surface and a medial surface of the second bone cut jig to stabilize the second bone cut jig on a head of the metatarsal;
a plurality of supports for utilizing with the plurality of fixation holes, thereby stabilizing the second bone cut jig to the head of the metatarsal; and
at least one second jig cut guide groove.

4. The system of claim 3, wherein the at least one second jig cut guide groove is selected from the group consisting of: a wedge shaped and dorsal, a straight linear shaped and medial, and a fully through a dorsal surface.

5. The system of claim 1, wherein the first bone cut jig is a ROSE bone cut jig.

6. The system of claim 3, wherein the second bone cut jig is a Reverdin bone cut jig.

7. The system of claim 1, wherein the metatarsal is a first metatarsal.

8. The system of claim 1, wherein the metatarsal is a fifth metatarsal.

9. The system of claim 3, wherein the metatarsal is a first metatarsal.

10. A method for utilizing a bone cut to achieve correction of deformities having an intermetatarsal angle, the method comprising:
fitting a first bone cut jig;
inserting at least one support that secures the first bone cut jig to a metatarsal;
fitting a first jig cut guide groove in the first bone cut jig, wherein a cutting tool traverses the first jig cut guide groove, thereby creating a bone cut through the metatarsal from medial to lateral, wherein the bone cut is oriented from dorsal distal to plantar proximal;
utilizing a plurality of dorsal holes disposed in a dorsal surface of the first bone cut jig, thereby utilizing said support to hold the first bone cut jig to a dorsal surface of the metatarsal, wherein at least one of the plurality of dorsal holes is proximal and at least one of the plurality of dorsal holes is distal to the bone cut, dorsal exit point;
utilizing a plurality of medial holes disposed in a medial surface of the first bone cut jig, thereby utilizing said support to hold the first bone cut jig to a medial surface of the metatarsal, wherein at least one of the plurality of medial holes is superior proximal and at least one of the plurality of medial holes is distal inferior to the first jig cut guide groove;
fitting a bone cut fixation plate placed over the dorsal surface of the metatarsal, wherein the bone cut fixation plate has a slight bend distally; and
utilizing a plurality of fixation fasteners that secure the bone cut fixation plate to the dorsal surface of the metatarsal, wherein said support is utilized, thereby securing the bone cut fixation plate proximal and distal as the plurality of fixation fasteners are being inserted.

11. The method of claim 10, further comprising:
utilizing a bone cut IM correction clamp placed on the dorsal surface of the metatarsal, thereby as the bone cut IM correction clamp is closed, the metatarsal is rotated laterally closing or reducing the intermetatarsal (IM) angle, thus correcting the deformities.

12. The method of claim 11, further comprising:
fitting a second bone cut jig;
utilizing a plurality of fixation holes disposed in a dorsal surface and a medial surface of the second bone cut jig to stabilize the second bone cut jig on a head of the metatarsal;
using a plurality of supports for utilizing with the plurality of fixation holes, thereby stabilizing the second bone cut jig to the head of the metatarsal; and
utilizing at least one second jig cut guide groove.

13. The method of claim 12, wherein the at least one second jig cut guide groove is selected from the group consisting of: a wedge shaped and dorsal, a straight linear shaped and medial, and a fully through a dorsal surface.

14. The method of claim 10, wherein the first bone cut jig is a ROSE bone cut jig.

15. The method of claim 12, wherein the second bone cut jig is a Reverdin bone cut jig.

16. The method of claim 10, wherein the metatarsal is a first metatarsal.

17. The method of claim 10, wherein the metatarsal is a fifth metatarsal.

18. The method of claim 12, wherein the metatarsal is a first metatarsal.

19. A system for utilizing a bone cut to achieve correction of deformities of a head of a metatarsal, the system comprising:
a bone cut jig;
a plurality of fixation holes disposed in a dorsal surface and a medial surface of the bone cut jig to stabilize the bone cut jig on the head of the metatarsal;
a plurality of supports for utilizing with the plurality of fixation holes, thereby stabilizing the bone cut jig to the head of the metatarsal; and
at least one jig cut guide groove.

20. The system of claim 19, wherein the at least one jig cut guide groove is selected from the group consisting of: a wedge shaped and dorsal, a straight linear shaped and medial, and a fully through a dorsal surface.

21. The system of claim 19, wherein the bone cut jig is a Reverdin bone cut jig.

22. The system of claim 19, wherein the metatarsal is a first metatarsal.

23. A method for utilizing a bone cut to achieve correction of deformities of a head of a metatarsal, the method comprising:
fitting a bone cut jig;
utilizing a plurality of fixation holes disposed in a dorsal surface and a medial surface of the bone cut jig to stabilize the bone cut jig on the head of the metatarsal;
using a plurality of supports for utilizing with the plurality of fixation holes, thereby stabilizing the bone cut jig to the head of the metatarsal; and
utilizing at least one jig cut guide groove.

24. The method of claim 23, wherein the at least one jig cut guide groove is selected from the group consisting of: a wedge shaped and dorsal, a straight linear shaped and medial, and a fully through a dorsal surface.

25. The method of claim 23, wherein the bone cut jig is a Reverdin bone cut jig.

26. The method of claim 23, wherein the metatarsal is a first metatarsal.
